(19) United States
(12) Patent Application Publication
(10) Pub. No.: US 2024/0173127 A1
(43) Pub. Date: May 30, 2024

(54) PROSTHETIC HEART VALVE DEVICE, FRAME, DELIVERY SYSTEM, INTERVENTIONAL SYSTEM AND RELATED METHODS

(71) Applicant: LAGUNA TECH USA, INC., Irvine, CA (US)

(72) Inventors: CHARLIE CLAPP, Irvine, CA (US); GILBERT MADRID, Irvine, CA (US)

(21) Appl. No.: 18/431,953

(22) Filed: Feb. 3, 2024

Related U.S. Application Data
(63) Continuation of application No. PCT/IB2022/057187, filed on Aug. 3, 2022.
Application No. PCT/IB2022/057187 is a continuation-in-part of application No. 17/394,190, filed on Aug. 4, 2021, now Pat. No. 11,806,233.
(60) Provisional application No. 63/254,994, filed on Oct. 12, 2021.
(60) Provisional application No. 63/311,577, filed on Feb. 18, 2022.
(60) Provisional application No. 63/394,299, filed on Aug. 2, 2022.

(51) Int. Cl. A61F 2/24

(52) U.S. Cl. CPC A61F 2/2418; A61F 2210/0014; A61F 2210/0057; A61F 2220/0033; A61F 2250/001

(57) ABSTRACT

The present invention provides a prosthetic heart valve device that has a frame, and a leaflet assembly having a plurality of leaflets that are secured to the frame. The frame is defined by an annular body and has three spaced-apart commissure regions, each commissure region having a commissure post extending from a proximal outflow end of the frame. A first clipping arm and a second clipping arm extend from opposite sides of each commissure post, each clipping arm extending from each commissure post at an obtuse angle with respect to each commissure post. Each clipping arm has a free end with a tip provided at the free end. The body has a first diameter at a location where the tips of the clipping arms are located, and the tips of the clipping arms extend away from define a second diameter, with the second diameter being greater than the first diameter.

### INCORPORATION BY REFERENCE TO ANY PRIORITY APPLICATIONS

This application is a continuation of international application no. PCT/IB2022/057187, filed on Aug. 3, 2022, which claims the benefit of priority to U.S. patent application Ser. No. 17/394,190, filed on Aug. 4, 2021, now U.S. Pat. No. 11,806,233, U.S. Provisional Application No. 63/254,994, filed on Oct. 12, 2021, U.S. Provisional Application No. 63/311,577, filed on Feb. 18, 2022, and U.S. Provisional Application No. 63/394,299, filed on Aug. 2, 2022, all of which are incorporated by reference in their entirety herein for all purposes. Priority is claimed pursuant to 35 U.S.C. § 120 and 35 U.S.C. § 119.

### TECHNICAL FIELD

The present invention relates to a prosthetic heart valve device, and in particular, to a prosthetic heart valve for use in treating aortic valve insufficiency.

### BACKGROUND

Aortic valve insufficiency (AI), also known as aortic regurgitation (AR), is a serious and potentially fatal structural heart disease afflicting millions of patients worldwide. AI is characterized by volume overload and eccentric hypertrophy associated with left ventricular (LV) cavity structural modifications and progressive dysfunction. This results in the dilatation of the aortic fixed end/annulus, which leads to aortic regurgitation. Left untreated, this disease can become progressively worse and may eventually lead to patient death.

To-date, there are only two known minimally invasive transcatheter aortic valve implantation devices for treating AI disease. The first device is manufactured by Jena Valve, and utilizes a frame design with “feeler” arches, to align with the native anatomy, and to clip the native valve leaflets during deployment. However, the JenaValve design is difficult to deliver and deploy due to its open cell design, and does not have any structure to prevent native leaflet interaction with the prosthetic leaflets. The JenaValve device also has a significant asymmetric construction that includes different cell sizes, and a notched design for leaflet attachment. All these features make the device crimping very challenging, and thus the deployment can be difficult to control.

The second device is manufactured by JC Medical, and utilizes a two-piece design, which has U-shaped anchor rings that are deployed in the native cusps, followed by the self-expanding valve endoprosthesis. The two separate pieces are anchored together utilizing suture/wire, which allow for the potential to fail during or after implantation, potentially causing migration and/or device embolization. The major disadvantage of this device is a metal-on-metal design that can increase the profile and affect the long-term durability of the valve.

The transcatheter aortic valve implantation devices for treating AI disease are to be contrasted with traditional transcatheter valve designs, which are indicated for the treatment of aortic stenosis. The traditional stenotic valve provides a secure ring to deploy and anchor a native valve. However, in a pure AI disease state, there is not a secure ring to anchor inside. Therefore, utilizing the native anatomy to anchor the valve is more difficult in non-stenotic valves that are used to treat AI.

Thus, there remains a need for a prosthetic heart valve that can be used to treat AI, and which overcomes the deficiencies of the existing devices.

### SUMMARY

The present invention provides a prosthetic heart valve that can be used effectively to treat AI while avoiding the drawbacks experienced by the known devices.

In order to accomplish the objects of the present invention, the present invention provides a prosthetic heart valve device that has a frame, and a leaflet assembly having a plurality of leaflets that are secured to the frame.

The frame is defined by an annular body that is defined by an arrangement of cells. The frame has three spaced-apart commissure regions, each commissure region having a commissure post extending from a proximal outflow end of the frame. A first clipping arm and a second clipping arm extend from opposite sides of each commissure post, each clipping arm extending from each commissure post at an angle that ranges from 90 to 180 degrees with respect to each commissure post. Each clipping arm has a free end with a tip provided at the free end. The body has a first diameter at a location where the tips of the clipping arms are located, and the tips of the clipping arms extend away from define a second diameter, with the second diameter being greater than the first diameter.

The present invention also provides a method of securing the prosthetic heart valve device at an aortic annulus that includes a plurality of native leaflets. This method includes the steps of crimping the heart valve device inside a delivery system, delivering the heart valve device to the annulus, and deploying the heart valve device at the annulus with at least some of the native leaflets positioned between the clipping arms and the body.

According to another embodiment, some of the native leaflets can also be positioned around an external surface of some of the clipping arms.

The method of the present invention can also include the steps of:
advancing the delivery system through the aortic arch and the ascending aorta of the patient with a distal portion of the delivery system passing through the aortic annulus into the ventricle;
retracting a portion of the delivery system so that the clipping arms are exposed in the ventricle;
retracting the delivery system and the heart valve device so that the clipping arms have completely cleared the aortic annulus and are now positioned inside the aortic fixed end;
with distal ends of the clipping arms positioned above the native aortic valve, advancing the heart valve device distally until the clipping arms drop into the cusps of the native leaflets; and
retracting the remainder of the delivery system to deploy the body of the frame at the aortic annulus.

The present invention provides a prosthetic heart valve device, and a method of deployment thereof, that can be effectively deployed at an aortic annulus in a manner which minimizes post-deployment shifting or movement of the deployed heart valve devices.

The present application provides a frame for a prosthetic heart valve device, including: an inner frame having a meshed cylindrical structure, the inner frame having relative compressed and expanded configurations depending on the radial deformation, and a support device for driving the inner frame to switch to the expanded configuration is allowed to be placed inside the inner frame; and
a plurality of groups of clipping arms located at an outer periphery of the inner frame and spaced apart from each other in the circumference of the frame, each clipping arm having opposite fixed end and free end, the fixed end being directly or indirectly connected with the inner frame, and the fixed ends of the clipping arms in the same group are adjacent to each other, and wherein the clipping arm is made of memory material and has configurations of
a loaded configuration, in which the inner frame assumes the compressed configuration, and the clipping arms contact the inner frame; and
a released configuration, in which the inner frame assumes the expanded configuration, the free end of each clipping arm expands radially outward, with a space defined between the free end of each clipping arm and the inner frame to allow entry of the native leaflet, wherein the free ends of at least two clipping arms in the same group tend to extend away from each other, and the free ends of at least two clipping arms in adjacent groups tend to extend close to each other.

The present application provides a frame for a prosthetic heart valve device, including:
an inner frame having a meshed cylindrical structure, which has relative compressed configuration and expanded configuration depending on the radial deformation, and a support device for driving the inner frame to transform into the expanded configuration can be placed within the inner frame;
a connecting ring fixed with the outflow end of the inner frame and provided with a plurality of connecting regions at intervals; and
a plurality of groups of clipping arms which are located at an outer periphery of the inner frame and spaced in the circumferential direction of the frame, each clipping arm having opposite fixed end and free end, and the fixed ends of clipping arms in the same group are located at the same connecting region.

The clipping arm is made of memory material and has configurations of
a loaded configuration, in which the inner frame assumes the compressed configuration, and the clipping arms contact the inner frame; and
a released configuration, in which the inner frame assumes the expanded configuration, the free end of each clipping arm expands radially outward, with a space defined between the free end of each clipping arm and the inner frame to allow entry of the native leaflet.

The present application provides a frame for a prosthetic heart valve device, including:
an inner frame having a meshed cylindrical structure, which has relative compressed configuration and expanded configuration depending on the radial deformation, and a support device for driving the inner frame to transform into the expanded configuration can be placed within the inner frame; and
clipping arms, each of which has opposite fixed end and free end, wherein the fixed end is connected with the inner frame, and the clipping arm satisfies at least one of the following conditions with respect to the axis of the inner frame:
the circumferential distribution region M1 of the fixed end has a central angle greater than 15 degrees with respect to the axis; and
the length of the axial distribution region M3 of the clipping arm relative to the axis is greater than 5 mm;

The clipping arm is made of memory material and has configurations of:
a loaded configuration, in which the inner frame assumes the compressed configuration, and the clipping arms contact the inner frame; and
a released configuration, in which the inner frame assumes the expanded configuration, the free end of each clipping arm expands radially outward, with a space defined between the free end of each clipping arm and the inner frame to allow entry of the native leaflet.

The present application provides a prosthetic heart valve device including a frame for a prosthetic heart valve device according to the present application, and valve leaflets connected to the frame and located in a blood flow channel. The valve leaflets cooperate with each other for controlling the opening or closing of the blood flow channel.

The present application provides a delivery system for a prosthetic heart valve device including:
a support device that is switchable between the inflated and deflated configurations under fluid; and
an outer sheath that is slidably engaged with the periphery of the support device, the radial gap between the outer sheath and the support device being a loading zone.

The prosthetic heart valve device of the present application is placed in the loading zone in a compressed configuration.

The present application discloses a positioning method for the prosthetic heart valve device for positioning any of the prosthetic heart valve devices according the present invention, and the positioning method includes:
delivering the prosthetic heart valve device to a predetermined site, in which the inner frame is in a compressed configuration, the clipping arms are in a loaded configuration, and the support device is in a deflated configuration;
driving the outer sheath to release the free ends of the clipping arms, thereby expanding the free ends of the clipping arms;
adjusting the position of the inner frame such that the free end of the at least one clipping arm is located outside the native leaflet; and
driving the support device to the inflated configuration and releasing the inner frame and the fixed ends of the clipping arms, so that the inner frame transforms into the expanded configuration and the clipping arms transform into the released configuration.

The present application discloses a prosthetic aortic valve device, having an inflow end and an outflow end opposite to each other, the prosthetic aortic valve device including:
an inner frame having a meshed cylindrical structure, which is radially deformable and has relative compressed configuration and expanded configuration, wherein the interior of the inner frame is configured as an axially through blood flow channel;
leaflets connected to the inner frame and cooperated with each other to control the opening and closing of the blood flow channel; and
guiding members arranged in sequence in the circumferential direction of the inner frame, and the position thereof respectively aligned with the leaflets in the circumferential direction, wherein each guiding member includes a root connected with the inner frame and a wing extending from the root further toward the inflow end, the guiding member is made of a memory material and is configured to be switchable in the following configurations:
in the loaded configuration, the portions of the guiding member are radially closed to the inner frame in the compressed configuration;
in a transition configuration, the roots of the guiding members remain gathered to adapt the compressed configuration of the inner frame, the wings self-stretches outside of the inner frame, with an accommodation space formed between the outer wall of the inner frame and the wings for receiving the native leaflets; and
in a released configuration, the roots of the guiding members move far away from each other to adapt the expanded configuration of the inner frame.

The present application discloses a delivery system for a prosthetic aortic valve device for loading and delivering the prosthetic aortic valve device described herein, the delivery system having opposite distal and proximal ends and including:
a balloon device switchable between an inflated configuration and a deflated state under the action of a fluid;
an outer sheath which is slidably fitted on the outer periphery of the balloon device, and a radial gap between the outer sheath and the balloon device is a loading zone for receiving the prosthetic aortic valve device; and
a control handle, wherein both the proximal ends of the balloon device and the outer sheath extend to the control handle with the outer sheath slidably fit with the control handle.

The present application discloses an interventional system including a prosthetic aortic valve device as described herein, and a delivery system for the prosthetic aortic valve device as described herein.

The present application discloses a method for using an interventional system, including:
delivering the prosthetic aortic valve device to a predetermined site, during which, the inner frame is in a compressed configuration, the guiding members are in a loaded configuration, and the balloon device is in a deflated configuration;
proximally retracting the outer sheath to expose the wings of the guiding members to drive the guiding members into a transition configuration;
obtaining the position of the guiding members relative to the valvular sinuses, rotating the support device and driving the inner frame to move synchronously when the guiding members are misaligned, so that the wings of the guiding members are aligned with and enter the valvular sinuses; and
driving the balloon device to the inflated configuration to release the inner frame and the roots of the guiding members so that the inner frame transforms into the expanded configuration and the guiding members transform into the released configuration.

The present application discloses a prosthetic aortic valve device having opposite inflow and outflow ends, the prosthetic aortic valve device including:
an inner frame having a meshed cylindrical structure, which is radially deformable and has relative compressed configuration and expanded configuration, wherein the interior of the inner frame is configured as an axially through blood flow channel;
valve leaflets connected to the inner frame and cooperating with each other to control the blood flow channel, with the two adjacent valve leaflets joined at the commissure region of the inner frame; and
positioning members arranged in sequence in the circumferential direction of the inner frame, one end of each of which is connected to the inner frame and the other end extends toward the inflow end, wherein a spacing region is formed at the outer peripheral region of the inner frame between two adjacent commissure regions, and the positioning members avoid the spacing region.

### BRIEF DESCRIPTION OF THE DRAWINGS

FIG. 1 is a side perspective view of the frame of a prosthetic heart valve device according to a first embodiment of the present invention.

FIG. 2 is a flattened view of a portion of the frame of FIG. 1.

FIG. 3 is a flattened view of the entire frame of FIG. 1.

FIG. 4 is a top perspective view of the frame of FIG. 1.

FIG. 5 is a side perspective view of the prosthetic heart valve device according to the first embodiment of the present invention.

FIG. 6 is a top perspective view of the device of FIG. 5.

FIG. 7 is a top view of the device of FIG. 5.

FIG. 8 is a side perspective view showing the device of FIG. 5 clipping a native valve leaflet.

FIG. 9 is a bottom perspective view showing the device of FIG. 5 clipping a native valve leaflet.

FIG. 10A illustrates the device of FIG. 5 retained in a compressed configuration inside a delivery system.

FIG. 10B illustrates the various components of the delivery system of FIG. 10A shown in the fully deployed configuration, without the device of FIG. 5.

FIG. 10C illustrates the various components of the delivery system of FIG. 10A shown in the fully deployed configuration, with the device of FIG. 5 compressed on the inner tube.

FIGS. 11A-11G illustrate how the device of FIG. 5 is delivered to an aortic annulus of a patient's heart and deployed at the aortic annulus.

FIG. 12 is a side perspective view of the frame of a prosthetic heart valve device according to a second embodiment of the present invention.

FIG. 13 is a flattened view of a portion of the frame of FIG. 12.

FIG. 14 is a side perspective view of the frame of a prosthetic heart valve device according to a third embodiment of the present invention.

FIG. 15 is a side perspective view of the frame of a prosthetic heart valve device according to a fourth embodiment of the present invention.

FIG. 16 is a side perspective view of the frame of a prosthetic heart valve device according to a fifth embodiment of the present invention.

FIG. 17A illustrates a modification that can be made to the frame of FIG. 1.

FIG. 17B illustrates the frame of FIG. 17A secured at a native annulus.

FIG. 18 A illustrates another modification that can be made to the frame of FIG. 1.

FIG. 18B illustrates the frame of FIG. 18A secured at a native annulus.

FIG. 19 is a perspective view of a frame for a prosthetic heart valve device according to an embodiment;

FIG. 20a to FIG. 20d illustrate different examples of the clipping arms in front view, respectively;

FIG. 20e illustrates an asymmetric configuration of the clipping arms;

FIG. 21a illustrates the engagement between the clipping arm and the inner frame in a side view;

FIG. 21b illustrates the engagement between the clipping arm and the native leaflet;

FIG. 22a to FIG. 22d illustrate different examples of the clipping arms in a side view, respectively;

FIG. 23a and FIG. 23b illustrate the engagement between the clipping arms and the inner frame according to different embodiments in a top view, respectively;

FIG. 24a illustrates the engagement between the fixed end of the clipping arm and the commissure region;

FIG. 24b to FIG. 24d illustrate the assemble of the fixed end and the commissure region in different embodiments, respectively;

FIG. 25 is a perspective view of a frame of a prosthetic heart valve device according to another embodiment;

FIGS. 26 to 29 illustrate the engagement of the fixed end of the clipping arm and the commissure post, respectively;

FIG. 30a to FIG. 30d illustrate the engagement between the frame in FIG. 25 with different clipping arms, respectively;

FIG. 31 is a perspective view of the clipping arm inside the inner frame;

FIG. 32 is a flattened view of a frame of a prosthetic heart valve device according to an embodiment;

FIG. 33a is a view of a connecting ring according to an embodiment;

FIG. 33b is a view showing the engagement between the connecting ring in FIG. 33a and the frame;

FIG. 33c is a view of a connecting ring according to another embodiment;

FIG. 33d is a view showing the engagement between the connecting ring in FIG. 33c and the frame;

FIG. 33e is a view of a connecting ring according to a further embodiment;

FIG. 33f is a view showing the engagement between the connecting ring in FIG. 33e and the frame;

FIGS. 34a and 34b are views of a connecting ring and a clipping arm formed in one piece according to different embodiments, respectively;

FIG. 35 is a perspective view of a frame of a prosthetic heart valve device according to another embodiment;

FIGS. 36a and 36b are views showing the connecting ring and the inner frame being connected by a flexible member, respectively;

FIGS. 36c and 36d are other views of FIGS. 36a and 36b, respectively;

FIG. 37a is a perspective view of a frame with reinforced clipping arms according to an embodiment;

FIG. 37b is a flattened view of a frame with reinforced clipping arms according to an embodiment;

FIG. 37c is a top view of the frame of FIG. 37b;

FIG. 37d is a flattened view of a frame with reinforced clipping arms according to another embodiment;

FIG. 37e is a view of clipping arms configured as mesh bands;

FIG. 37f is a view of clipping arms with thickened edges;

FIG. 37g is a view of clipping arms with positioning structures at the free ends thereof;

FIG. 37h is a view of clipping arms with various positioning structures provided thereon;

FIG. 37i is a view of clipping arms with sleeves;

FIG. 37j is a view of clipping arm with sleeves and positioning structures simultaneously provided thereon;

FIG. 37k is a view of reinforced clipping arms formed in one piece;

FIG. 37l is a view of reinforced clipping arms formed in one piece with different extensions;

FIG. 37m is a view of asymmetric reinforced clipping arms formed in one piece with different extensions;

FIG. 38a is a side view of a frame with a reinforced clipping arm according to another embodiment;

FIG. 38b is a view showing the engagement between the frame in FIG. 38a and native leaflet;

FIG. 38c is a perspective view of a prosthetic heart valve device with reinforced clipping arms according to one embodiment;

FIG. 38d is a view showing the engagement between the prosthetic heart valve device of FIG. 38c and the native valve leaflet;

FIGS. 38e and 38f are views showing the engagement between the fixed ends of the clipping arms, respectively;

FIG. 38g is a view of reinforced clipping arms according to another embodiment;

FIG. 38h shows the engagement between the clipping arms and the inner frame in FIG. 38g;

FIG. 38i is a view comparing the loaded configuration and the released configuration of the clipping arms of FIG. 38g;

FIG. 38j illustrates different extensions of the reinforced clipping arms;

FIG. 38k illustrates an asymmetrical configuration of the reinforced clipping arms;

FIG. 39a is a perspective view of a prosthetic heart valve device according to another embodiment;

FIG. 39b illustrates the engagement between the prosthetic heart valve device of FIG. 39a and the native valve leaflet;

FIG. 40 illustrates a slot in an embodiment;

FIG. 41a to FIG. 41c are views illustrating different operations of the delivery system according to an embodiment;

FIGS. 42a to 42g are views illustrating the operations of the delivery system according to an embodiment;

FIG. 43 a to FIG. 43 d are views showing the pre-expansion of the delivery system;

FIG. 44 is a view showing the location of the aorta and coronary arteries in the heart;

FIG. 45 illustrates the circumferential engagement between the prosthetic aortic valve device and the aortic valve;

FIG. 46a is a perspective view of a prosthetic aortic valve device according to an embodiment;

FIG. 46b is a perspective view of the prosthetic aortic valve device of FIG. 46a in another view;

FIG. 47a is a front view of the inner frame of the prosthetic aortic valve device of FIG. 46b in the released configuration in the form of a straight cylinder (with leaflets not shown).

FIG. 47b is a left side view of the prosthetic aortic valve device of FIG. 47a;

FIG. 48 is a front view of the prosthetic aortic valve device in a loaded configuration in one embodiment;

FIG. 49 is a front view of the prosthetic aortic valve device in a transition configuration in one embodiment;

FIG. 50 is a perspective view of the prosthetic aortic valve device of FIG. 48;

FIG. 51 is a perspective view of the prosthetic aortic valve device of FIG. 49;

FIG. 52a is a front view of the prosthetic aortic valve device in a released configuration in one embodiment;

FIG. 52b is a left side view of the prosthetic aortic valve device of FIG. 52a;

FIG. 52c is a perspective view of the prosthetic aortic valve device of FIG. 52a;

FIG. 53 is a view showing the positional relationship between the covering film and the inner frame in the prosthetic aortic valve device;

FIG. 54a is a front view of the turned-over frame of the prosthetic aortic valve device in one embodiment;

FIG. 54b is a left side view of the prosthetic aortic valve device of FIG. 54a;

FIG. 54c is a perspective view of the prosthetic aortic valve device of FIG. 54b;

FIGS. 55a to 55d are respectively a front view, a left side view, a perspective view and a top view of the inner frame of the prosthetic aortic valve device in a compressed configuration;

FIGS. 56a-56d are respectively a front view, a left side view, a perspective view and a top view of the inner frame of the prosthetic aortic valve device in an expanded configuration;

FIGS. 57a to 57d are respectively a front view, a left side view, a perspective view and a top view of the turned-over inner frame of the prosthetic aortic valve device in an expanded configuration;

FIG. 58a is a top view of the prosthetic aortic valve device in a released configuration in one embodiment;

FIG. 58b is a view of the prosthetic aortic valve device and the aortic valve prior to circumferential positioning therebetween;

FIG. 58c is a view of the prosthetic aortic valve device and the aortic valve after circumferential positioning therebetween;

FIG. 58d is a view showing the engagement between the prosthetic aortic valve device and the aortic valve;

FIG. 59a illustrates the guiding member after placement in the valvular sinus and before engagement;

FIG. 59b illustrates the deformation of the guiding member at the initial stage of release;

FIG. 59c illustrates the guiding member being positioned in the valvular sinus after released;

FIGS. 60a to 60c are respectively a front view, a perspective view and a right side view of a guiding member in a loaded configuration;

FIGS. 61a to 61c are respectively a front view, a perspective view and a right side view of the guiding member in a transition configuration;

FIG. 62 is a front view of the guiding member in a released configuration (with the root turned over);

FIG. 63 is a perspective view of the guiding member of FIG. 62;

FIG. 64 is an enlarged view of part C of FIG. 62b;

FIG. 65 is an enlarged view of part B of FIG. 60b;

FIG. 66 is a top view of FIG. 47a;

FIG. 67 is a view of connected bars of a root;

FIG. 68 is a view of parallel bars of a root;

FIG. 69 is an enlarged view of part A of FIG. 52;

FIG. 70a is a perspective view of a guiding member of a prosthetic aortic valve device in one embodiment before preset;

FIG. 70b is a perspective view of the bars of the root of the guiding members of FIG. 70a being close to each other (transition configuration);

FIG. 70c is a perspective view of the bars of the root of the guiding members of FIG. 70b being away from each other (released configuration);

FIG. 71 is a perspective view showing the distribution of developing markers in a prosthetic aortic valve device;

FIG. 72 is a view of a delivery system according to an embodiment of the present application;

FIG. 73 is a view of the distal section of the delivery system of FIG. 72 loaded with a prosthetic aortic valve device;

FIG. 74 is a view of the tube of the prosthetic aortic valve device;

FIG. 75 is a view of another tube of the prosthetic aortic valve device;

FIG. 76 is a partial view of another form of control handle in the delivery system;

FIG. 77 is a partial view of another form of control handle in the delivery system;

FIG. 78 is a partial view of another form of control handle in the delivery system;

FIGS. 79a-80b are views showing the release process of the prosthetic aortic valve device;

FIG. 81 is a flow chart of a method for using an interventional system according to an embodiment of the present application.

FIG. 82 is a view of the prosthetic aortic valve device in a loaded configuration (leaflets are not shown).

FIG. 83 is a view of the prosthetic aortic valve device of FIG. 82 in a transition configuration;

FIG. 84 is a view of the prosthetic aortic valve device of FIG. 83 in a released configuration;

FIG. 85a is a view of a prosthetic aortic valve device according to an embodiment;

FIGS. 85b-85c are views of a prosthetic aortic valve device according to an embodiment;

FIG. 85d is a perspective view of a prosthetic aortic valve device according to an embodiment;

FIG. 86 is a perspective view of a prosthetic aortic valve device according to another embodiment;

FIGS. 87a to 87c show different configurations of the guiding member of the prosthetic aortic valve device;

FIG. 88a is a view of the prosthetic aortic valve device and the aortic valve prior to circumferential positioning therebetween;

FIG. 88b is a view of the prosthetic aortic valve device and the aortic valve after circumferential positioning therebetween;

FIG. 89a is a view of a guiding member in an embodiment;

FIG. 89b is an enlarged view of part C of FIG. 89a;

FIG. 89c is a view of the prosthetic aortic valve device in a transition configuration;

FIG. 89d is a flattened view of the prosthetic aortic valve device;

FIG. 90 shows the engagement between the guiding member and the valvular sinus after the former is inserted into the later;

FIG. 91a is a view of wings of the guiding members according to one spatial configuration;

FIG. 91b is a view of wings of the guiding members according to another spatial configuration

FIG. 92a is a flattened view of the one-piece guiding member;

FIG. 92b is a flattened view of the one-piece guiding member in another form;

FIG. 92c is a flattened view of the one-piece guiding member in another form;

FIG. 93a is a perspective view showing the distribution of developing markers in a prosthetic aortic valve device;

FIG. 93b is a perspective view showing the distribution of developing markers in a prosthetic aortic valve device in another form;

FIG. 94 is a perspective view of a prosthetic aortic valve device of another embodiment;

FIGS. 95 and 96 are perspective views showing spacing regions of a prosthetic aortic valve device in different configurations.

### LIST OF REFERENCE NUMERALS

100, prosthetic heart valve device; 1000, prosthetic aortic valve device; 101, inflow end; 102, outflow end; 103, inner frame; 104, connecting post; 1041, fifth bar; 1042, sixth bar; 110, frame; 111, spacing region; 1112, eyelet; 114, commissure region; 115, first collar; 116, cell; 117, second collar; 120, clipping arm; 121, fixed end; 1221, rounded structure; 123, free end; 127, connecting portion; 129, projection area; 132, commissure post, 141, leg, 142, second frame arm, 146, apex, 160, first angled space, 168, slot, 173, developing point;
200, leaflet; 201, native leaflet; 204, valvular sinus; 211, joining region; 220, covering film; 221, inner covering film; 223, outer covering film;
301, blood flow channel; 310, connecting portion; 3101, developing hole; 311, positioning structure; 3120, sleeve; 313, rigid portion; 314, flexible portion; 315, connecting member; 321, unit; 330, wave structure; 340, connecting ring; 341, a connecting region, 342, a second angled space, 343, a first position, 344, a second position, 345, a flexible member;
400, delivery system; 404, support device; 405, outer sheath; 406, loading zone; 407, control handle; 410, support; 411, sliding groove; 420, movable base; 430, driving sleeve; 440, rotatable seat; 441, planetary carrier; 442, planetary gear; 443, ring gear; 444, planetary input shaft; 445, planetary output shaft; 451, worm wheel; 452, worm; 453, transmission sleeve; 454, support base; 461, first gear; 462, second gear; 463, transmission sleeve; 464, support base;
530, guiding member; 531, wing; 531a, wing; 531b, wing; 531c, wing; 53 Id, wing; 53 le, wing; 53 If, wing; 532, root; 532a, root; 532b, root; 5321, first bar; 5322, second bar; 5323, first binding eyelet; 5324, first connection point; 5325, second connection point; 5326, third connection point; 5327, first plane; 5311, first wing; 5312, second wing; 534, free end; 5341, wave structure; 535, branched structure; 5351, third bar; 5352, fourth bar; 5353, slot; 5354, second binding eyelet; 5355, fourth connection point; 5356, second plane; 536, free end; 5361, seventh bar; 5362, eighth bar; 537, restricting structures; 538, first portion; 539, second portion; 550, developing marker; 550a, developing marker; 550b, developing marker; 550c, developing marker; 551, eyelet;
600, balloon device; 610, tube; 6101, outermost layer; 6102, middle layer; 6103, innermost layer; 620, guide head; 630, balloon;
900, human heart; 910, aorta; 911, right coronary artery trunk; 912, left coronary artery trunk.

### DESCRIPTION OF THE EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

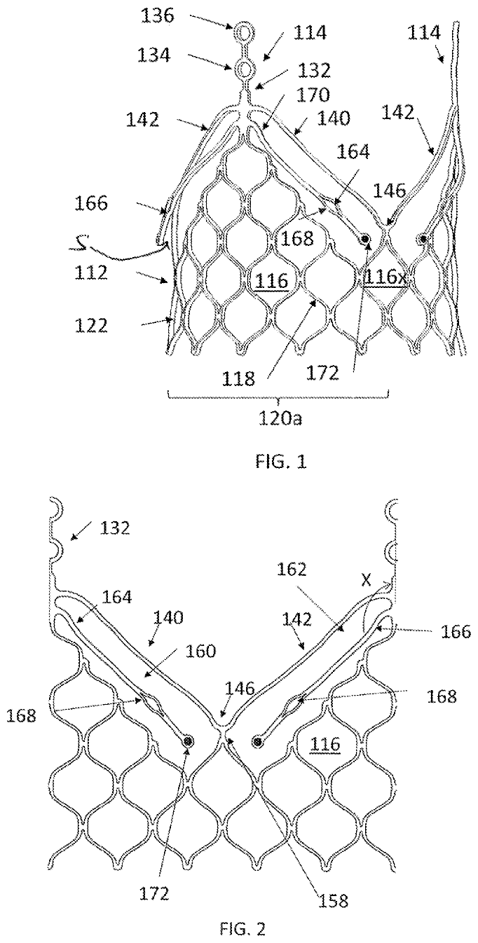

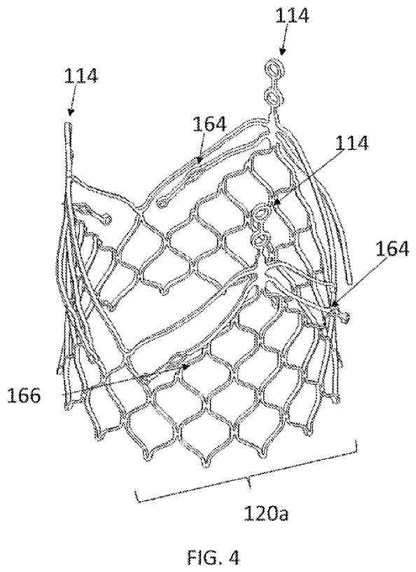

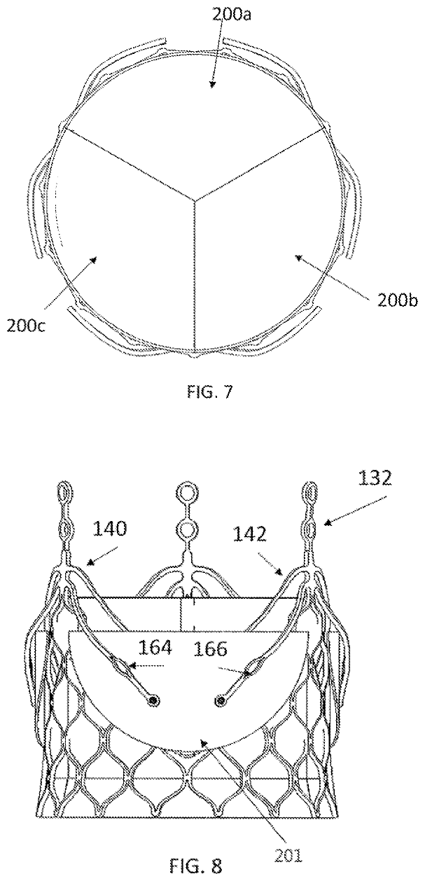

FIGS. 1-9 illustrate a prosthetic heart valve device 100 according to the present invention. The device 100 has a frame 110 that is defined by an annular body 112 that includes three commissure regions 114. The body 112 is defined by an arrangement of cells 116. Each cell 116 can be defined by four struts 118 to form any desired shape. FIGS. 1-9 show the struts 118 being curved to form a tear-drop shaped cell, although any other configuration (e.g., four straight struts to form a diamond-shaped cell) can also be employed.

Referring first to FIGS. 1-4, the body 112 can be configured with three cell regions 120a, 120b and 120c that form an annular band. Each cell region 120a, 120b and 120c consists of cells, having a first row 122 of cells 116, a second row 124 of cells 116, a third row 126 of cells 116, a fourth row 128 of cells 116 and a fifth row of cells 130. The first row 122 defines the distal (or inflow) end of the frame 110 and has the largest number (e.g., five in this embodiment) of cells 116. The second row 124 is immediately proximal of, and staggered from, the first row 122 and the same or next largest number (e.g., five in this embodiment) of cells 116. The third row 126 is immediately proximal of, and staggered from, the second row 124 and the next largest number (e.g., three in this embodiment) of cells 116. The fourth row 128 is immediately proximal of, and staggered from, the third row 126 and the next largest number (e.g., two in this embodiment) of cells 116. The fifth row 130 is immediately proximal of, and staggered from, the fourth row 124 and the smallest number (e.g., one in this embodiment) of cells 116. The fifth row 130 is also the proximal-most (outflow) row of cells 116, and each cell 116 in the fifth row 130 support a respective commissure post 132.

As best shown in FIG. 1, the distal (inflow) end of the body 112 can be slightly flared so that the increased diameter at the distal (inflow) end can be used to better secure the frame 110 at the native annulus. In particular, the cells 116 in the first row 122 can be shape-set to be flared and define a larger diameter than the next row 124 of cells 116.

Each commissure region 114 includes a connection portion, and in this embodiment, the connection portion is configured as a commissure post 132 extending from the distal-most row of cells 116. Each commissure post 132 includes at least one eyelet 134 that extends from the top (proximal end) of the post 132, and in this embodiment, there is also a second eyelet 136. In case where a plurality of eyelets 136 is provided, the plurality of eyelets 136 can be arranged along the length of the respective commissure post 132, although other arrangements can also be employed.

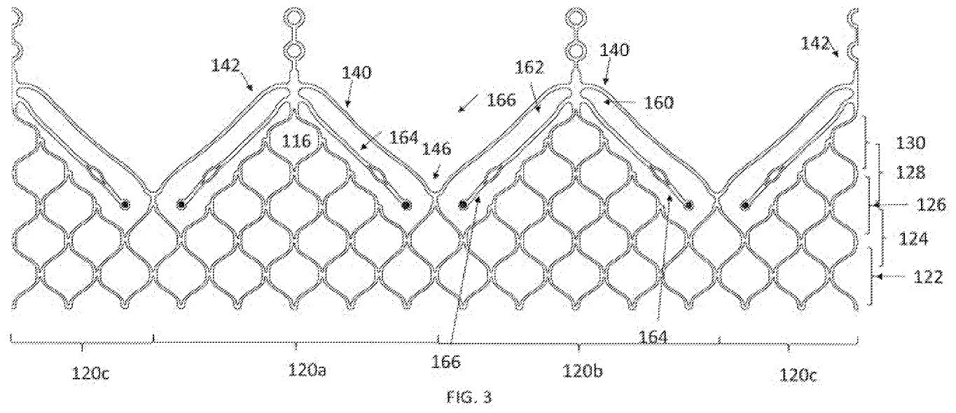

A first frame arm 140 and a second frame arm 142 extend from opposite sides of each post 132. Each first arm 140 from one post 132 is connected at a distal-facing apex 146 with the second arm 142 from an adjacent post 132. Each apex 146 is joined or connected at a joint 158 with an apex of a cell 116x in row 124. Each arm 140 and 142 can be straight or wavy (as shown, with different curved regions along the arm) or curved. The first frame arm 140 is connected with the frame 110 at a joint that is closer to the distal (or inflow) end than the eyelet 134. Each arm 140 and 142 is formed as a single rod of deformable mesh band. As shown in FIG. 3, the angle between each arm 140 and 142 and the axis of the frame ranges from 30 degrees to 85 degrees.

In addition, a first angled space 160 is defined by each first arm 140 and the corresponding cell region 120a, 120b or 120c, and a second angled space 162 is defined by each second arm 142 and the corresponding cell region 120a, 120b or 120c. The first angled space 160 is inclined towards the distal (or inflow) end away from the corresponding post 132. The first arm 140 and the second arm 142 generally form a V-shape between two adjacent posts 132, and the first arm 140 and the second arm 142 can be symmetrically disposed at opposite sides of the imaginary apex of the V-shape.

A first clipping arm 164 and a second clipping arm 166 extend from opposite sides of each connection portion for connecting the clipping arms 164, 166 and the frame (i.e., here it would be the post 132) from a location between the first arm 140 and the second arm 142, respectively, and the corresponding distal-most row of cells 116. Alternatively, the first clipping arm 164 and the second clipping arm 166 can extend from opposite sides of the same row of cells 116. Preferably, the circumferential span between the first clipping arm 164 and the second clipping arm 166 is small. More preferably, the axial span between the first clipping arm 164 and the second clipping arm 166 is also small. In the present embodiment, the joint of the first clipping arm 164 with the corresponding post is adjacent to the joint of the second clipping arm 166 with the same corresponding post. Preferable, the first clipping arm 164 and the second clipping arm 166 can be symmetrically arranged at opposite sides of the corresponding post 132. The joint of the first arm 140 and the corresponding post is closer to the proximal (or outflow) end than the joint of the first clipping arm 164 and the second clipping arm 166. In other embodiments, additional clipping arms 164, 166 can be provided on any post 132, in which case all the clipping arms 164, 166 on the same side of the post 132 can be considered as being of one group of clipping arms. The ends of the clipping arms in one group for connecting with the post can be joined at the same position or adjacent to each other, and the free ends of the clipping arms in one group can be connected together. In some embodiments, each clipping arm can be formed as a deformable mesh band.

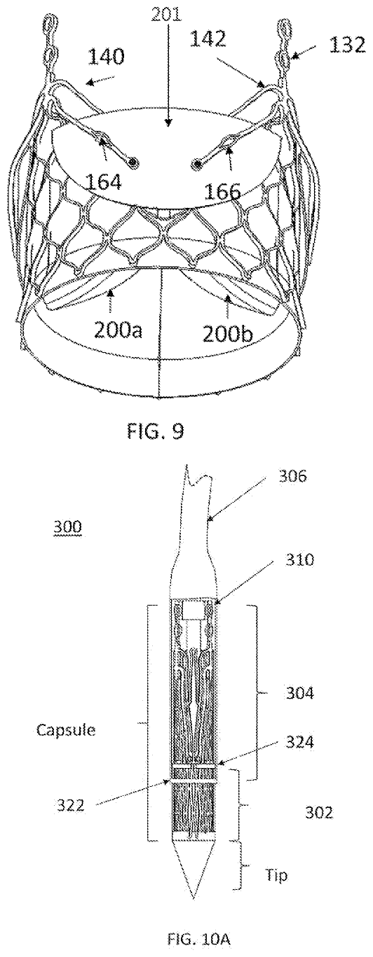

The first angled space 160 can be considered as a hollowed area of the frame 110. Before the clipping arms 164 and 166 are released and expanded, the clipping arms 164 and 166 can be located within the respective hollowed areas, which avoids providing the frame 100 with a large outer diameter by avoiding a radial overlap during loading of the device 100. Each clipping arm 164, 166 defines an angle X between the respective clipping arm 164, 166 and the respective commissure post 132. See FIG. 2. This angle X ranges from 90 degrees to 180 degrees, and is preferably about 120 degrees. The clipping arms 164 and 166 function as beams, so the term “beam” is also used interchangeably herein with the term “clipping arm”, and are intended to have the same meanings. Each clipping arm 164 and 166 can include one or more slots 168 within the body of the arm 164 or 166, and each arm 164 and 166 has a first end 170 connected to the corresponding post 132, and a second end having an eyelet 172 that slightly enlarges the second end. Each first clipping arm 164 and second clipping arm 166 extends into the vicinity of the first angled space 160 and the second angled space 162, respectively, and functions to clip a portion of a native valve leaflet (see FIGS. 8 and 9) against the body 112. The clipping arms 164, 166 can be provided in one piece, cut from the same tubular structures as the body 112, or attached after cutting by various methods, such as suture attachment or laser welding.

Each slot 168 can be an open space in the body of the arm 164 or 166, and this open space can have any desired shape, including diamond shaped (as shown). As such, these slots 168 can be considered to be extender cells. The purpose of these extender cells 168 is to lengthen the arm 164 or 166 after the initial frame cutting. The extender cells 168 are designed in such a way that, prior to shape set, they are in the open configuration (i.e., struts are further apart) but after shape-set, they are in the closed configuration. By changing from the open to the closed configuration, the extender cells 168 foreshorten and cause the beams to elongate. This allows the frame 110 to be designed out of a single tubing and achieve the length required to allow the heart valve device 100 to sit high enough in the aortic annulus to minimize protrusion into the left ventricular outflow tract (LVOT) and thereby minimize risk of conduction system disturbance. Each arm 164 and 166 can have a plurality of extender cells 168 that are spaced-apart along the length of each arm 164, 166. As another alternative, the size of the slots 168 can be varied depending on use, application, and clinical requirements.

Each clipping arm 164 and 166 can have a length of about 18 mm, although the length could be adjusted based on clinical requirements. As shown in the figures, each arm 164 and 166 extends across most of the respective space 160 and 162. The arms 164 and 166 serve two purposes. First, each arm sits behind the native leaflet, and holds the native leaflet between the arm and the frame body. This allows the native leaflet to be used to improve in the sealing between the native anatomy and prosthesis. Additionally, a secondary clipping mechanism can be obtained by capturing the leaflet between adjacent arms. Second, the arms limit protrusion into the LVOT and thus minimize conduction system impact. The arms 164, 166 are deployed first and are placed such that the tips 172 seat inside the cusp. The tip location can be modulated by changing the length or angle of the arm 164, 166 with the commissure post. Thus, the tip 172 can be designed to be in the optimal location in relation to the inflow of the heart valve, approximately 4-8 mm from the distal most end of the frame 110, thereby limiting protrusion into the LVOT. In addition, the stiffness of the arms 164, 166 can be modulated by changing the thickness of the arms 164, 166 such that there is more or less flexing when contacting the native leaflet. The desired embodiment will seat the valve prosthesis within the native anatomy such that the protrusion of the frame 110 into the LVOT is minimized and reduces instances of PPI (permanent pacemaker implantation). For improved safety, the tips 172 can be provided as a rounded structure and/or covered with a protective layer which is preferably made of a biocompatible synthetic material of biomaterial.

The arms 164, 166 in the current embodiment are shape-set to a larger diameter than the body 112 of the frame 110 by approximately 4 mm. In other words, the outer diameter formed by tracing the tips 172 of all the arms 164, 166 can be equal to or larger than the outer diameter of the body 112 at the circumference location of the tips 172. This can be shown or represented by the space S in FIG. 1. This shape-setting configuration (in addition to the arms 164, 166 being free at one end) allows the arms 164, 166 to expand to a larger diameter during delivery. The larger diameter expansion increases the chances of capturing the native leaflets between the arms 164, 166 and the body 112 of the frame 110, thus increasing the likelihood of correct anatomical placement of the prosthetic device 100. As a non-limiting alternative, each clipping arm 164 and 166 can extend outwardly in a wavy pattern to further improve the clipping effect of the arm to the portion of the native valve leaflet against the body 112 without affecting the positioning in the human body.

An alternative is to provide varying spacing S of the tips 172 along the circumference of the frame 112. For example, the spacing S can be 4 mm at some tips 172, and 3 mm at other tips 172.

The frame 110 can be made of Nitinol or any other known self-expandable material having superelastic memory characteristics.

Even though the frame 110 is described hereinabove with specific reference to one specific embodiment, this is not intended to be limiting and it is also possible to configure the frame 110 differently.

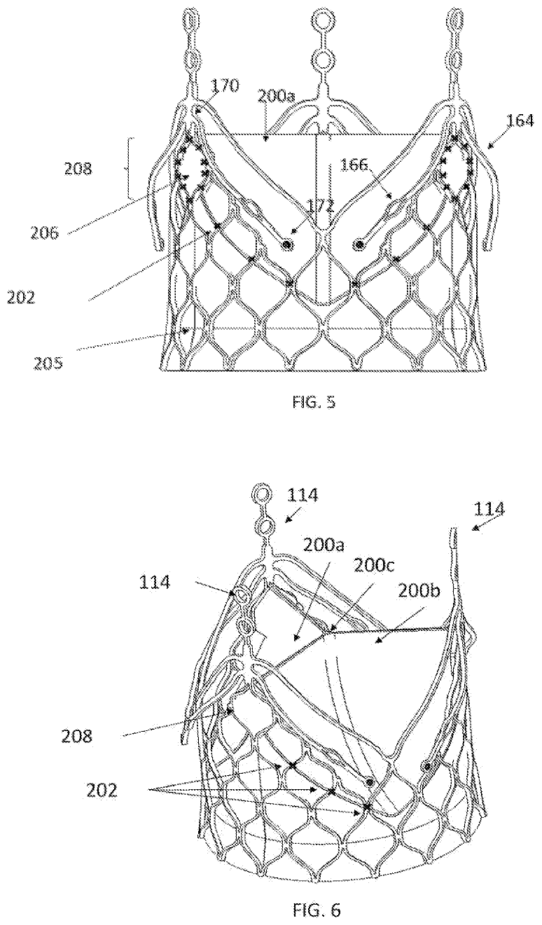

Referring now to FIGS. 5-7, the device 100 also has a set of prosthetic leaflets 200a, 200b and 200c that are configured as a conventional tricuspid (three-leaflet) valve. The leaflets 200a, 200b and 200c can be provided in any known desired prosthetic material, including a processed animal tissue such as porcine tissue and bovine tissue, or a synthetic material.

The three leaflets are attached together using a stitch line, and commissure tabs 206 are created by folding back the leaflet tabs and attaching to a cloth material. Commissure tab cloth material can be made from synthetic material (e.g., polyester) and aids in suture retention in attaching the tissue subassembly to the frame 110. Once formed, the leaflet subassembly is stitched to a skirt material 205. The skirt is similarly created from three separate components and stitched together. The skirt material 205 can be made from porcine or bovine tissue, or a synthetic material. Once this sub-assembly is created, the commissure tabs 206 are attached at locations 208 to the frame 110 at the proximal-most row (row 130) of cells 116 to form the commissure. In one embodiment, the seam line between each leaflet 200a, 200b, 200c and skirt material 205 is attached to the frame 110 using stitches (see attachment points 202) at the appropriate locations, although other attachment methods are also possible. Further, additional stitching is utilized to secure the skirt material to the frame 110 between the bottom of the frame 110 and leaflet attachment. An example of attachment points 202 are the points or locations where the leaflet edges are attached to the cells 116. The cells 116 in the row 130 will be utilized for commissure attachment, and the leaflets will be attached along a curved path following the shape of the leaflet through the plurality of cells shown in FIG. 6 as defined by the attachment points 202. The skirt material 205 is attached spanning the space from the leaflet attachment to the bottom of the frame.

The device 100 of the present invention provides a number of benefits over the existing transcatheter aortic valve implantation devices that are used for treating Al disease.

First, the frame 110 has a plurality of beams or clipping arms 164 and 166 that function as cantilever beam-like structures that are designed to clasp onto the native aortic leaflets securely, and to do so with ease. Specifically, during device implantation, the clipping arms 164, 166 are exposed from the delivery catheter first, and are positioned behind and/or around the native leaflets. Once the device 100 is fully deployed, the clipping arms 164, 166 will mechanically clasp onto the native leaflets, thus keeping the device 100 in place. The mechanical clasp force can be enhanced by shape-setting the arms in a configuration that are matched to act like clips.

Unlike the existing JenaValve or J-Valve devices, which position three large parabolic or “U” shaped arches behind the three native leaflets, the device 100 of the present invention uses six cantilever beams 164, 166 to clip onto the native leaflets. This provides two major benefits. First, the additional clipping arms or beams increase the likelihood of successfully capturing one or more native leaflets, thus making the procedure easier and safer. Second, successful clasping of the native leaflets can be accomplished in multiple ways, thus making the anchoring mechanism more secure and reliable. For example, the leaflet clasping can either be obtained by placing all arms 164, 166 behind the native leaflet and securing the native leaflet between the arms 164, 166 and the body 112; or alternatively by placing a plurality of arms 164, 166 behind the native leaflet and a plurality of arms 164, 166 in front of the native leaflet, thus clasping the native leaflet between adjacent arms 164, 166. The underlying benefit of having closely located clipping arms is that they are designed so that they can act as clips with respect to leaflet backing if all the arms 164, 166 are behind the native leaflets. In certain scenarios when some of the arms 164, 166 are not behind the native leaflets, the offset shape-setting has the closely located arm also clasp the native leaflet and provide better anchoring. For example, there can be a clinical situation where multiple clipping arms 164, 166 can be in front of the native leaflets. This positioning of the clipping arms in the front and the back of the native leaflets provides a better clasping action on the native leaflets compared to the existing devices, where the arches of those devices must be positioned behind the native leaflets.

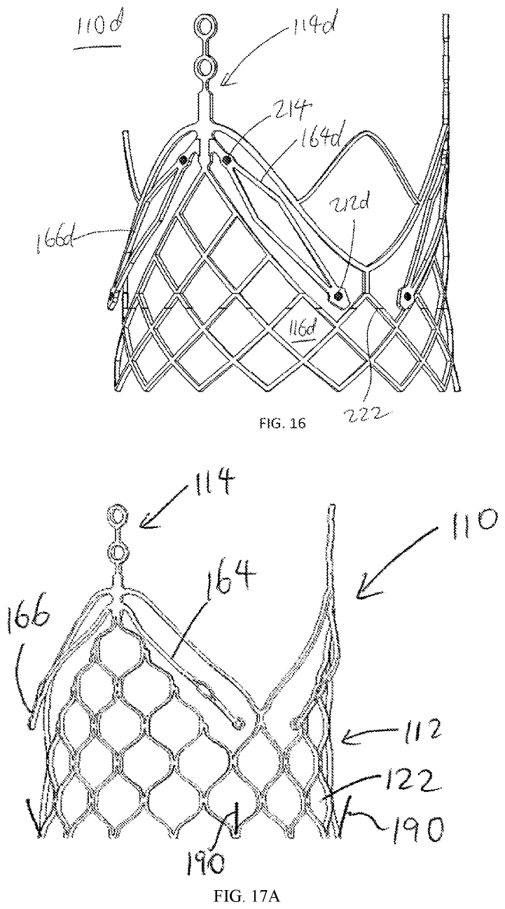

Second, the clipping arms 164, 166 also feature an atraumatic tip (the eyelet 172), which can be loaded with a radiopaque marker 212 for ease of visualization during implantation. There can also be an additional radiopaque marker 214 (see FIG. 16) located in the commissure regions 114 to allow for the comparison of movement between the eyelets 172 and the commissures. Based on the motion of the markers 212, 214 during deployment, these markers can assist in identifying which clipping arm is behind the leaflet and which clipping arm is in front of the leaflet. For example, the marker that is moving at the rhythm of the heartbeat generally can help illustrate that it is touching the nadir of the native leaflets. By having radiopaque markers on the clipping arms and commissure regions, the physician can easily ascertain that the clipping arms are located behind the native leaflets before full deployment of the device 100. This would make the procedure faster and safer.

Third, the frame 110 provides leaflet restraints or leaflet backing, which are long struts (i.e., the arms 140 and 142) emanating from the posts 132. These leaflet-restraint structures provide additional clasping of the native leaflets to the frame 110 while keeping the native leaflet trapped between the clipping arms 164, 166. These leaflet-restraint structures prevent the native leaflets from interfering with the prosthetic leaflets, and can also work with the clipping arms to clip onto the native leaflets.

Fourth, the frame 110 provides a closed cell design, which means that all struts 118 are connected to each other. Such a design allows for the device 100 to be re-sheathed as there are no open cells which would inhibit the catheter sheath from recovering the entirety of the frame 110 due to any struts from open cells catching the outer sheath 306.

FIGS. 8-11G illustrate how the device 100 is delivered to an aortic annulus and deployed at the aortic annulus. First, FIGS. 8 and 9 show native leaflets 201 clipped or sandwiched between the clipping arms 164, 166 and the body 112 of the frame 110 after the device 100 has been deployed at the aortic annulus.

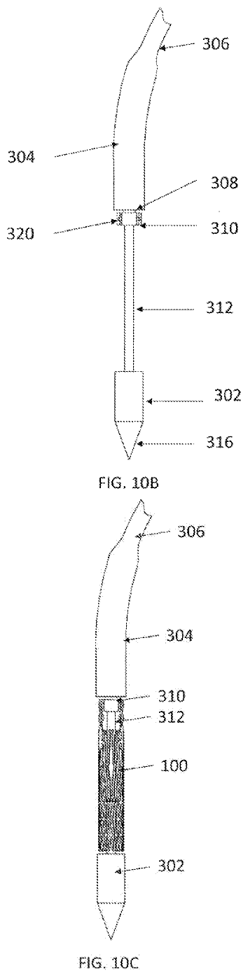

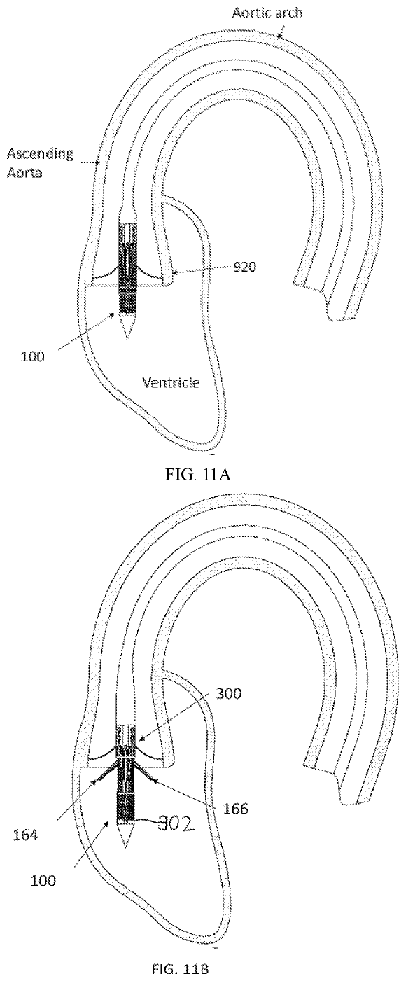

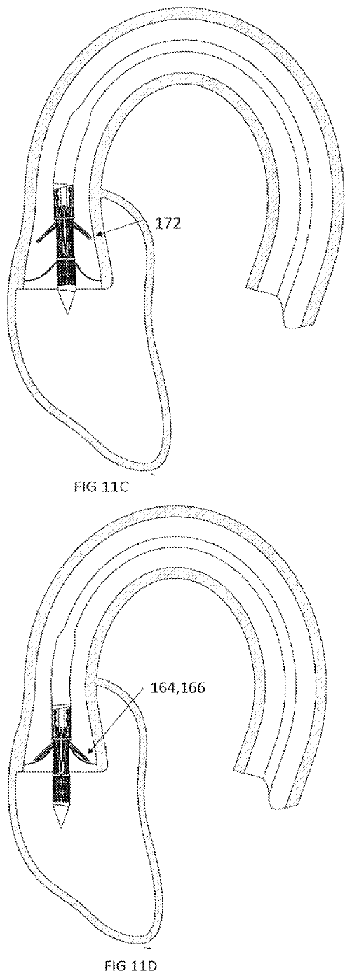

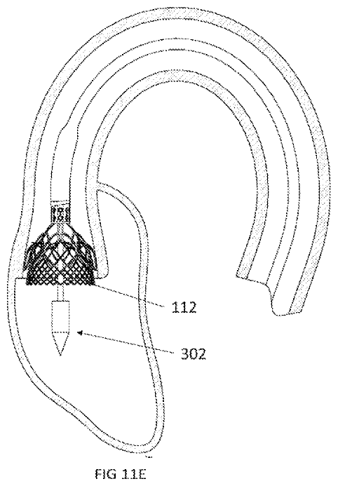

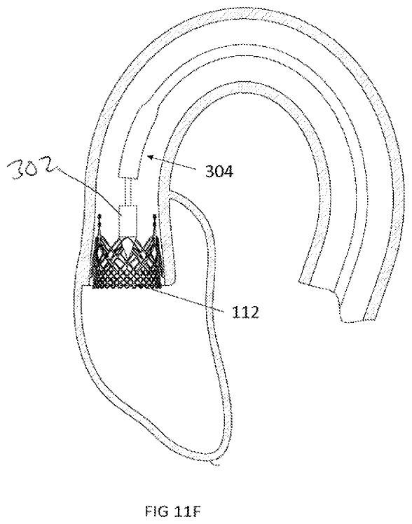

Referring now to FIGS. 10 and 11A-11Q the device 100 is first compressed and held in a delivery system 300. The delivery system 300 shown in FIG. 10 is simply one non-limiting example, and it has an outer sheath 306, an inner tube 308 with a dock 310 at its distal end, a distal sheath 302, and distal tip 316, that are mounted at the distal end of a shaft 312, and a proximal sheath 304. The proximal sheath 304 is mounted at the distal end of the outer sheath 306, and the inner tube 308 extends inside the lumen of the outer sheath 306. The dock 310 has protrusions 320 that are adapted to be clipped inside the eyelets 134/136 to hold or retain the frame 110 inside the delivery system 300. The shaft 312 is slidably retained inside the bore of the inner tube 308.

When the device 100 is crimped or compressed inside the delivery system 300 (see FIGS. 10A and 10C), the commissures 114 are adjacent to the dock 310 with the eyelets 134/136 coupled to the protrusions 320. The compressed device 100 surrounds the shaft 312, with the proximal sheath 304 covering most of the length of the device 100. The distal sheath 302 covers a small length of the distal end of the device 100, with the distal end of the proximal sheath 304 overlapping and covering the proximal end of the distal sheath 302. In particular, the proximal sheath 304 can have a band 322 at its distal-most end, and the distal sheath 302 can have a band 324 at its proximal-most end. The bands 322 and 324 can contain a radiopaque marker to allow for visualization by the clinician during the deployment procedure. The device 100 is contained in its entirety inside a capsule defined by the distal sheath 302 and the proximal sheath 304.

Referring to FIG. 11 A, the catheter 300 with the device 100 retained therein is introduced via a puncture wound at upper thigh region through the femoral artery, and advanced through the aortic arch and the ascending aorta to the location of the aortic annulus 920, and a portion of the capsule passes through the aortic annulus into the ventricle. Next, in FIG. 11B, the proximal sheath 304 is retracted so that the distal (eyelet 172) ends of the clipping arms 164, 166 are exposed in the ventricle. In the next step (see FIG. 11C), the device 100 is retracted by retracting the delivery system 300 so that the distal (eyelet 172) ends of the clipping arms 164, 166 have cleared the aortic annulus and are now positioned inside the aortic fixed end. With the distal ends of the clipping arms 172 positioned above the native aortic valve, the device 100 is advanced distally until the clipping arms 164, 166 drop into the cusps of the native leaflets. See FIG. 11D. Since there are three of each clipping arm 164, 166, there are a total of six clipping arms which allow the six clipping arms to be spaced-apart within the native leaflets in the aortic annulus. Next, the distal sheath 302 is advanced to expose the distal end of the device 100, so that the distal end of the device 100 can be deployed. This is shown in FIG. HE where the body 112 of the frame 110 is being expanded. At this point, the device 100 is secured at the aortic annulus. The proximal sheath 304 is then further retracted to deploy the commissure regions 114. See FIG. 1 IF. Finally, the distal tip sheath 302 is retracted into the proximal tip sheath 304, and the entire delivery system 300 is withdrawn from the human body. See FIG. 11G.

The method steps described in connection with FIGS. 11A-11G provide an advantageous way to adjust the position of the body 112 of the frame 110. Specifically, the distal sheath 302 is provided to retain the body 112 in its compressed configuration while the clipping arms 164, 166 are being released and positioned inside the cusps of the native leaflets. After the clipping arms 164, 166 have been properly positioned, the clinician can then maneuver the frame 110 in its compressed configuration so that the body 112 can be accurately positioned in the aortic annulus before it is released. The bands 322, 324 can help the clinician during this positioning step.

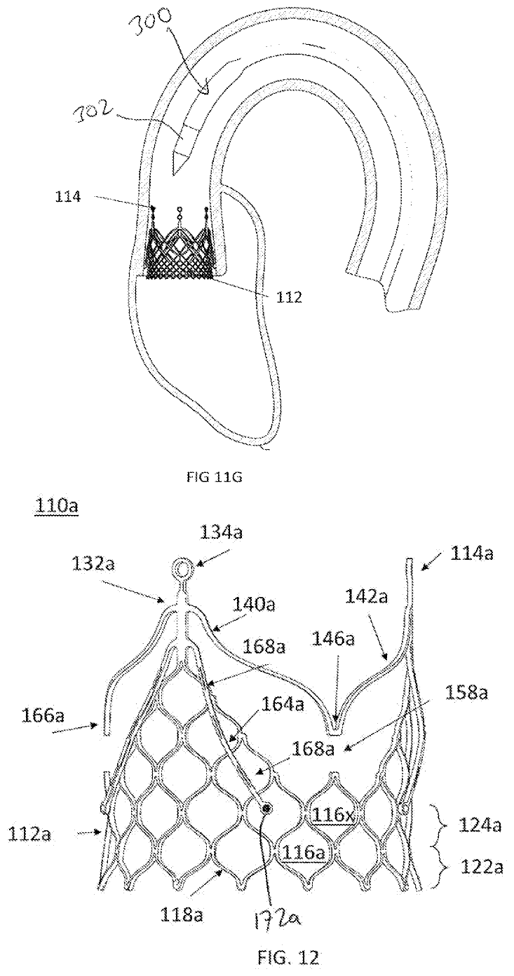

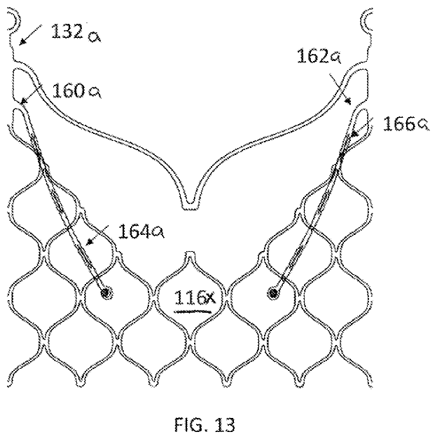

FIGS. 12-13 illustrate a second embodiment for the frame 110 of the present invention. The frame 110a in FIGS. 12 and 13 is very similar to the frame 110 in FIGS. 1-9, so the same numerals used in FIGS. 1-9 will be used in FIG. 12 to designate the same elements except that an “a” is added to the numerals in FIG. 12. The frame 110a differs from the frame 110 in three ways.

First, the apex 146a is not connected to, or joined with, the cell 116x of the body 112a. Therefore, a space 158a (instead of the joint 158) is defined between the apex 146a and the apex of the cell 116x in row 124a. Disconnecting the apex 146a and cell 116x, and creating a space 158a, allows the struts 140a and 142a of the leaflet backing to shape-set more naturally and reduces stress on the frame 110a during shape-setting of the frame 110a.

Second, each clipping arm 164a and 166a can have more than one slots 168a that are spaced apart along the length of each clipping arm 164a and 166a. The inclusion of additional slots 168a allows for additional length to be obtained in the clipping arms 164a, 166a. The length of each clipping arm 164a, 166a is one factor determining the location of the tips 172a and thus, the placement of the device 100 in the native anatomy. In other words, by adding additional slots 168a, the length of each clipping arm 164a, 166a extends, thereby allowing the device 100 to sit higher in the native anatomy. Additionally, the clipping arms can be angled at a more obtuse angle to allow the tips 172a to sit closer to the inflow(distal) end of the frame 11 ©. Reduction of the distance from the tips 172a to the inflow(distal) end of the frame 110 allows the device 100 to sit higher in the native anatomy, thus reducing the chance of conduction system disturbance, and thus PPI.

Third, only one eyelet 134a is provided at the commissure posts 132a, with the second eyelet 136 being omitted. The device 100 can be provided with a single or double eyelet depending on the dock 310. A double-eyelet (134+136) configuration may provide a more secure locking with the dock 310 in the delivery system 300, while a single eyelet 134a will reduce the overall height of the device 100. Additionally, a double-eyelet configuration may provide a mechanism for additional modulation of the arm position in-vivo.

In addition, although the present invention illustrates the use of eyelets to clip the protrusions 320 inside the dock 310, other alternatives to the eyelets can be provided in the posts 132 to accomplish the same function. As one example, a key structure can be used.

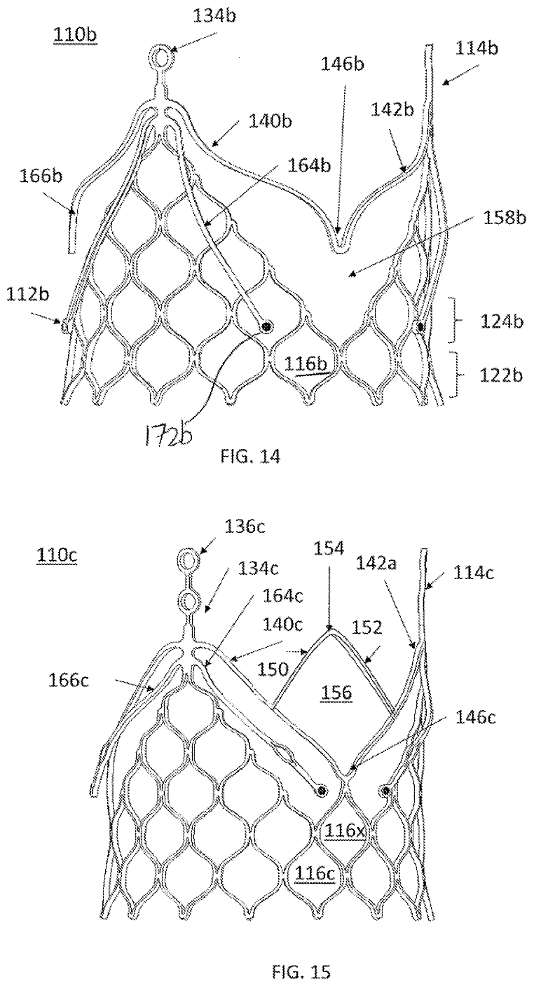

FIG. 14 illustrates a third embodiment for the frame 110 of the present invention. The frame 110b in FIG. 14 is very similar to the frame 110a in FIGS. 12-13, so the same numerals used in FIGS. 12-13 will be used in FIG. 14 to designate the same elements except that a “b” is added to the numerals in FIG. 14. The frame 110b differs from the frame 110a primarily in that the cell 116x is completely omitted, so that the space or distance 158b is larger than the space 158a, and also the length of the arms 164b and 166b are slightly increased. Eliminating cell 116x allows for the additional beam length to be added to the frame 110. This additional beam length allows the device 100 to sit higher in the native anatomy, and reduce the risk of PPI. This can be seen in FIG. 14 where the position of the tip 172b is lower on the frame 110b when compared to the other embodiments.

FIG. 15 illustrates a fourth embodiment for the frame 110 of the present invention. The frame 110c in FIG. 15 is very similar to the frame 110 in FIGS. 1-9, so the same numerals used in FIGS. 1-9 will be used in FIG. 15 to designate the same elements except that a “c” is added to the numerals in FIG. 15. In this embodiment, a curved bridge 148 can connect each set of first and second arms 140c and 142c at about their midsections. Each bridge 148 has a first leg 150 extending from a first arm 140c and a second leg 152 extending from a second arm 142c, and the two legs 150 and 152 connect at a proximal-facing apex 154. Each set of first and second arms 140c, 142c and legs 150 and 152 define a generally diamond-shaped space 156. As an alternative of the curved bridge 148, a deformable meshed structure defined by an arrangement of cells can also be provided to connect each set of first and second arms 140c and 142c at about their midsections.

FIG. 16. illustrates a fifth embodiment for the frame 110 of the present invention. The frame HOd in FIG. 16. is very similar to the frames 110 and 110c in FIGS. 1-9 and 15 so the same numerals used in FIGS. 1-9 and 15 will be used in FIG. 16 to designate the same elements except that a “d” is added to the numerals in FIG. 16. In this embodiment, an additional marker element is added to the connecting end of the clipping arms 164d, 166d. This allows for additional visibility under fluoroscopy in a clinical situation to better enable correct anatomical placement of the device. Based on the motion of the markers 212d, 214 during deployment, these two markers can assist in identifying which clipping arm 164d, 166d is behind the leaflet and which clipping arm is in front of the leaflet. For example, the marker that is moving at the rhythm of the heartbeat generally can help illustrate that it is touching the nadir of the native leaflets. By having radiopaque markers on the clipping arms and commissure regions 114d, the physician can easily ascertain that the clipping arms 164d, 166d, are located behind the native leaflets before full deployment of the device 100. This would make the procedure faster and safer.

Reviewing and comparing the embodiments in FIGS. 1 and 12-16 will illustrate an important aspect of the present invention in that the free ends (i.e., tips 172) of the clipping arms 164, 166 are positioned along a circumferential line (e.g., see 222 in FIG. 16) of the frame 110 which is closer to the inflow (distal) end of the frame 110 than to the outflow (proximal) end of the frame 110. The inflow end can be defined by the circumferential line defined by the distal-facing apices of the cells 116 in the first row 122, while the outflow end can be defined by circumferential line defined by the proximal-facing apices of the cells 116 in the fifth row 130.

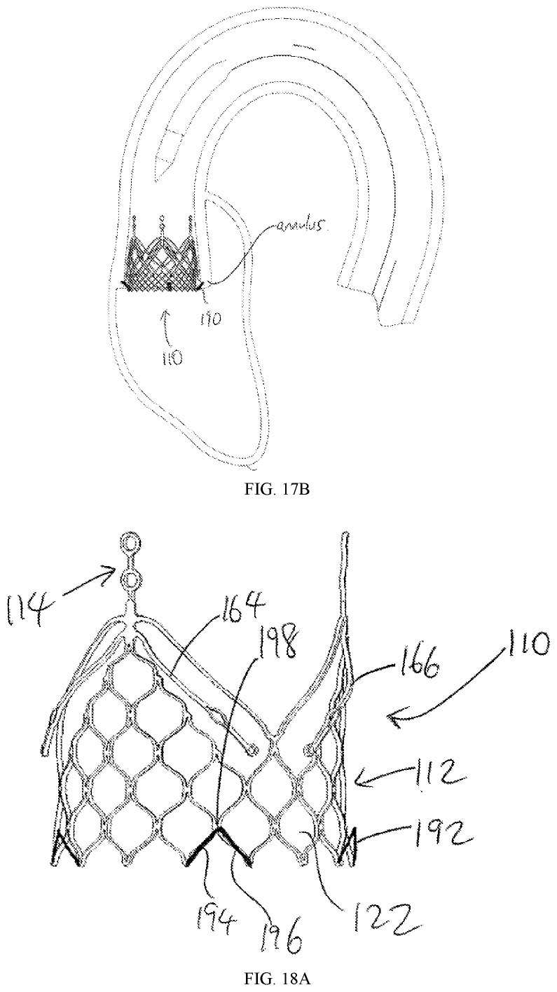

As described in connection with FIG. 1, the distal (inflow) end of the body 112 can be flared to provide a mechanism for securing the frame 110 at the native annulus. FIGS. 17A, 17B, 18A and 18B illustrate two other embodiments that utilize different mechanisms for securing the frame 110 at the native annulus.

FIG. 17A shows the same frame 110 as in FIG. 1, but with an everted strut 190 extending from the distal-most apex of some cells 116 in the row 122. These everted struts 190 can be shape-set to have one end connected to the distal-most apex of some cells 116 in the row 122, and an opposite free end which extends at an angle away from the body 112. FIG. 17B shows the location of the struts 110 when the frame 110 is secured at the native annulus.

FIG. 17B shows the same frame 110 as in FIG. 1, but with an everted cell 192 extending from the distal-most apex of some cells 116 in the row 122. Each everted cell 192 has two struts 194 and 196 that are connected to form an everted apex 198. These everted cells 192 can be shape-set to have one end of each strut 194, 196 connected to adjacent distal-most apices of some cells 116 in the row 122, and the opposite everted apex 198 extends at an angle away from the body 112. The cells 192 can even formed by removing certain cells 116 from the distal-most row 120 of cells. FIG. 18B shows the location of the cells 192 when the frame 110 is secured at the native annulus.

The dimensions and locations of the everted strut 190 and the everted cell 192 can be adjusted depending on the desired application. For example, the lengths of the struts 190, 194, 196 can be varied, and these struts 190, 194, 196 can even be curved. As another example, an everted strut 190 can be provided on any number of apices, or in any arrangement. For example, everted struts 190 can be provided on alternating apices. Also, the struts 194, 196 of the everted cells 192 do not need to extend from adjacent apices, but can extend from two separate apices that are separated by one apex.

While the description above refers to particular embodiments of the present invention, it will be understood that many modifications may be made without departing from the spirit thereof. The accompanying claims are intended to cover such modifications as would fall within the true scope and spirit of the present invention.

The frame of the prosthetic heart valve device and the prosthetic heart valve device hereinafter have different configurations in different applications, which mainly include an inner frame and a clipping arm, wherein the inner frame has relative compressed configuration and expanded configuration, and the clipping arm has corresponding loaded configuration and released configuration. Unless otherwise specified, the description related to the proportional relationship of the parts of the frame and the structure of the frame in the released configuration refers to the free condition of the frame outside the human body without force from surrounding tissue.

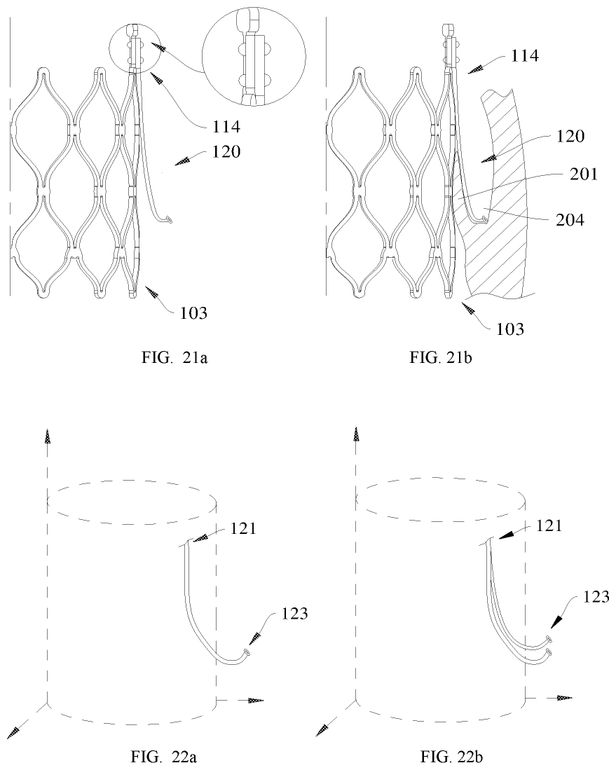

Referring to FIGS. 19 to 31, the present application discloses a frame 110 for a prosthetic heart valve device, including:
an inner frame 103 having a meshed cylindrical structure, which has relative compressed configuration and expanded configuration depending on the radial deformation, and a support device (e.g., a balloon) for driving the inner frame 103 to transform into the expanded configuration can be placed within the inner frame 103; and
a plurality of groups of clipping arms 120 located at an outer periphery of the inner frame 103 and spaced apart from each other in the circumference of the frame 110, each clipping arm 120 having opposite fixed end 121 and free end 123, the fixed end 121 being directly or indirectly connected with the inner frame 103, and the fixed ends 121 of the clipping arms 120 in the same group are adjacent to each other, and wherein the clipping arm 120 is made of memory material and has configurations of
a loaded configuration, in which the inner frame 103 assumes the compressed configuration, and the clipping arms 120 contact the inner frame 103; and
a released configuration, in which the inner frame 103 assumes the expanded configuration, the free end 123 of each clipping arm 120 expands radially outward, with a space defined between the free end 123 of each clipping arm 120 and the inner frame 103 to allow entry of the native leaflet 201, wherein the free ends 123 of at least two clipping arms 120 in the same group tend to extend away from each other, and the free ends 123 of at least two clipping arms 120 in adjacent groups tend to extend close to each other.

The meshed cylindrical structure of the inner frame 103 has an axis and a circumferential direction around the axis. The two axial ends of the meshed cylindrical structure are respectively configured as an inflow end 101 and an outflow end 102. The interior of the meshed cylindrical structure is configured as a blood flow channel 301, and the support device which can be placed within the inner frame 103 is configured to be located in the blood flow channel 301.

The frame 110 for the prosthetic heart valve device according to the present application is structurally improved, which improves the positioning of the prosthetic heart valve device for treating a pure aortic valve insufficiency disease, with the advantage of high assembly efficiency, convenient deployment and positioning, long-term stability and high durability, and having positive impact on the application of minimally invasive transcatheter aortic valve implantation devices for treating aortic valve insufficiency.

The clipping arms 120 are provided separately so as to avoid the positioning failure caused by an individual clipping arm(s) 120 which cannot be located in the valvular sinus. Taking the tricuspid valve as an example, three groups of clipping arms 120 are provided. The free ends 123 of at least two clipping arms 120 in each group tend to extend away from each other, which greatly increases the available anchor points, while the free ends 123 of at least two clipping arms 120 in the adjacent two groups tend to extend close to each, which conforms the anatomic structure of the valvular sinus. Furthermore, the clipping arms 120 which are made of a memory material can be self-expanded when they are released, while the inner frame 103 can be expanded with a support device (e.g., a balloon). Different expansion methods provide a more practical two-step releasing process. That is, the clipping arms 120 are released in the first step, and after the clipping arms 120 are positioned in place, the inner frame 103 is released in the second step, so that the inner frame 103 and the clipping arms 120 at the inner and outer sides of the leaflet 200, respectively, cooperate with each other. In the case where the inner frame 103 and the clipping arms 120 are both self-expanded, the delivery device would be more complicated and needs two sheaths connected in series or one surrounded by another so as to release the inner frame 103 and the clipping arms 120 in different steps, respectively, having more movable components and further reducing the compliance.

A specific portion can be provided by the inner frame 103 for connecting with the fixed end 121 of the clipping arm 120. Referring to FIG. 19, the inner frame 103 is provided with at least two commissure regions 114 spaced apart in the circumferential direction, and the fixed ends 121 of the clipping arms 120 in the same group are connected to a corresponding commissure region 114. Specifically, the inner frame 103 is provided with a plurality of commissure regions 114 adjacent to the outflow end 102, and the fixed ends 121 of the clipping arms 120 in the same group are connected to the corresponding commissure region 114. As shown in the figures, the number of the commissure regions 114 is preferably n, where n is the number of the leaflets 200 configured to be loaded in the frame 110. More specifically, the edge of the inner frame 103 at the outflow end 102 has a structure with peaks and valleys, and the commissure regions 114 are located at the peaks (which protrude towards the outflow end 102). In another aspect, the axial length of the inner frame 103 varies in the circumferential direction of the inner frame 103, and gradually shortens as far away from the commissure region 114. Further, as shown in FIG. 25, in the axial direction of the inner frame 103, the inner frame 103 has a plurality of rows of cells, including N rows of cells that respectively extend continuously in the circumferential direction adjacent the inflow end 101, and the remaining rows of cells respectively extend discontinuously in the circumferential direction, where N is 1, 2 or 3. As can be seen from the figure, in the circumferentially discontinuously extending rows of cells, the distance between the cells spaced from each other in the same row is larger as it is closer to the outflow end 102.

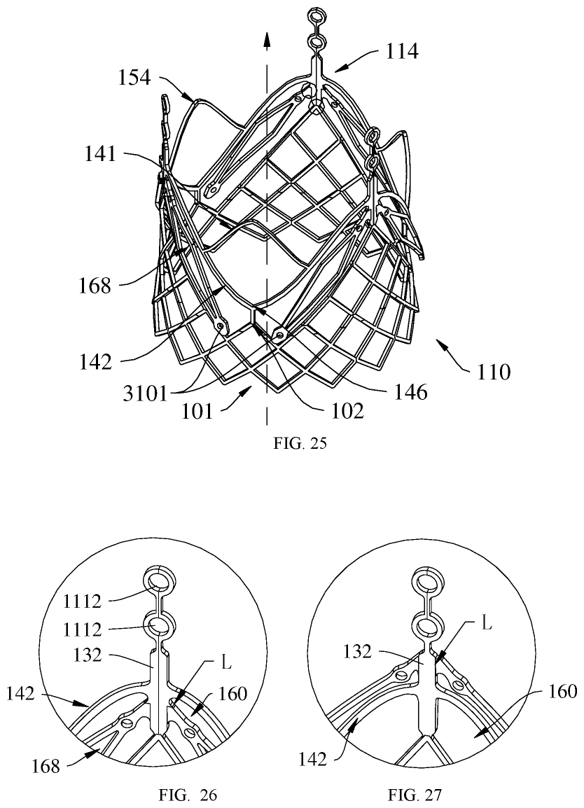

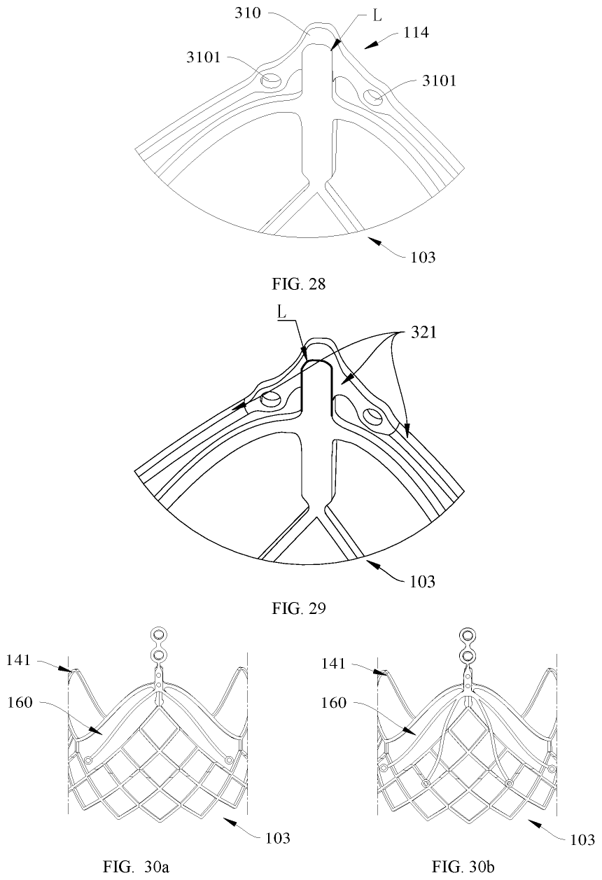

Referring to FIG. 26, the commissure region 114 is configured as a commissure post 132. In FIG. 28, each commissure post 132 extends from the outflow end 102 of the inner frame 103. Alternatively, the commissure posts 132 can extend from the interior of the inner frame 103. Specifically, the commissure post 132 can be configured as a bar, which extends along the axis of the inner frame 103 or the free end 123 of which is inclined radially inward. As shown in the figures, the commissure post 132 is configured as a solid rod. Alternatively, the commissure post 132 can be configured as a bar frame.

Referring to FIG. 26, the commissure post 132 is provided with a plurality of eyelets 1112. Alternatively, one eyelet 1112 can be provided. As shown in the figure, the plurality of eyelets 1112 on the same commissure post 132 are arranged in sequence in the axial direction of the main body of the same commissure post 132 to facilitate the processing and assembly.

The plurality of commissure posts 132 can be provided separately. As shown in FIG. 26, a second frame arm 142 is connected between adjacent commissure posts 132, and a first angled space 160 is defined between the second frame arm 142 and the outflow end 102 of the inner frame 103. In the loaded configuration, the clipping arms 120 can be located within the respective first angled spaces 160. The first angled space 160 provides motion space and accommodation space for the clipping arm 120, thereby improving the engagement of the clipping arm 120 with the inner frame. For example, in the loaded configuration, all the clipping arms 120 do not radially overlap on the inner frame 103, which improves the loaded configuration of the frame, optimizing the profile of the frame and facilitating assembly of the system as well as the treatment, wherein the small profile improves the compliance for in vivo delivery. The second frame arm 142 can extend straightly. Alternatively, as shown in the figure, the second frame arm 142 between two adjacent commissure posts 132 has a bent portion, i.e., an apex 146, and is generally V-shaped. In FIG. 25, the apex of the V-shape is fixedly connected with the edge of the inner frame 103 at the outflow end 102. Alternatively, in other embodiments, the apex of the V-shape can be free from the edge of the inner frame 103 at the outflow end 102.

The second frame arms 142 can be provided separately. Alternatively, referring to FIG. 25, the inner side of the V-shape is connected with a leg 141, and the middle portion of the leg 141 is bent to form an apex 154, which protrudes in the direction of the dashed line with an arrow as shown in the figure.

The second frame arm 142 has a V-shape, the apex 146 of which protrudes in the opposite direction to the apex 154 and is fixed with a corresponding portion of the frame 110 at the outflow end 102. The second frame arm 142 can be configured as a single rod or a deformable mesh band.

Similar to the above structure with peaks and valleys, in the present embodiment, the axial length of the inner frame 103 varies in the circumferential direction of the inner frame 103, and gradually shortens as far away from the commissure region 114. In the axial direction of the inner frame 103, the inner frame 103 has a plurality of rows of cells, including N rows of cells that respectively extend continuously in the circumferential direction adjacent the inflow end 101, and the remaining rows of cells respectively extend discontinuously in the circumferential direction, where N is 1, 2 or 3. In the circumferentially discontinuously extending rows of cells, the distance between the cells spaced from each other in the same row is larger as it is closer to the outflow end 102.

Referring to FIGS. 19 to 23b, one end of the clipping arm 120 is a fixed end 121 connected with the respective commissure region 114, and the other end is a free end 123 away from the commissure region 114. In the circumferential direction, the free ends 123 of the clipping arms 120 corresponding to adjacent two commissure regions 114 are adjacent to each other.

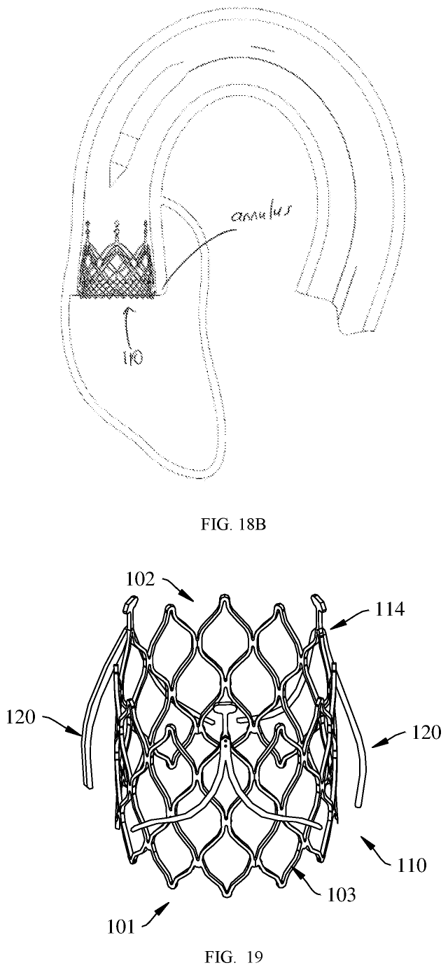

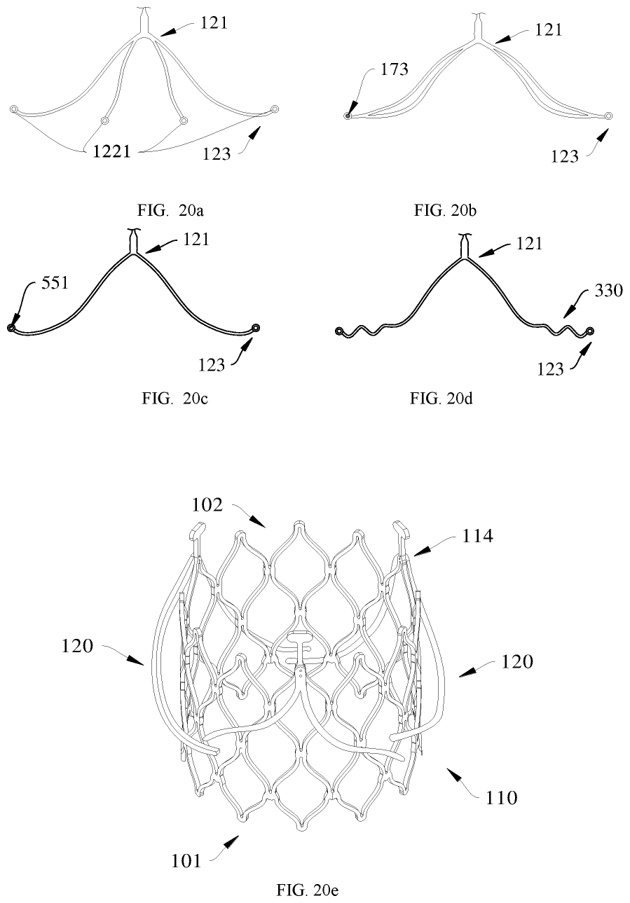

In order to better observe the position of the clipping arms 120 during the treatment, the clipping arms 120 are provided with one or more developing points 173, and at least one developing point 173 is adjacent the free end 123 of the clipping arm 120. Further, in the case of a plurality of developing points 173, at least one developing point 173 is adjacent to the free end 123 of the clipping arm 120, and at least one developing point 173 is adjacent to the fixed end 121 of the clipping arm 120. The developing point can be provided separately or share the same hole with other structure. For example, in FIG. 20c, the free end 123 of the clipping arm 120 is provided with an eyelet 551. The eyelet 551 can be used for providing the developing point 173 and also for providing a rounded structure.

In order to reduce the damage of the clipping arm 120 to the native and surrounding tissues, as shown in the figures, the free end 123 of the clipping arm 120 is configured as a rounded structure 1221. Similarly, the free end 123 of the clipping arm 120 can be provided with a protective layer. The protective layer and the rounded structure 1221 can be provided in combination.

Further, the clipping arm 120 can have a deformed structure. The deformed structure can use various forms. For example, in FIG. 25, the clipping arm 120 is provided with one or more slots 168. The slot 168 can additionally extend the length of the clipping arm 120 in the released configuration. The length of the clipping arm 120 is one of the factors that determine the position of the fixed end 121 of the clipping arm 120, and thus determines the position of the frame 110 in the physiological anatomy. Therefore, by adjusting the number, position, and size of the slots 168, the length of the clipping arm 120 can be adjusted, thereby extending the application of the prosthetic valve in the physiological anatomy. In the case where the slot 168 is small, the developing marker can be accommodated therein.

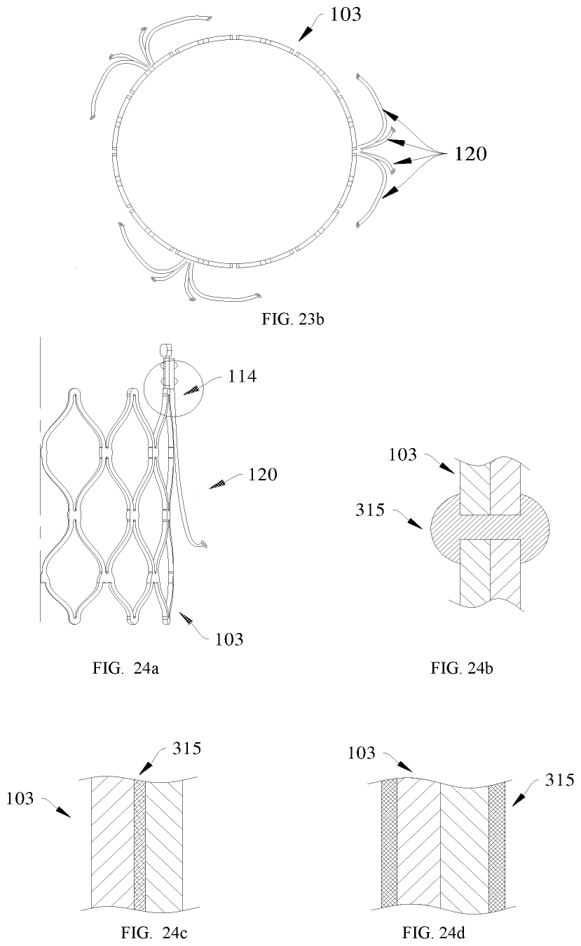

In the circumferential direction of the inner frame 103, one or more clipping arms 120 can be provided at one side of the individual commissure region 114. In the embodiment shown in FIG. 19, one single clipping arm 120 is provided at one side of the commissure region 114. The single clipping arm 120 can have a branched structure at the free end 123 thereof. Alternatively, the single clipping arm 120 can have a branched structure at the middle thereof that converges at the free end 123 as shown in FIG. 25. Alternatively, a plurality of clipping arms 120 can be provided at one side of the commissure region 114 as shown in FIG. 23b, and the clipping arms 120 can be separated from each other, or converges at the free end 123.

As shown in the figures, the clipping arm 120 is configured as a single rod. It will be conceived that the clipping arm 120 can be configured as a deformable mesh band.

In the deployed state, the angle between the clipping arm 120 and the axis of the inner frame 103 ranges from 30 to 85 degrees, where the angle is measured referring to the line connecting the two ends of the clipping arm 120.

Regarding the distribution of the clipping arms 120, the clipping arms 120 on two opposite sides of the same commissure region 114 are symmetrically distributed, and in the circumferential direction of the inner frame 103, the clipping arms 120 between the adjacent commissure regions 114 are symmetrically distributed.

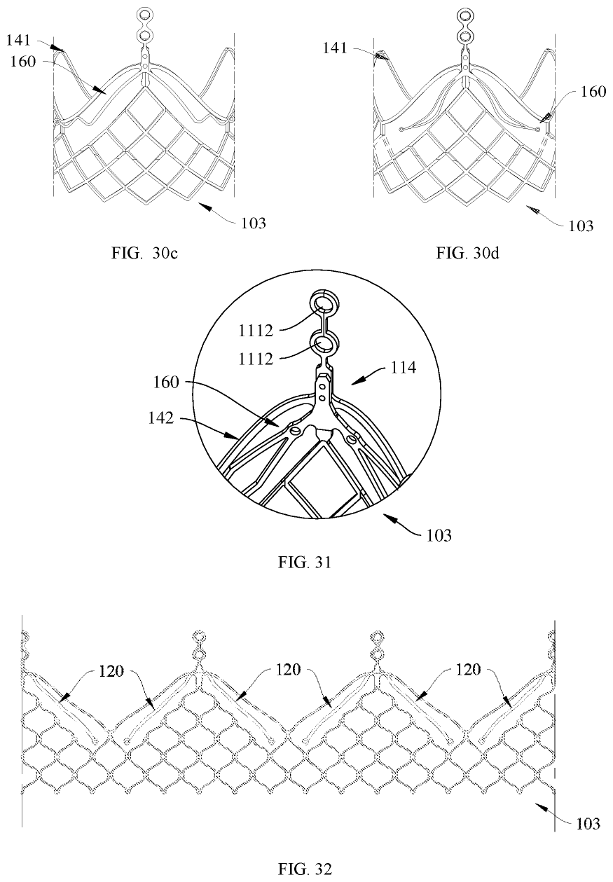

FIGS. 30a to 31 show the engagement between the clipping arms 120 and the inner frame, wherein the clipping arms 120 are all connected to the inner frame by riveting. As shown in FIGS. 30a to 30d, the clipping arm is connected with the outer peripheral surface of the inner frame, while in FIG. 31, the clipping arm is connected with the inner peripheral surface of the inner frame. It can be conceived that the clipping arms shown in FIGS. 30a to 30d can be connected with the inner peripheral surface of the inner frame.

The fixed ends 121 of the clipping arms 120 can be connected with the inner frame 103 separately or in combination. In the embodiment shown in FIG. 26 to 29, the fixed ends 121 of the clipping arms 120 in the same group converge to a connecting portion 310 and are fixed to the inner frame 103 by the connecting portion 310. The connecting portion 310 and the clipping arms 120 joined to the connecting portion 310 can be formed in one piece, for example, by cutting or knitting. Further, in the circumferential direction of the inner frame 103, the clipping arms 120 in the same group are distributed on two sides of the connecting portion 310. In practice, the inner frame 103 is provided with at least two commissure regions 114 at intervals in the circumferential direction, and the connecting portions 310 are respectively fixed to the commissure regions 114 on the inner frame 103 by welding or by connecting members 315.

The connecting portion 310 can connect the clipping arms 120 and the inner frame 103 better while adapting different expansion characteristics of the two. Further, the engagement between the connecting portion 310 and the commissure region 114 can be various. For example, referring to FIG. 24a, the connecting portion 310 is overlapped on the outer side of the commissure region 114 in the radial direction of the inner frame 103. Referring to FIG. 31, the connecting portion 310 is overlapped on the inner side of the commissure region 114 in the radial direction of the inner frame 103. Referring to FIG. 26, in the circumferential direction of the frame 110, the connecting portion 310 is located on one circumferential side of the commissure region 114, that is, the connecting portion 310 does not radially overlap on the commissure region 114. Referring to FIGS. 28 and 29, the connecting portion 310 covers the top of the commissure region 114. Compared with FIG. 24a and FIG. 31, the junction in FIG. 26, FIG. 28 and FIG. 29 is more invisible and cannot be clearly shown in the figures, and thus is represented by a thick line L. The above-mentioned various configurations have different advantages in terms of assembly difficulty and volume in the loaded configuration.

In the case where the connecting member is used for fixing, the specific implementation of the connecting member 315 can refer to FIGS. 24b to 24d. In the figures, the connecting member 315 is configured as a fixing member passing through the connecting portion 310 and the commissure region 114. Specifically, with reference to FIG. 24b, the connecting member 315 can be configured as a screw, a rivet, a binding wire, or the like. Alternatively, as shown in FIG. 24c, the connecting member 315 can be configured as a sandwiched adhesive lay. Alternatively, as shown in FIG. 24d, the connecting member 315 can be configured as a covering structure.

As shown in FIG. 28, the connecting portions 310 corresponding to the clipping arms 120 in the same group are formed in one piece. Referring to FIG. 29, the connecting portions 310 corresponding to the clipping arms 120 in the same group can be separated and adjacent to each other. Further, the separated structure includes a plurality of units 321, which are separated and respectively connected to the commissure region 114 of the inner frame 103, or the plurality of units 321 can be fixed to each other, with at least one unit 321 connected to the commissure region 114 of the inner frame 103.

Similarly to the developing arrangement in the clipping arm 120, the connecting portion 310 can also be provided with a developing hole(s) 3101 for mounting the developing element(s), in order to provide a more clear observation.

The clipping arm 120 can use various forms. FIGS. 20a to 20d, 22a to 22d, and 23a to 23b shows different perspectives of the clipping arms with different arrangements.

FIGS. 20a to 20d show front view of FIG. 19, which can be considered as showing the clipping arm projected on the paper in the front view; in the case where the clipping arm, when projected on the paper, has no configuration as shown in the figures, the shown clipping arm can be considered as the configuration being flattened.

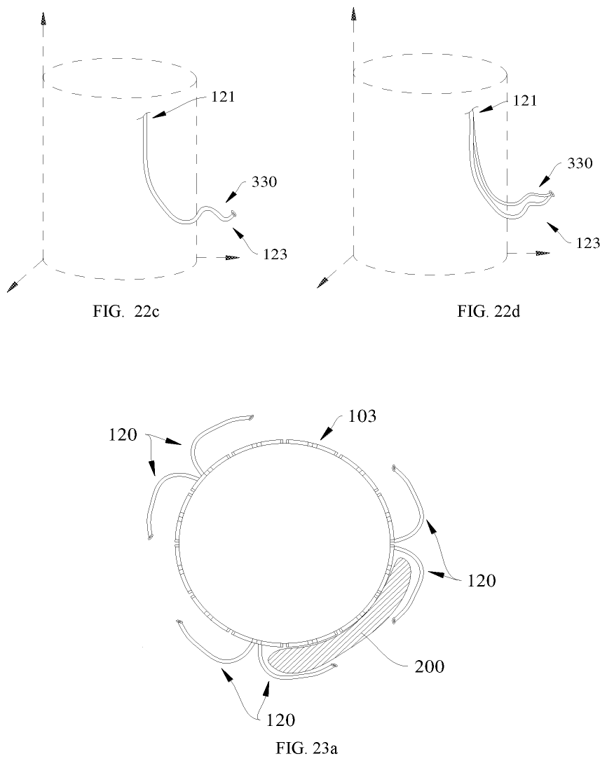

FIGS. 22a to 22d show the released configuration of the clipping arm in a cylindrical coordinate, wherein the dotted line shows the cylindrical coordinate. In order to better show the three-dimensional configuration of the clipping arm in the two-dimensional figures, the cylinder of the cylindrical coordinate in the figure is depicted referring to the profile of the frame, so these figures can be approximately understood as showing the spatial relationship between the frame and the clipping arm.

FIGS. 23a to 23b show top views of FIG. 19, and can be understood as showing the clipping arm projected on the paper in the top view; in the case where the clipping arm, when projected on the paper, has no configuration as shown in the figures, the shown clipping arm can be considered as the configuration being flattened.

Referring to FIG. 20d, FIG. 22c, and FIG. 22d, the clipping arm 120 has a wave structure 330 adjacent to the free end 123. In the figures, the wave structure 330 mainly undulates in the axial direction of the frame 110. It can be conceived that the clipping arm 120 can have undulations in multiple directions in space. Referring to FIGS. 23a and 23b, the clipping arm 120 has a radially undulating structure as viewed in the axis of the inner frame 103. The undulations in multiple directions can be provided separately or overlapped with each other to form a complex three-dimensional configuration.

The clipping arms 120 can be divided into a plurality of groups, depending on the position of the fixed ends 121. Each group of clipping arms 120 can include one or more pairs of clipping arms 120. In the embodiment shown in FIG. 23b, the same group includes multiple pairs of clipping arms 120. In the circumferential direction of the inner frame 103, the clipping arms 120 in the same pair are respectively located on two sides of the connecting portion 310, and the clipping arms 120 in different pairs have different lengths after being released.

In another aspect, in the circumferential direction of the inner frame 103, the clipping arms 120 in the same group are divided into pairs of clipping arms 120, and the clipping arms 120 in the same pair are respectively located on two sides of the connecting portion 310. In the released configuration, the clipping arms 120 on the same side of the connecting portion 310 while in different pairs have different extensions.

The different configurations described above represent the three-dimensional configurations of the clipping arm 120 in the released configuration. Further, referring to FIG. 23 a, in the released configuration, the free ends 123 of the clipping arms 120 in the same group are located at the same radial position relative to the inner frame 103. Referring to FIG. 23b, in the released configuration, the free ends 123 of the clipping arms 120 in the same group are offset from each in the radial direction relative to the inner frame 103. However, the free ends 123 of all the clipping arms 120 in the afore-mentioned two cases are both located between the two ends of the inner frame 103 in the axial direction of the inner frame 103 in the released configuration, wherein the two ends of the inner frame 103 in the present embodiment refer to the inflow end 101 and the outflow end 102 of the inner frame 103, so as to prevent the clipping arms 120 from affecting the release and positioning of the frame 110.

The plurality of clipping arms 120 can use the same configuration as describe above. Alternatively, the plurality of clipping arms 120 can use different configurations in one embodiment as shown in FIG. 20e. Specifically, two adjacent clipping arms 120 in different groups have different lengths. Further, the clipping arms 120 in the same group can be different. For example, the two clipping arms 120 in the same group can have different lengths. Besides the difference in the extension length of the clipping arms 120, the free ends of adjacent two clipping arms in different groups can be offset from each other in the circumferential direction of the inner frame. As shown in the figure, the clipping arm 120 has a bent portion adjacent to the free end thereof so as to change the extension path thereof. In the deployed state, the bent portion of one of the adjacent clipping arms surrounds the free end of the other in half, which further improves the positioning of the clipping arms 120 on the native leaflet.

Referring to FIGS. 32 to 37j, the present application discloses a frame 110 for a prosthetic heart valve device, including:
an inner frame 103 having a meshed cylindrical structure, which has relative compressed configuration and expanded configuration depending on the radial deformation, and a support device (e.g., a balloon) for driving the inner frame 103 to transform into the expanded configuration can be placed within the inner frame 103;
a connecting ring 340 fixed with the outflow end 102 of the inner frame 103 and provided with a plurality of connecting regions 341 at intervals; and
a plurality of groups of clipping arms 120 which are located at an outer periphery of the inner frame 103 and spaced in the circumferential direction of the frame 110, each clipping arm 120 having opposite fixed end 121 and free end 123, and the fixed ends 121 of clipping arms 120 in the same group are located at the same connecting region 341.

The clipping arm 120 is made of memory material and has configurations of:
a loaded configuration, in which the inner frame 103 assumes the compressed configuration, and the clipping arms 120 contact the inner frame 103; and
a released configuration, in which the inner frame 103 assumes the expanded configuration, the free end 123 of each clipping arm 120 expands radially outward, with a space defined between the free end 123 of each clipping arm 120 and the inner frame 103 to allow entry of the native leaflet 201

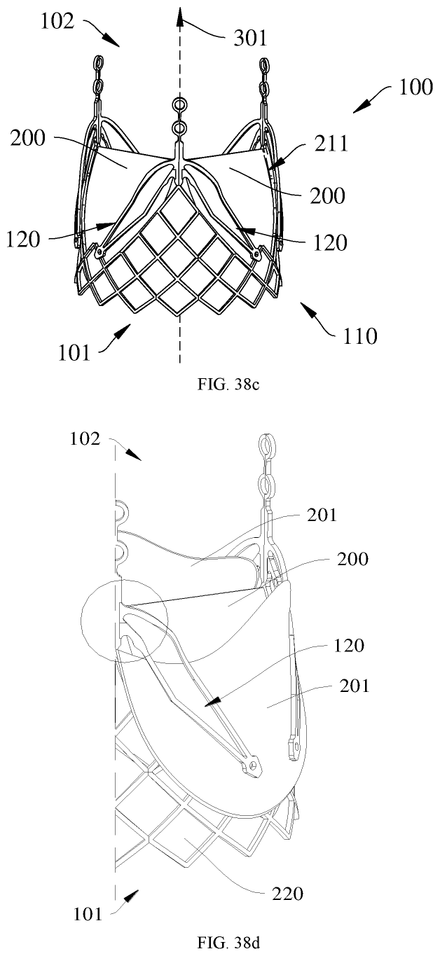

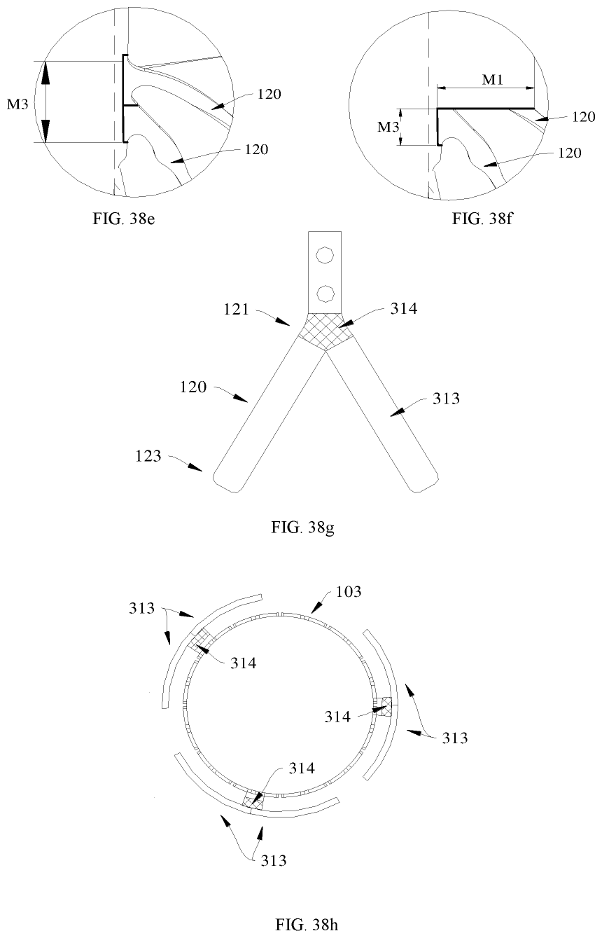

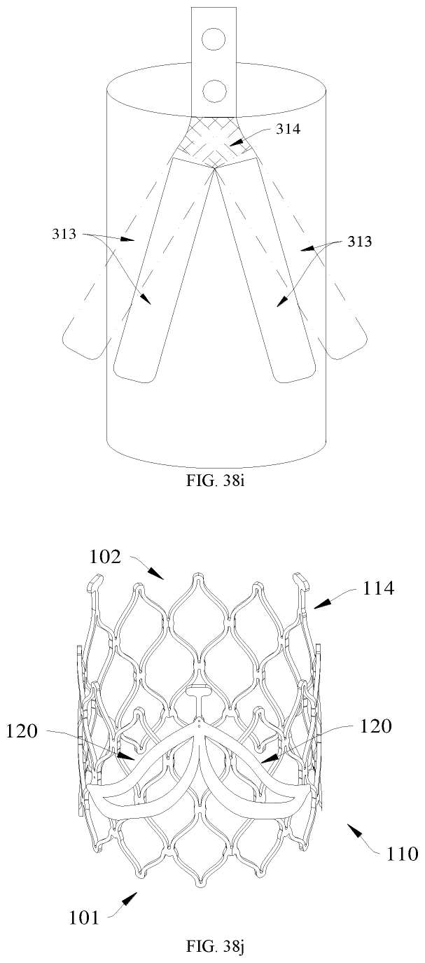

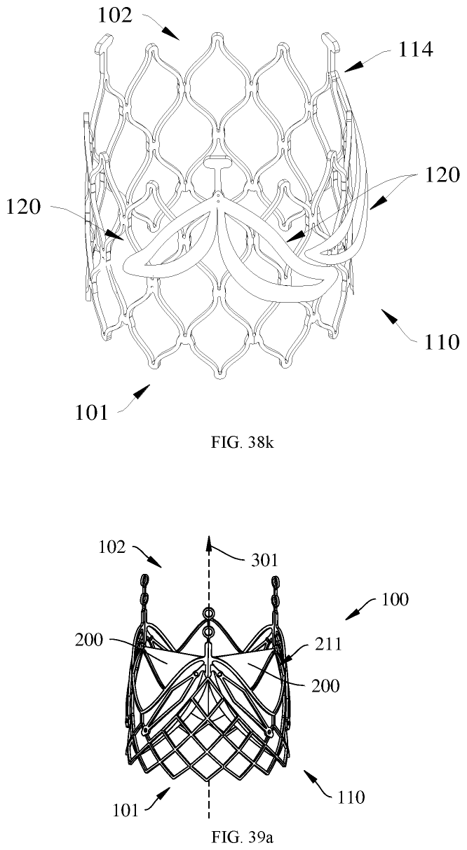

In this embodiment, the plurality of groups of the clipping arms 120 are connected by the connecting ring 340, so that the clipping arms 120 and the inner frame 103 can be separately provided with more flexibility. In general, the free ends 123 of at least two clipping arms 120 in the same group tend to extend away from each other, and the free ends 123 of at least two clipping arms 120 in adjacent two groups tend to extend close to each other. The clipping arms 120 forms a deformable deployed structure on the outer periphery of the inner frame 103. The clipping arm 120 can be connected with the inner frame by the connecting portion 310. Specifically, in one embodiment, the connecting portion 310 can be overlapped on the outer side of the commissure region 114 in the radial direction of the inner frame 103. Alternatively, in another embodiment, the connecting portion 310 can be overlapped on the inner side of the commissure region 114 in the radial direction of the inner frame 103. Alternatively, in a further embodiment, the connecting portion 310 can be connected at one circumferential side of the commissure region 114 in the circumferential direction of the frame 110, that is, the connecting portion 310 does not radially overlap on the commissure region 114. In FIGS. 38e and 38f, the junction is more invisible and cannot be clearly shown in the figures, and thus is represented in a bold in the figures. The above-mentioned various configurations have different advantages in terms of assembly difficulty and volume in the loaded configuration.

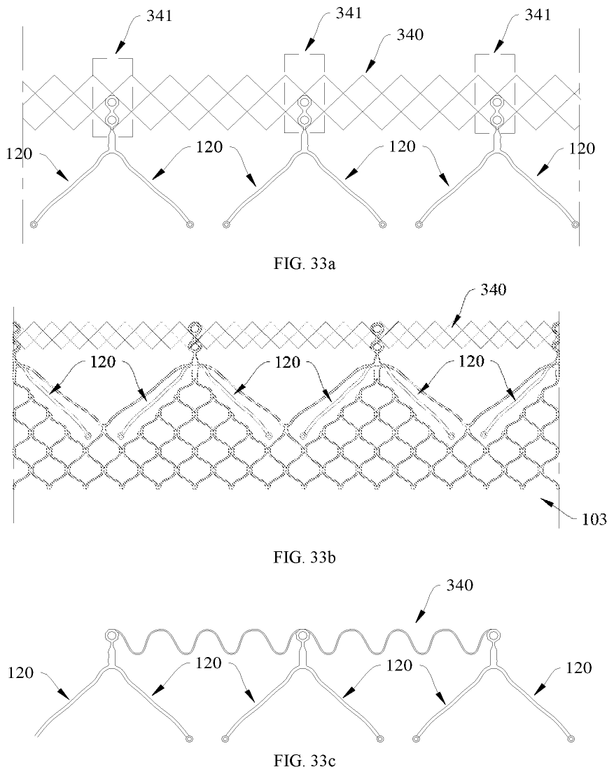

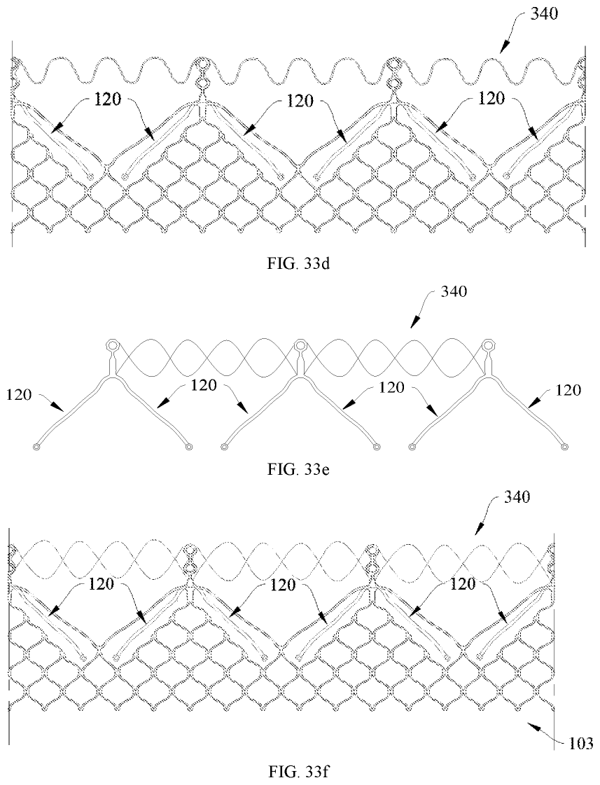

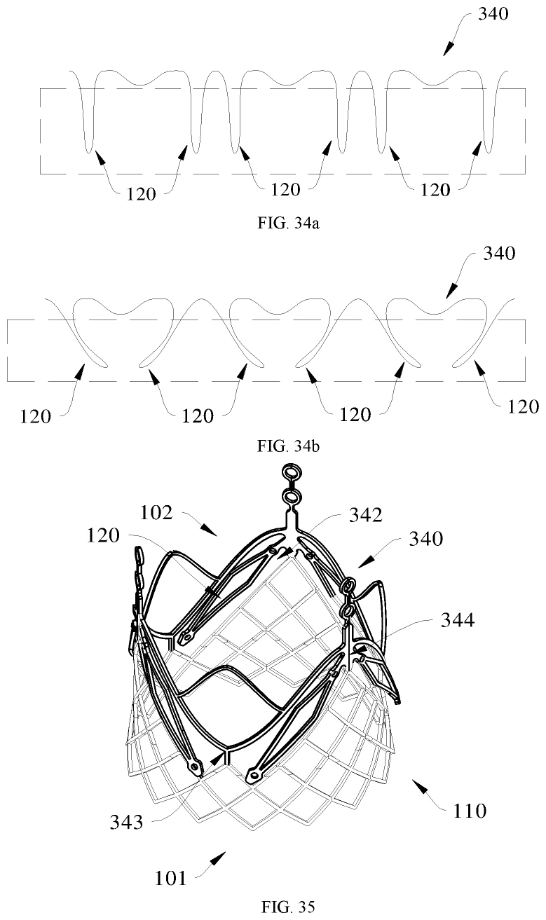

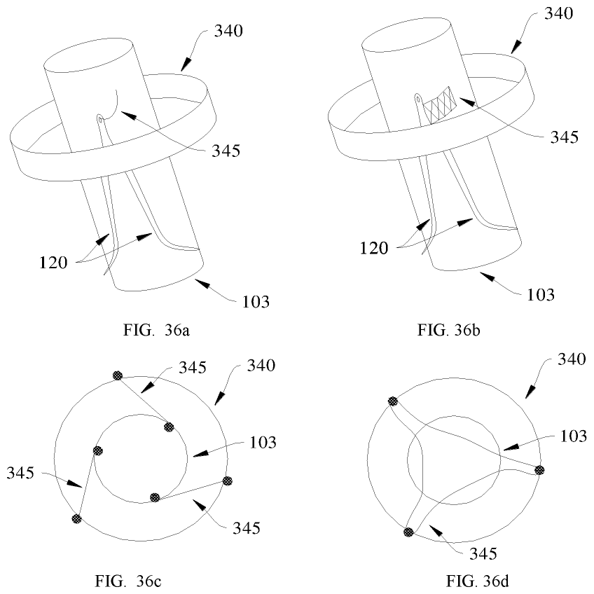

Referring to FIG. 33a, the fixed ends 121 of the clipping arms 120 in different groups are located at different connecting regions 341. Referring to FIG. 36a, the connecting ring 340 surrounds and is connected with the outer periphery of the inner frame 103. Alternatively, referring to FIG. 35, the connecting ring 340 is connected with one axial end of the inner frame 103. Further, the connecting ring 340 is configured as a radially deformable structure. In practice, the connecting ring 340 can be configured as a mesh band. Referring to FIGS. 34a and 34a, the connecting ring 340 is configured as a single-strand strip, which extends along the circumferential direction of the inner frame 103, and has a wave structure 330 undulating in the axial direction of the inner frame 103.

In the released configuration, an independent space is defined between the connecting ring 340 and the clipping arms 120, so that the connecting ring 340 can be more flexibly engaged with the inner frame 103. Referring to FIG. 35, the inflow end 101 of the coupling ring 340 is connected with the outflow end 102 of the inner frame 103, which allows the connecting ring 340 and the inner frame 103 to be offset from each other in the axial direction. Further, a second angled space 342 is defined between the inflow end 101 of the connecting ring 340 and the outflow end 102 of the inner frame 103, and the clipping arms 120 are located within the respective second angled space 342 in the loaded configuration. Specifically, the connecting ring 340 is connected with the inner frame 103 at a first position 343 and/or a second position 344. Different connection positions and numbers affect the mechanical performance of the connecting ring 340, thereby affecting the movement of the clipping arms 120. The clipping arm 120 is connected with the connecting ring 340 at the second position 344. The first position 343 and the second position 344 are offset from each other in the circumferential direction of the inner frame 103, and the offset angle is 360/2n, where n is the number of leaflets 200 configured to be loaded in the frame 110. As shown in the figure, the frame 110 shown is used for a tricuspid valve, so the first position 343 and the second position 344 are offset by an angle of 60 degrees.

Independent from the above configuration, the coupling ring 340 and the inner frame 103 do not overlap each other, so that they are allowed to be compressed into a desired volume, which facilitates the treatment.

The axial offset and the radial offset between the connecting ring 340 and the inner frame 103 can be achieved separately and independently, or in combination as shown in the figures.

Regarding the connection between the groups of clipping arms 120 and the inner frame 103, see FIG. 35, the fixed ends 121 of the clipping arms 120 in the same group converge to the connecting ring 340 adjacent the outflow end 102 of the inner frame 103. As shown in FIG. 35, the connecting ring 340 is rigidly fixed with the commissure region 114. As shown in FIGS. 36a and 36b, a motion space is defined between the coupling ring 340 and the commissure region 114. In order to allow the clipping arms 120 to restrict the inner frame 103, it can be understood that axial restraint should be provided between the connecting ring 340 and the commissure region 114. Specifically, the connecting ring 340 and the commissure region 114 can be movably engaged with each other in the circumferential direction of the frame 110, with axial restraint therebetween; or the connecting ring 340 and the commissure region 114 can be movably engaged with each other in the radial direction of the frame 110, with axial restraint therebetween.

Besides the rigid connection, as shown in FIGS. 36a-36d, the connecting ring 340 and the commissure region 114 can be connected by a flexible member 345. Specifically, the flexible member 345 movably passes through the inner frame 103, and changes the allowable axial movement between the inner frame 103 and the clipping arm 120 when the configuration of the inner frame 103 is transformed. The flexible member 345 can use a binding wire made of a polymer material, a deformable member made of a metal or plastic material, or the like. Specifically, the flexible member 345 can be a single wire as shown in FIG. 36a or a mesh band as shown in FIG. 36b.

Besides the above-mentioned connection methods for the connecting ring 340 and the clipping arms 120, as shown in FIGS. 34a and 34b, the connecting ring 340 and the clipping arms 120 can be formed in one piece. Further, the connecting ring 340 and the clipping arms 120 are formed by winding a wire(s). In practice, the wire is configured as a single wire without a break. The wire can be made of an alloy material having a memory effect.

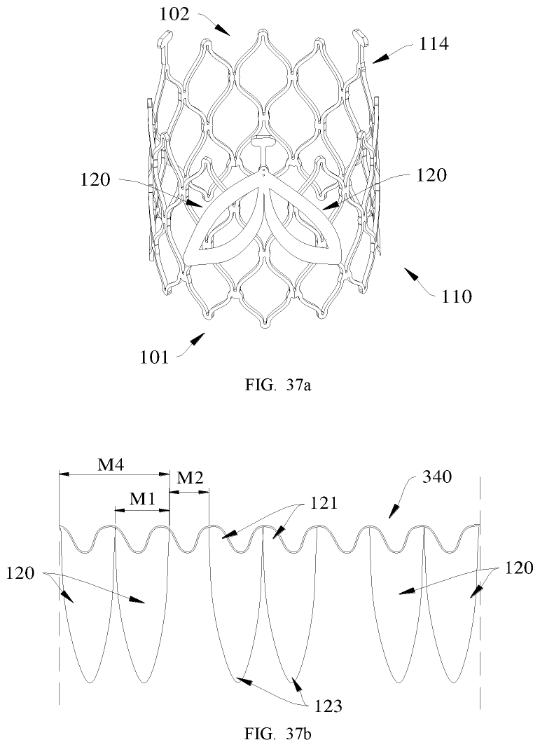

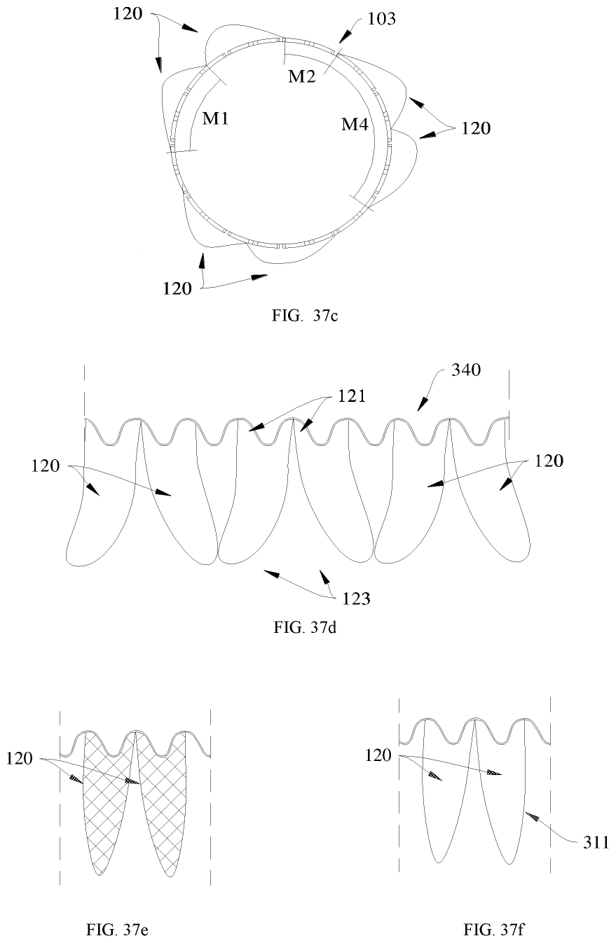

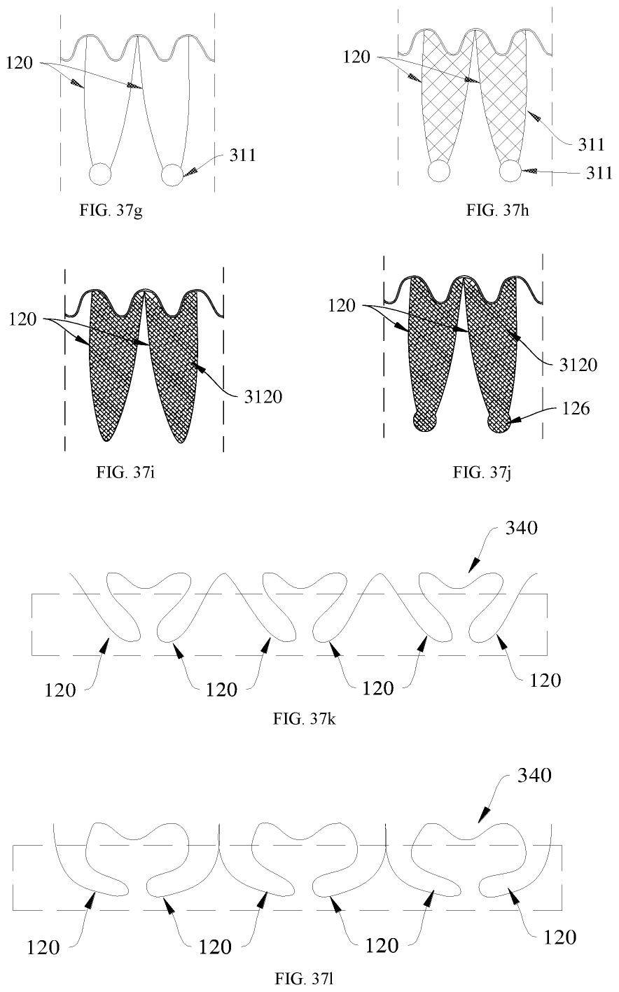

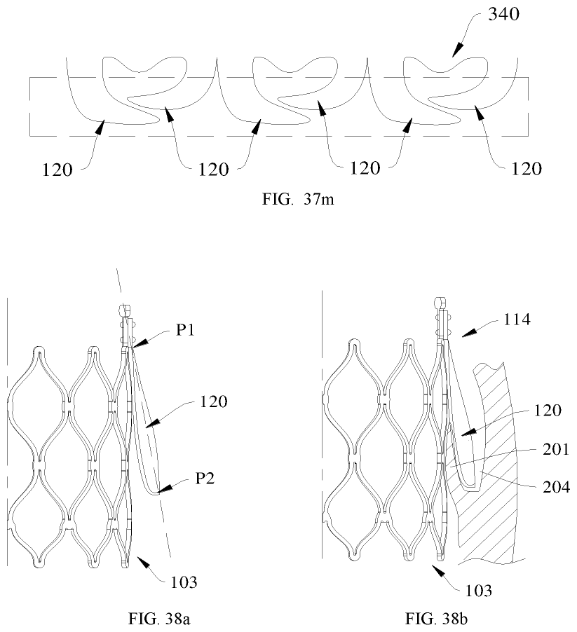

Referring to FIG. 37a, the present application further discloses a frame 110 for a prosthetic heart valve device, including:
an inner frame 103 having a meshed cylindrical structure, which has relative compressed configuration and expanded configuration depending on the radial deformation, and a support device for driving the inner frame 103 to transform into the expanded configuration can be placed within the inner frame 103; and
clipping arms 120, each of which has opposite fixed end 121 and free end 123, wherein the fixed end 121 is connected with the inner frame 103, and the clipping arm 120 satisfies at least one of the following conditions with respect to the axis of the inner frame 103:
the circumferential distribution region M1 of the fixed end 121 has a central angle greater than 15 degrees with respect to the axis; and
the length of the axial distribution region M3 of the fixed end 121 is greater than 5 mm;

The clipping arm 120 is made of memory material and has configurations of:
a loaded configuration, in which the inner frame 103 assumes the compressed configuration, and the clipping arms 120 contact the inner frame 103; and
a released configuration, in which the inner frame 103 assumes the expanded configuration, the free end 123 of each clipping arm 120 expands radially outward, with a space defined between the free end 123 of each clipping arm 120 and the inner frame 103 to allow entry of the native leaflet 201.

FIG. 37a shows a group of reinforced clipping arms 120. In this embodiment, on the one hand, the frame is self-expanded by means of a support device (such as a balloon), on the other hand, the separate clipping arms 120 are reinforced by optimizing the shape or size thereof, thereby improving the positioning effect of the clipping arms 120 on the native leaflet 201. Compared with the clipping arm 120 configured as a single rod or the like, the reinforced clipping arm 120 according to this embodiment has a more stable positioning effect.

Specifically, the circumferential distribution region M1 and the axial distribution region M3 of the fixed end 121 of the individual clipping arm 120 are improved, to ensure the connection strength of the single clipping arm 120 with the inner frame 103 as well as the spatial shaping performance thereof.

Regarding the connection between the clipping arms 120 and the inner frame 103, as shown in FIGS. 37b to 37m, the clipping arms 120 are connected with a separate connecting ring, and then connected to the inner frame 103 by the connecting ring to form a frame with separate pieces. Alternatively, as shown in FIGS. 28a to 38f, the clipping arms 120 are directly connected to the inner frame 103 to form a frame with a one piece. In FIGS. 37k, 37l, and 37m, the connecting ring and the clipping arms 120 are formed by winding a wire, but the clipping arms 120 in FIGS. 37k, 37l, and 37m have different shapes. As shown in FIG. 37l, the clipping arm 120 extends approximately in the axial direction of the inner frame 103, and then turns to extending approximately in the circumferential direction of the inner frame 103. As shown in FIG. 37m, the clipping arms 120 in the same group can be asymmetrically arranged.

Referring to FIGS. 37a to 38f, the clipping arms 120 are arranged in groups, and the fixed ends 121 of the clipping arms 120 in the same group are adjacent to each other. Comparing FIGS. 37b and 37d, it can be seen that the free ends 123 of the clipping arms 120 can be moved away from or closer to each other to achieve different positioning effects. In order to avoid interference between the clipping arms 120 in the circumferential direction, the clipping arm 120 is sized so that the central angle of the circumferential distribution region M4 of the fixed ends 121 of the clipping arms 120 in each group with respect to the axis is equal to or less than 360/n, where n is the number of leaflets 200 configured to be loaded in the frame 110. As shown in the figures, the frame 110 is used for a tricuspid valve, and thus the central angle of the circumferential distribution region M3 of the fixed ends 121 of the clipping arms 120 in each group with respect to the axis is less than or equal to 120 degrees.

The central angle of the circumferential distribution region M1 of the fixed end 121 of the clipping arm 120 with respect to the axis is equal to or less than 360/2n, where n is the number of leaflets 200 configured to be loaded in the frame 110. Similarly, as shown in the figures, the central angle of the circumferential distribution region M1 of the fixed end 121 of the clipping arm 120 with respect to the axis is smaller than or equal to 60 degrees.

The above-described parameters can avoid a reduced freedom of motion of the clipping arm 120 caused by the increased size, thereby ensuring the positioning effect.

Referring to FIG. 37b, FIG. 37c and FIG. 38f, it can be seen that the circumferential distribution region M1 shown in the figures represents the projection dimension of the fixed end 121 of the clipping arm 120 in the circumferential direction of the inner frame 103, and similarly, the axial distribution region M3 represents the projection dimension of the fixed end 121 of the clipping arm 120 in the axial direction of the inner frame 103. Referring to FIGS. 38e and 28f, depending on the different configurations of the fixed end 121, the circumferential distribution region M1 and the axial distribution region M3 are adjusted correspondingly, and the specific junction (shown by a thick solid line in the figures) between the clipping arm 120 and the inner frame 103 is also changed correspondingly.

In general, when projected onto the peripheral surface of the inner frame 103, the clipping arm 120 assumes a sheet-like structure having a certain area. Referring to FIG. 37a, FIG. 37d and FIG. 38a, the clipping arm 120 generally has a curved extension from the fixed end 121 to the free end 123, and two clipping arms 120 in adjacent groups cooperate each other to conform to the anatomic structure of the valvular sinus 204. Specifically, and for example, within two clipping arms 120 in adjacent two groups, the overall structure gradually converges from the outflow end 102 to the inflow end 101, that is, the span between the two clipping arms 120 in the circumferential direction of the inner frame 103 gradually decreases until the free ends 123 thereof are close to each other. With regard to the specific extension path, the clipping arm 120 can extend uniformly in the circumferential and axial directions of the inner frame 103 as shown in FIG. 37a, or first extends approximately in the circumferential direction of the inner frame 103 and then turns to extending approximately in the axial direction of the inner frame 103 as shown in FIG. 38d, or refer to FIG. 38j.

At least a spacing region M2 is defined between the fixed ends 121 of the two clipping arms 120 in adjacent groups in the circumferential direction of the inner frame 103, wherein the spacing region M2 has a center angle relative to the axis greater than 30 degrees, for example, 60 to 120 degrees. The spacing region M2 can reduce interference between clipping arms 120 in adjacent groups and low the risk of simultaneous failure.

With regard to the specific structure of the clipping arm 120, each of the clipping arms 120 has a multi-bar structure from the fixed end 121 to the free end 123. The multi-bar structure is configured so that there are at least two bars of the clipping arm 120 at any portion in any direction which can be one or more of the axial direction, the radial direction, and the circumferential direction of the frame 110. Alternatively, referring to FIG. 37e, each of the clipping arms 120 is configured as a mesh band consisting of bars, and there are at least two bars at any position in the extension direction of the clipping arm 120. In the case where the clipping arm 120 is configured as a single bar, the strength there will be inevitably decreased, affecting the overall strength and positioning effect. The mesh band is generally configured as a sheet-like structure, where the clipping arm 120 extends in a single layer or in double layers from the fixed end 121 to the free end 123. Further, a solid sheet-like structure, i.e., a relatively closed sheet-like structure in space, can be formed by filling the hollowed-out regions of the mesh band of the clipping arm 120, and the specific filter can be a polymer material or a metal material. In the case where the filter chooses the same material as the bars of the clipping arm 120, the clipping arm 120 is generally formed as a leaf-shaped metal sheet.

However, the clipping arm, which is generally formed as a metal sheet, results in problems in switching between the loaded configuration and the released configuration. In the embodiment shown with reference to FIG. 38g, the portion of the clipping arm 120 adjacent to the free end 123 is configured as an undeformable rigid portion 313. The undeformable rigid portion 313 should be understood as a portion which is designed to be undeformable, rather than being a rigid body in strict mechanical meaning. Referring to FIG. 38i, the rigid portion 313 maintains the same or similar shape and form both in the loaded configuration and the released configuration. In terms of mechanical properties, the deformation resistance of the rigid portion 313 is significantly higher than that of other portions of the clipping arm 120, particularly the flexible portion 314 mentioned below. Specifically, the rigid portion 313 can be implemented by a specific rigid material, or by a specific rigid structure. As shown in the figures, the rigid portion 313 is configured as a solid sheet-like structure.

The rigid portion 313 is provided so that the self-deformation of the clipping arm 120 is concentrated on the fixed end 121, which can be implemented by weakening the mechanical properties of the fixed end 121. Alternatively, referring to one embodiment, the fixed end 121 of the clipping arm 120 is configured as a deformable flexible portion 314. Alternatively, referring to another embodiment, the clipping arms 120 in the same group are connected to each other through a deformable flexible portion 314. The difference between the two embodiments is that the flexible portion 314 is provided by the clipping arm 120 or independent from the clipping arm 120. The flexible portion 314 can be implemented by a flexible material, or can be implemented by a flexible structure. For example, as shown in the figures, the flexible portion 314 is configured as a mesh band.

The rigid portion 313 and the flexible portion 314 fit with each other depending on the respective distribution proportions thereof on the clipping arm. In principle, the rigid portion is at least 50% of the total length of the clipping arm in the extension direction of the clipping arm. Further, referring to FIG. 38g, the proportion can be adjusted to 65% or more.

The flexible portion 314 mainly functions to realize the deformation of the rigid portion 313 with respect to the inner frame, that is, switching between the loaded configuration and the released configuration. Referring to FIG. 38i, in the loaded configuration, the clipping arms in the same group are close to each other and surround the outer periphery of the inner frame. Referring to FIG. 38h, the clipping arms do not overlap each other in the radial direction of the inner frame, thereby improving the overall profile of the frame in the loaded configuration. From another perspective, the sum of the projection lengths of the clipping arms in the axial direction of the frame is less than or equal to the circumferential length of the frame. In the illustrated embodiment, the projection lengths of the clipping arms in the axial direction of the frame are the same.

The plurality of clipping arms 120 can use the same configuration as describe above. Alternatively, the plurality of clipping arms 120 can use different configurations in one embodiment as shown in FIG. 20e. Specifically, two adjacent clipping arms 120 in different groups have different lengths. Further, the clipping arms 120 in the same group can be different. For example, the two clipping arms 120 in the same group can have different lengths. Besides the difference in the extension length of the clipping arms 120, the free ends of adjacent two clipping arms in different groups can be offset from each other in the circumferential direction of the inner frame. As shown in the figure, the clipping arm 120 has a bent portion adjacent to the free end thereof so as to change the extension path thereof. In the deployed state, the bent portion of one of the adjacent clipping arms surrounds the free end of the other in half, which further improves the positioning of the clipping arms 120 on the native leaflet. This asymmetric arrangement can also be applied to the embodiment shown in FIG. 37m.

An additional part can be further provided on the clipping arm 120. With reference to FIGS. 37f and 37g, each clipping arm 120 is provided with an enlarged positioning structure 311. The positioning structure 311 can facilitate the positioning of the clipping arm 120 on the native valve leaflet 201 and prevent the clipping arm 120 from falling off the valvular sinus 204. Specifically, as shown in FIG. 37g, the positioning structure 311 is provided at the free end 123 of the respective clipping arm 120 and is enlarged by extension of the material of the clipping arm 120 itself. Further, the positioning structure 311 is configured as an enlarged sphere. As shown in FIG. 37f, the positioning structure 311 is configured as a thickened region on the clipping arm 120, specifically at the edge. The positioning structure 311 is provided at a side edge of the clipping arm 120 extending from the fixed end 121 to the free end 123 thereof. In other words, the positioning structure can be provided at the portion of the clipping arm 120 which is configured to contact the bottom or edge of the sinus of the native leaflet 201. The specific form of the positioning structure 311 can be a positioning sphere, a positioning flange, a positioning bump, or the like. Alternatively, referring to FIG. 37h, different positioning structures 311 can be provided on the same clipping arm 120, which cooperate with each other.

In addition to the positioning structure 311, referring to FIG. 37i and FIG. 37j, each of the clipping arms 120 can be covered with a sleeve 3120, which can use a braided structure or be formed in one piece. The sleeve 3120 can provide more functions for the clipping arm 120. For example, the sleeve 3120 can be made of a biocompatible polymer material. In this embodiment, the sleeve 3120 enables the surrounding tissue to be attached and fixed to the clipping arm 120, thereby further improving the positioning effect. For another example, the sleeve 3120 can be provided with a drug-loading space. The drug-loading space can be a separate space, or can be a gap(s) in the sleeve 3120 of the braided structure as mentioned above. In this embodiment, the sleeve 3120 can facilitate the treatment by applying drug. Referring to FIG. 37j, the sleeve 3120 and the positioning structure 311 as described above can be provided on the same clipping arm 120, which cooperate with each other.

Similar to the other clipping arms 120, referring to FIG. 38a, the line connecting the center P1 of the fixed end 121 and the center P2 of the free end 123 of each clipping arm 120 is defined as the clipping path which is not coplanar with the axis of the inner frame 110. When fitting the native valve leaflet 201, as shown in FIG. 38b, the increased size of the clipping arm 120 can better fit the anatomic structure of the valvular sinus 204, thereby achieving a better positioning effect.

Referring to FIGS. 38c to 38f, the present application discloses a prosthetic heart valve device with reinforced clipping arms 120, including a frame 110 as described above and valve leaflets 200, wherein the leaflets 200 are connected with the frame 110 and configured to be located within the blood flow channel 301. The leaflets 200 cooperate with each other for opening or closing the blood flow channel 301.

The inner and/or outer sides of the inner frame 103 can be further provided with a covering film 220. The two leaflets 200 adjacent in the circumferential direction are connected to a joining region 211 on the inner frame 103, and the commissure region 114 corresponds to a corresponding joining region 211 in the circumferential direction of the inner frame 103.

The specific operation of the prosthetic heart valve device 100 can be concluded referring to the above description of the frame and would not be described here. The delivery process of the prosthetic heart valve device 100 will be described in detail below with respect to the delivery system.

The present application further discloses a prosthetic heart valve device 100, including a frame 110 and leaflets 200, wherein the frame 110 is any of the frames 110 as described above, the leaflets 200 are connected to the frame 110 and configured to be located in the blood flow channel 301. The leaflets 200 cooperate with each other for opening or closing the blood flow channel 301.

The inner and/or outer sides of the inner frame 103 can be further provided with a covering film 220. The two leaflets 200 adjacent in the circumferential direction are connected to the joining regions 211 of the inner frame 103, and the connecting regions 114 correspond to the corresponding joining regions 211 in the circumferential direction of the inner frame 103.

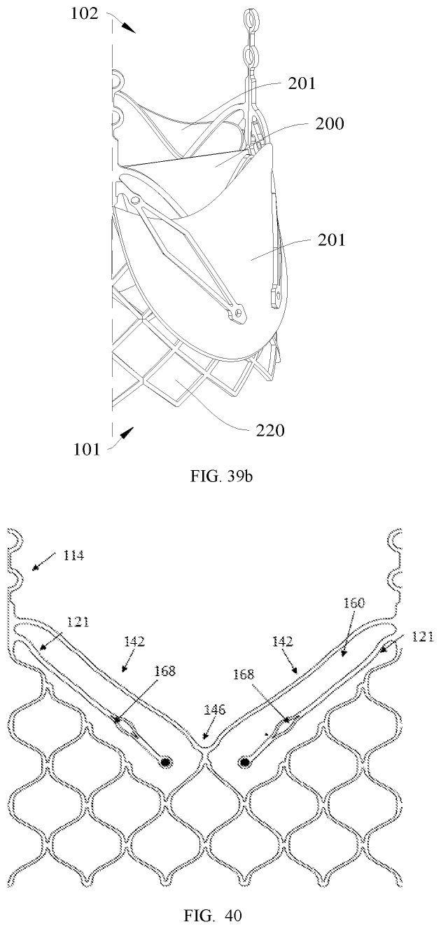

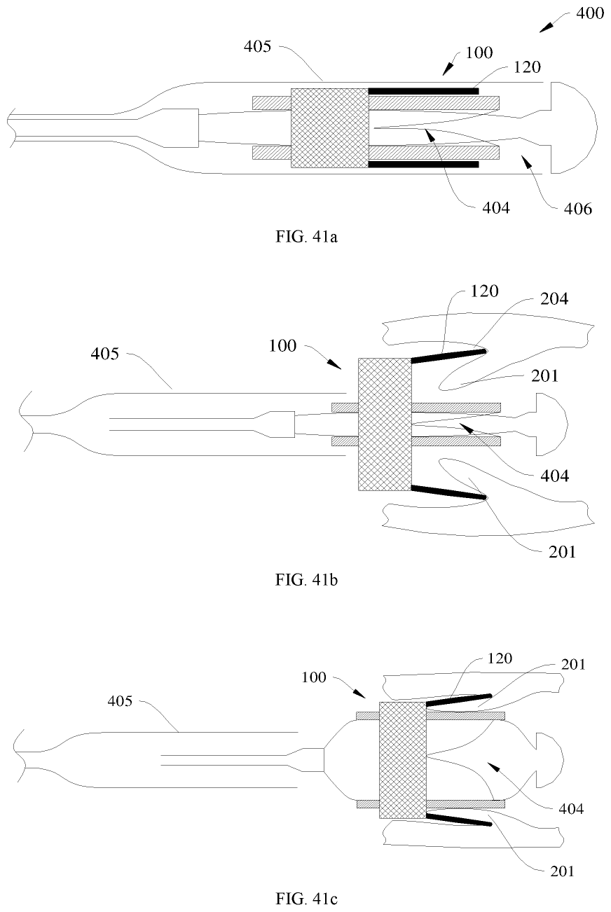

The specific operation of the prosthetic heart valve device 100 can be concluded referring to FIGS. 39a to 39b and the above description of the frame and would not be described here. The delivery process of the prosthetic heart valve device 100 will be described in detail below with respect to the delivery system.

The leaflets 200 and the covering film 220 can be any known repair material including processed animal tissue, such as pig tissue and bovine tissue, or synthetic material. The leaflets 200 and the covering film 220 can be attached to the frame 110 by conventional stitching.

Referring to FIGS. 41a to 41c, the present application further discloses a delivery system 400 for a prosthetic heart valve device 100, including:
a support device 404 that is switchable between the inflated and deflated configurations under fluid; and
an outer sheath 405 that is slidably engaged with the periphery of the support device 404, the radial gap between the outer sheath 405 and the support device 404 being a loading zone 406 for receiving the prosthetic heart valve device 100 in the compressed configuration.

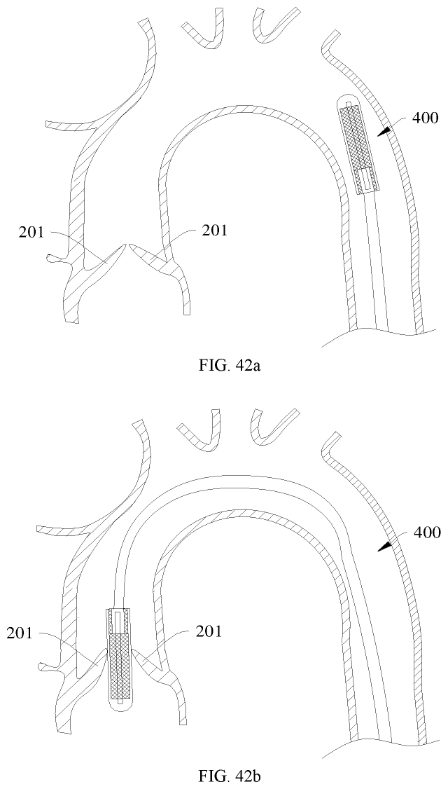

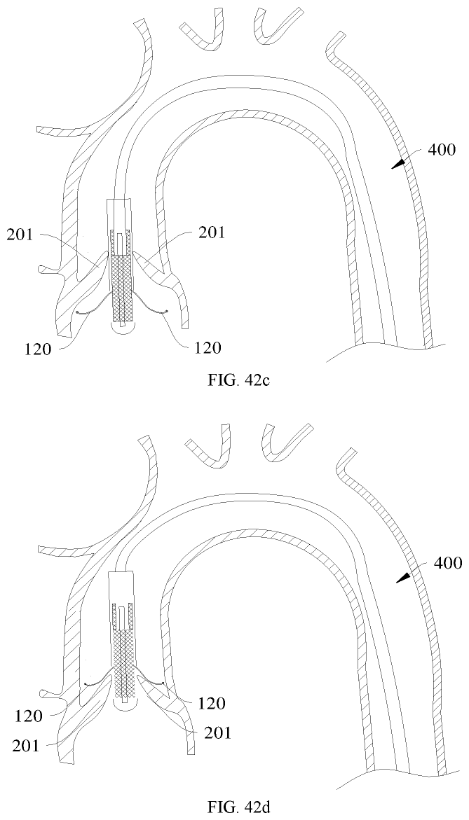

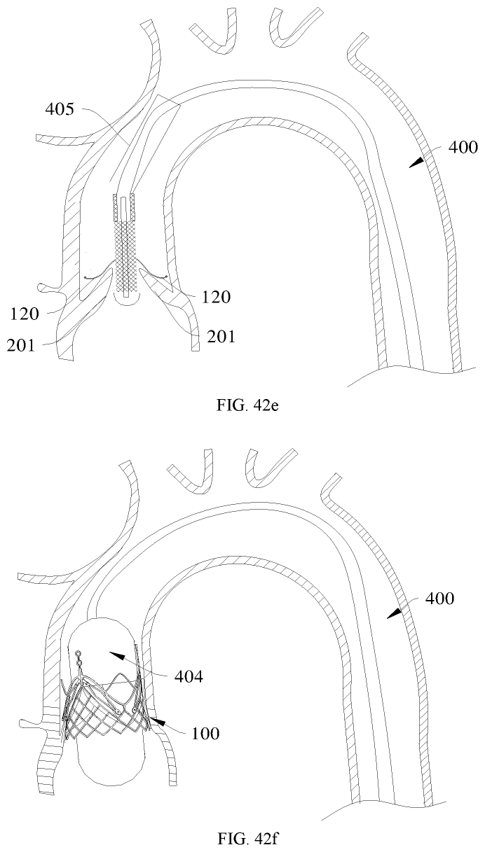

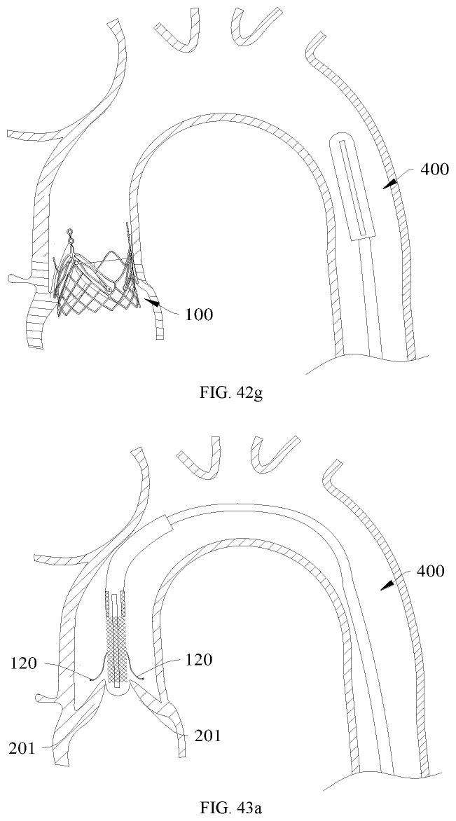

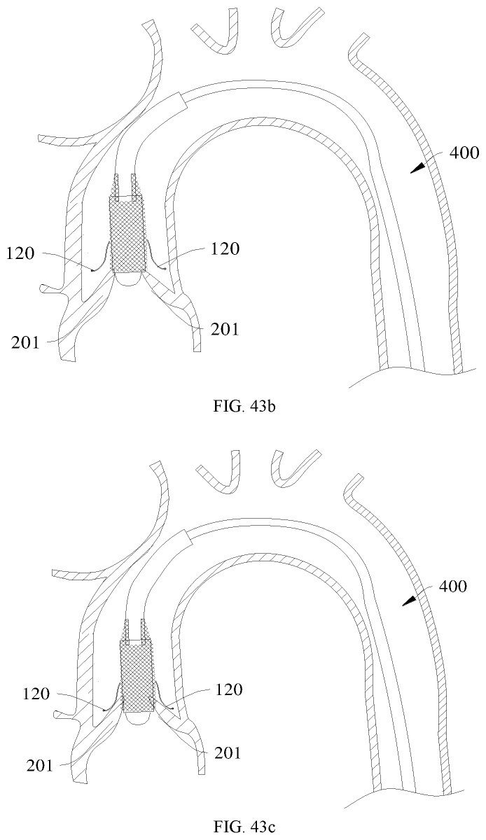

Referring to FIG. 42a to FIG. 43d, the present application further discloses a positioning method for the prosthetic heart valve device 100 for positioning any of the prosthetic heart valve devices 100 as described above, and the positioning method includes:
delivering the prosthetic heart valve device 100 to a predetermined site by a delivery system 400, in which the inner frame 103 is in a compressed configuration, the clipping arms 120 are in a loaded configuration, and the support device 404 is in a deflated configuration;
driving the outer sheath 405 to release the free ends 123 of the clipping arms 120, thereby expanding the free ends 123 of the clipping arms 120;
adjusting the position of the inner frame 103 such that the free end 123 of the at least one clipping arm 120 is located outside the native leaflet 201; and
driving the support device 404 to the inflated configuration and releasing the inner frame 103 and the fixed ends 121 of the clipping arms 120, so that the inner frame 103 transforms into the expanded configuration and the clipping arms 120 transform into the released configuration.

Optionally, before adjusting the position of the inner frame 103, the support device 404 is driven to a pre-inflated configuration, so that the inner frame 103 transforms into an intermediate configuration between the compressed configuration and the expanded configuration, and the clipping arms 120 transform into an intermediate configuration between the loaded configuration and the released configuration so as to achieve precise adjustment of the position of the inner frame 103.

The specific positioning method is explained in detail below with reference to the drawings.

Referring to FIGS. 42a and 42b, the delivery device delivers the inner frame 103 in a compressed configuration and the clipping arms 120 in a loaded configuration to a predetermined site. As shown in the figures, the delivery device passes through the native leaflets 201 after entering the target from the aortic arch. The specific puncture path can include the aortic or femoral artery or other feasible location.

Referring to FIGS. 42c-42d, the delivery device releases the clipping arms 120, causing the free ends 123 of the clipping arms 120 to expand. In this embodiment, the clipping arms 120 extend at the inflow end 101 of the native leaflets 201.

Referring to FIG. 42e, the position of the frame 110 is adjusted such that the free ends 123 of the clipping arms 120 are located just outside the native leaflets 201 while the inner frame 103 is located inside the native leaflet 201. In this embodiment, the position of the frame 110 is adjusted by withdrawing the delivery assembly, so as to improve the engagement of the clipping arms 120 and the native leaflets 201.

Referring to FIG. 42f, the support device 404 is driven to the inflated configuration, the inner frame 103 and the fixed ends 121 of the clipping arms 120 are released, so that the inner frame 103 transforms into the expanded configuration, and the clipping arms 120 transforms into the released configuration, wherein the inner frame 103 cooperates with at least one clipping arm 120 to hold the native leaflets 201.

Referring to FIG. 42g, the support device 404 is withdrawn and the delivery device is withdrawn. The transcatheter surgery is finished.

During the surgery, the expansion of the inner frame may affect the positioning of the clipping arms, which can be alleviated through specific operations.

Referring to FIGS. 43a to 43d, prior to adjusting the position of the inner frame 103, the support device 404 is driven to a pre-inflated configuration so that the inner frame 103 transforms into an intermediate configuration between the compressed configuration and the expanded configuration, and the clipping arms 120 transform into an intermediate configuration between the loaded configuration and the released configuration so as to achieve precise adjustment of the position of the inner frame 103. The inner frame 103 in the intermediate configuration allows to release the clipping arm 120 to a great extent, so that the intermediate configuration of the clipping arms 120 prior to adjusting the position of the inner frame 103 are closer to the completely released configuration thereof, thereby improving the positioning effect of the frame 110.

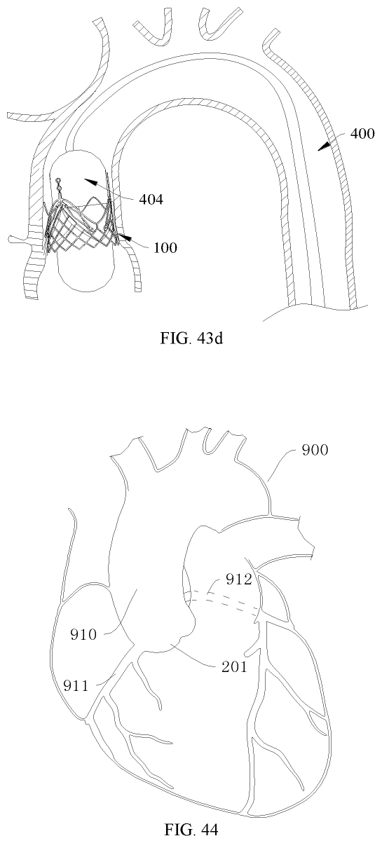

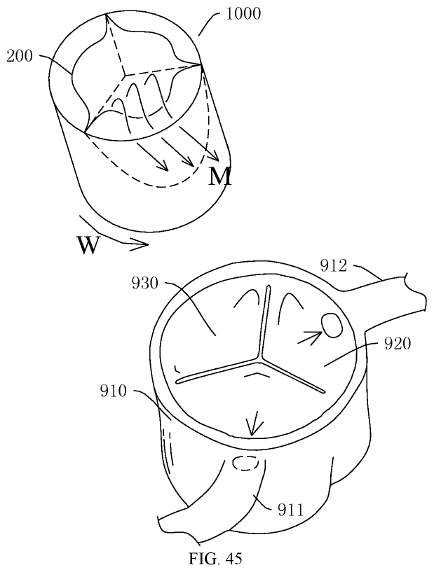

Referring to FIGS. 44-45, the aorta 910 of the human heart 900 is provided with three native leaflets 201, the valvular sinuses 204 are defined between the leaflets and the vessel wall, wherein two of the valvular sinuses respectively communicate with the right coronary artery trunk 911 and the left coronary artery trunk 912. The prosthetic aortic valve device 1000 should be positioned to ensure that blood flowing out through the opening among the leaflets 200 enters one of the main coronary arteries in the direction M. Therefore, the prosthetic aortic valve device 1000 should be displaced if the circumferential position thereof is offset. For example, in FIG. 45, the prosthetic aortic valve device 1000 can be rotated in direction W such that blood enters the left main coronary artery 912 in direction M.

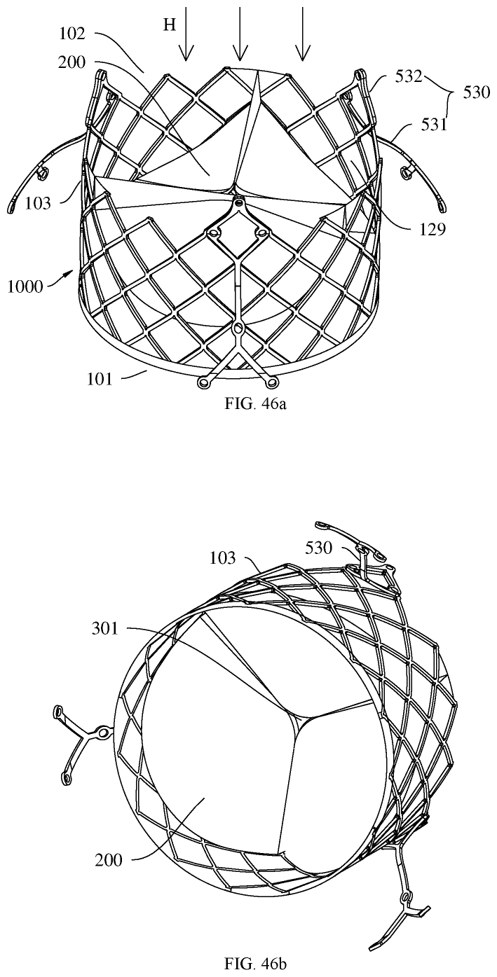

Referring to FIGS. 46a-52c, an embodiment of the present application provides a prosthetic aortic valve device 1000 having opposite inflow end 101 and outflow end 102, the prosthetic aortic valve device 1000 including:
an inner frame 103 having a meshed cylindrical structure, which is radially deformable and has relative compressed configuration and expanded configuration after being subjected to an external force, wherein the interior of the inner frame 103 is configured as an axially through blood flow channel 301, and the countercurrent blood flows in the direction H as shown in the figure;
leaflets 200 (prosthetic leaflets, in an opened state in FIG. 45) connected to the inner frame 103, wherein the leaflets 200 include three leaflets and cooperate to control the opening and closing of the blood flow channel 301; and
three guiding members 530 arranged in sequence in the circumferential direction of the inner frame 103 (the number of which corresponds to that of the aortic valvular sinuses), and the position thereof respectively aligned with the leaflets 200 in the circumferential direction, wherein each guiding member 530 includes a root 532 fixedly connected with the inner frame 103 and a wing 531 extending from the root 532 further toward the inflow end 101, the guiding member 530 is made of a memory material and is configured to be switchable between a loaded configuration, a transition configuration, and a released configuration.

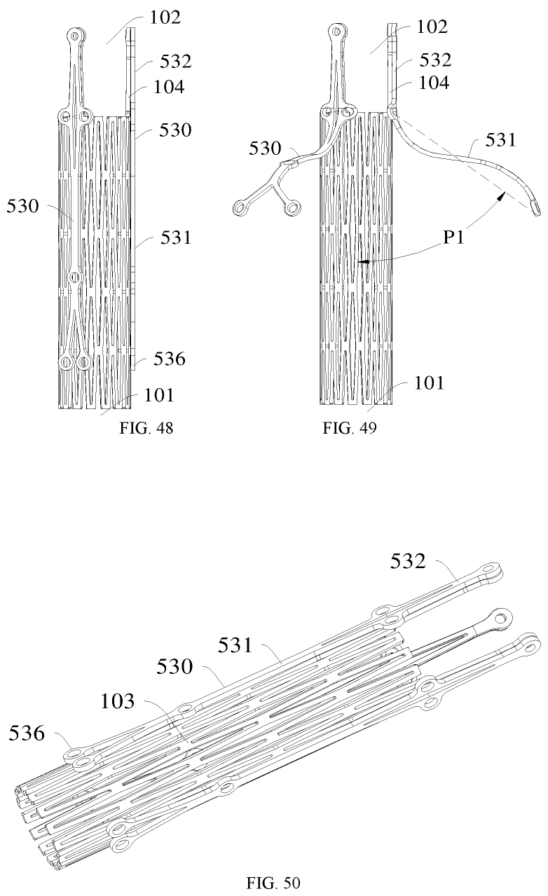

As shown in FIG. 48 and FIG. 50, in the loaded configuration, the inner frame 103 assumes the compressed configuration and the guiding members are radially pressed to contact the inner frame 103 in the compressed configuration, so that the inner frame 103 and the guiding members can be easily surrounded by the sheath and delivered in vivo.

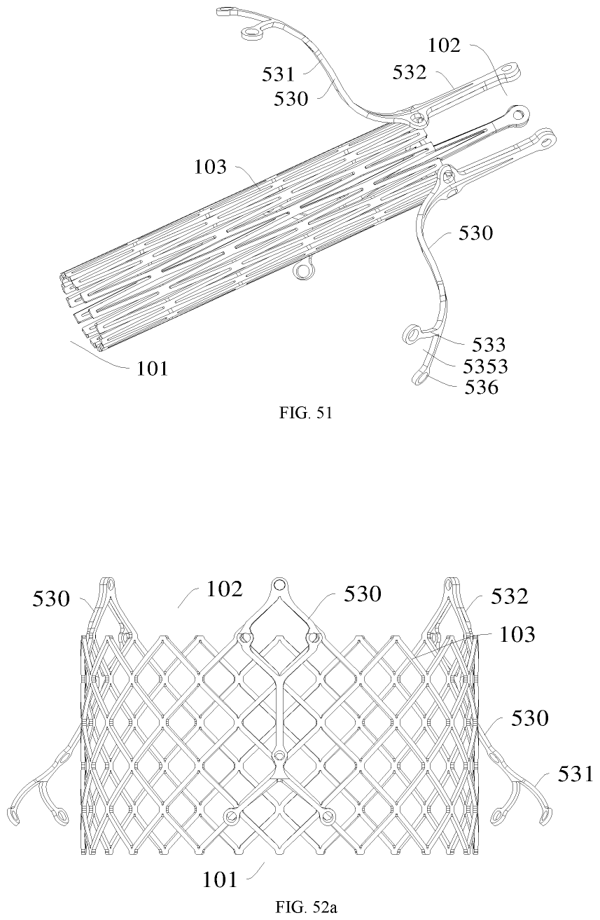

As shown in FIGS. 49 and 51, in the transition configuration, the inner frame 103 remains in the compressed configuration, and the roots 532 of the guiding members 530 remain gathered to adapt the compressed configuration of the inner frame 103. The wings 531 are self-deformed and thus extend outside of the inner frame 103, with an accommodation space formed between the outer wall of the inner frame 103 and the wings 531 for receiving the native leaflets 201. In order to circumferentially position the inner frame 103, the extended wings 531 can be adjusted in position to enter the corresponding valvular sinuses, with the inner frame 103 inside the native leaflets and the wings 531 outside the native leaflets.

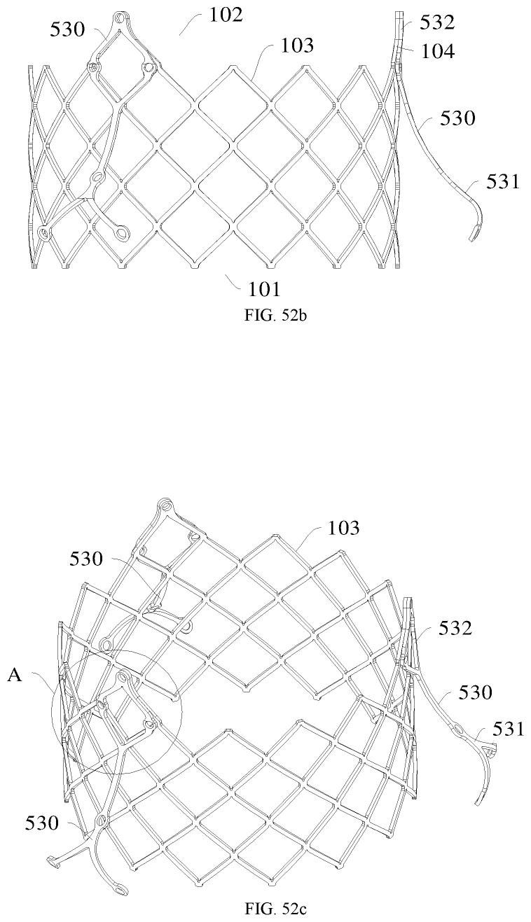

As shown in FIGS. 52a to 52c, in the released configuration, the inner frame 103 is already transformed into the expanded configuration after being subjected to an external force, and the roots 532 of the guiding members 530 move away from each other to adapt the expanded configuration of the inner frame 103.

In the present application, unless otherwise specified, the shape and position of the guiding member 530 are described referring to its released configuration, and the shape and position of the inner frame 103 are described referring to its expanded configuration.

The inner frame 103 has a meshed cylindrical structure, which can be radially deformed to facilitate the intervention after compression and the subsequent expansion and release. The axial length of the inner frame 103 may change when the inner frame 103 is radially deformed. The meshed cylindrical structure is configured to be expanded by external force, i.e., the meshed cylindrical structure is not made of self-expandable material. In general, the inner frame can be expanded by a balloon. However, the guiding member 530 is made of a memory material (e.g., pre-heat-set nickel-titanium alloy), the wing 531 of which can be released in the human body first, the root 532 of which can be considered as a portion where the guiding member 530 and the inner frame 103 are adjacent and connected to each other. The specific shape is not strictly limited. The root 532 and the wing 531 can be formed in one piece to facilitate processing. The wing 531 extends outward relative to the inner frame 103 after release, and by adjusting the posture of the inner frame 103, the wing 531 can enter into the valvular sinus 204 to pre-position the inner frame 103 in the circumferential direction, and then the inner frame 103 can be released and expanded by a balloon. Because the guiding members 530 are aligned with the valve leaflets 200, the junction of adjacent valve leaflets 200 avoids the coronary artery orifice and prevents the blood flow from being obstructed. In addition, the wings 531 abut against the bottoms of the valvular sinuses 204, which positions the inner frame 103 in the axial direction to avoid slipping to the left ventricle side under the action of the reverse flow of blood.

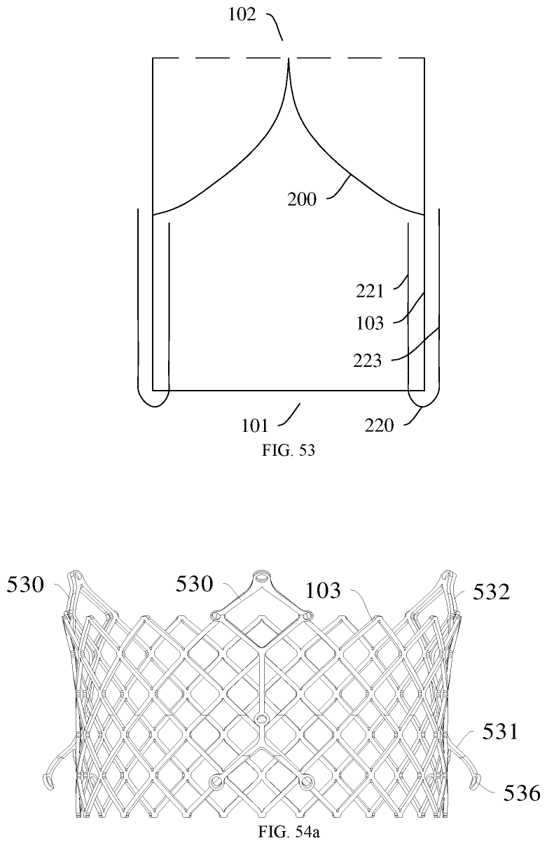

Referring to FIG. 53, in order to construct the blood flow channel and better fit with the surrounding tissue, the prosthetic aortic valve device 1000 further includes a covering film 220, which can include one or both of an inner covering film 221 and an outer covering film 223. The inner covering film 221 is fixed to the inner wall of the inner frame 103 and connected with the edge of the leaflets 200 at the inflow end 101, and the outer covering film 223 is fixed to the outer wall of the inner frame 103. Furthermore, the covering film 220 avoids the projection areas 129 of the leaflets 200 on the side wall of the inner frame.

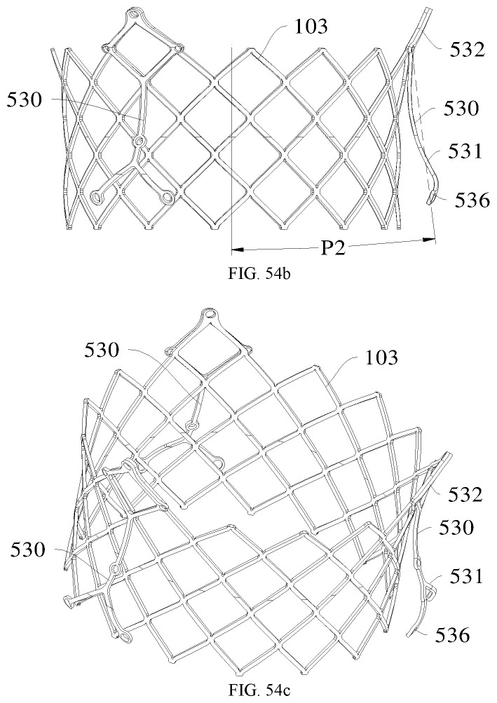

FIGS. 48 to 54c show the postures of the guiding members 530 in different configurations in the radial direction of the inner frame 103. In the loaded configuration, the guiding member 530 has the same diameter in the axial direction from the root 532 to the wing 531. In the transition configuration, the radial position of the root 532 of the guiding member 530 is unchanged, while the wing 531 is turned radially outward. In the released configuration, the guiding member 530 extends outwardly from the root 532 as the inner frame 103 expands, wherein the guiding member 530 extends radially outwardly and then is bent inwardly.

The guiding members 530 are made of a memory alloy, such as a pre-heat-set nickel-titanium alloy the shape of which corresponds to the released. The guiding member 530, at room or in-vivo temperature, has an internal stress in both the loaded configuration and the transition configuration relative to the released configuration. This internal stress urges the inner frame 103 and the guiding member 530 to switch to the final configuration in the body, and can be gradually eliminated as the inner frame 103 expands, so that the inner frame 103 and the guiding member 530 are better maintained in the final configuration. In the released configuration, the axial length of the guiding member 530 is 40% to 80%, for example, 50%, of the entire length of the inner frame 103.

The frame 110 generally includes the inner frame 103 and the guiding members 530. One end of the guiding member 530 away from the inner frame 103 is configured as the free end 536, and the root 532 can be regarded as a fixed end opposite to the free end 536.

In the loaded configuration, the wing 531 contacts the outer side of the inner frame 103. In the transition configuration, an angle P1 is defined between the wing 531 (referring to the line connecting the two ends of the wing) and the axis of the inner frame.

In the released configuration, the free end of the wing is closer to the out wall of the inner frame, with an angle P2 defined between the wing 531 and the axis of the inner frame, where P1 is great than p2. For example, P1 satisfies 30 to 60 degrees, and P2 satisfies 5 to 30 degrees. The free end of the wing closer to the outer wall of the inner frame can be caused by the outflow end of the inner frame turning outward, and by the shaping of the guiding member itself, separately or in combination.

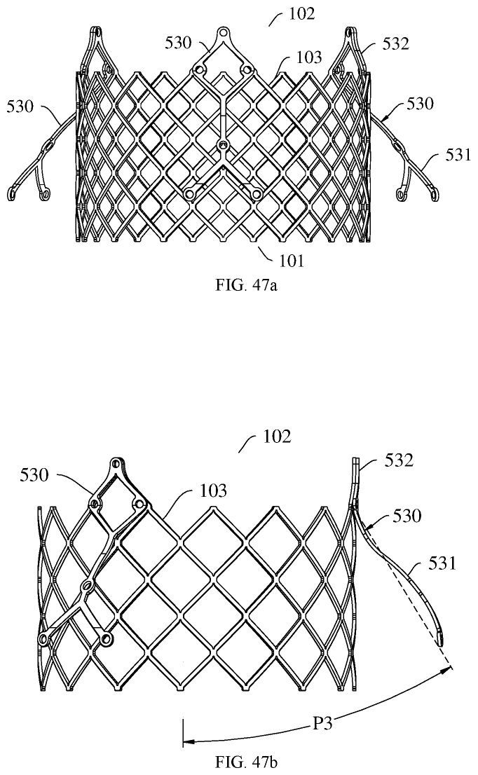

As shown in FIG. 47a and FIG. 47b, after the inner frame 103 is released, the inner frame 103 is still in a straight cylindrical shape, and an angle P3 is defined between the wing 531 and the axis of the inner frame, where P3<P1. The posture of the wing 531 shown in the figure is only for illustration, which does not strictly limit the angle.

As shown in FIGS. 55a to 55d, the inner frame 103 is formed by cutting a pipe material, and the material (for example, stainless steel) is suitable for balloon expansion release. The inner frame 103 has a straight cylindrical shape in the loaded configuration.

As shown in FIGS. 57a to 57d, in another embodiment, the outflow end 102 of the inner frame 103 is turned outward with respect to the axis, wherein the turning angle is P4 as shown in FIG. 57b, and P4 satisfies 0 degree<P4<45 degrees, such as 5 to 25 degrees.

The outflow end 102 slightly turning outward causes the free ends of the wings 531 to be closer to the inner frame 103 to clip the native leaflets and thus improve the positioning. The outflow end 102 can be turned by the balloon. For example, when the balloon extends beyond the outflow end of the inner frame 103, the balloon is released and thus tends to expand outward, thereby driving the outflow end 102 to turn outward. In the case where the axial length or the turning angle for the turning portion is further increased, the frame 110 can be shaped to turn outward directly using the expanded end portion of the balloon.

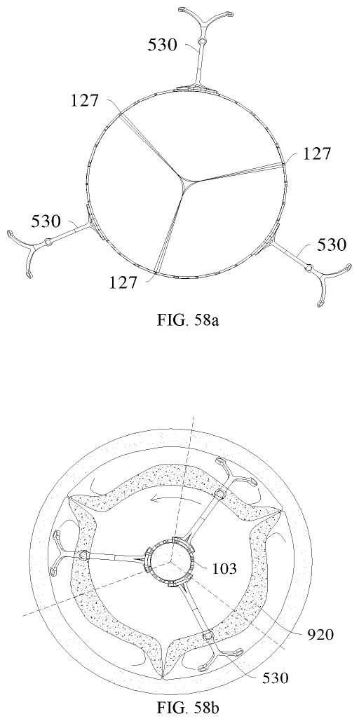

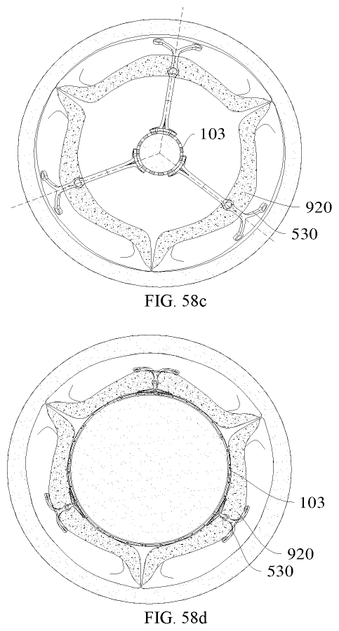

In order to fix the guiding member 530, as shown in FIG. 58a, two adjacent leaflets 200 are connected on the inner frame 103 at the connecting portion 127 of the inner frame 103, and the root 532 of the guiding member 530 is located between two adjacent connecting portions 127, but is not limited to being strictly centered therebetween.

The guiding member 530 is generally configured as a bar. Each guiding member 530 is formed in one piece and switches the configurations thereof based on its own elastic deformation. Compared with a hinge structure, the internal stress of the guiding member of the present application can be used as the driving force for deformation. Referring to FIGS. 58b and 58c, after release of the guiding members 530, there may be deviations in the circumferential positions of the guiding members 530 from the positions of the valvular sinuses 204. For example, the areas represented by the three radially extending solid lines can be regarded as the approximate distribution areas of the three guiding members, while the areas represented by the three radially extending dotted lines can be regarded as the approximate distribution areas of the three valvular sinuses, which are not aligned with each other as shown in FIG. 58b, in which case, the inner frame 103 can be rotated in the direction of the solid arrow shown in the figure to drive the guiding members 530 until the three radially extending solid lines coincide with the dashed lines, so as to achieve circumferential alignment as shown in FIG. 58c.

After circumferential alignment, the inner frame 103 is moved toward the inflow end until the guiding members 530 abut against the bottom of the valvular sinuses 204 or the native leaflets have filled the accommodation space between the inner frame 103 and the guiding members 530 to achieve positioning. FIG. 58d show the axial position, and FIGS. 59a to 59c show the radial position, wherein the native leaflet 201 is located between the guiding member 530 and the inner frame 103. The inner frame 103 can be then released and expanded by a balloon, thereby avoiding the coronary artery.

Referring to FIG. 59b, when the balloon 630 is expanded, the ends of the inner frame 103 first tend to turn over, during which process, the free ends of the guiding members 530 will tend to move inward and begin to clip the native leaflets 201. The inner frame 103 is completely released radially at the later stage of the balloon expansion. As shown in FIG. 59c, since the root of the guiding member 530 deforms circumferentially with the deformation of the inner frame, the free end of the guiding member 530 is further moved toward the inner frame 103 to clip the native leaflet. The mechanism of deformation of the guiding member 530 is further described below.

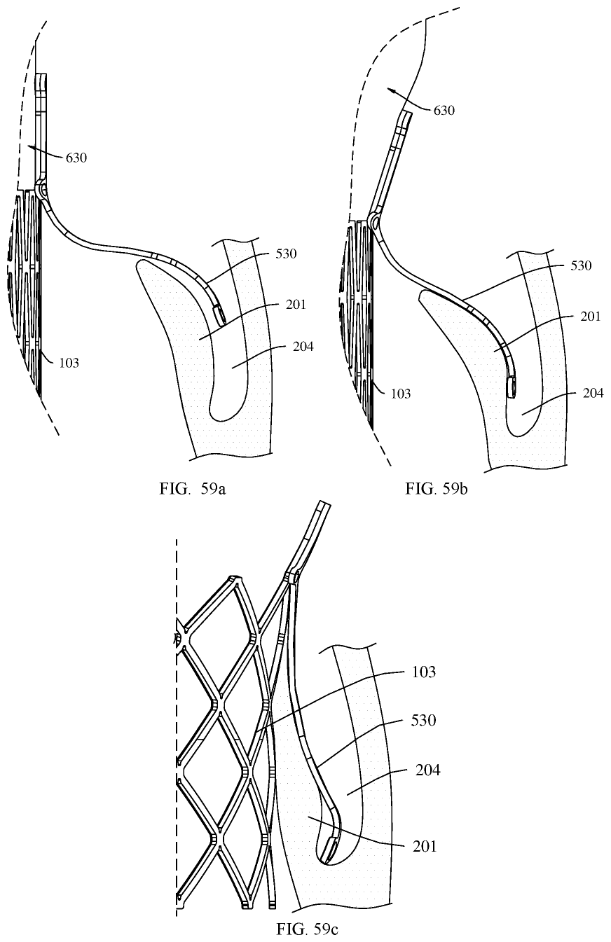

Referring to FIGS. 60a-62c, the wing 531 is a branched structure 535 adjacent to the root 532, the end of the wing 531 away from the root 532 is configured as a free end 536, and the slot 5353 of the branched structure 535 is toward the outflow end 102. The branched structure 535 converges and extends toward the free end 536.

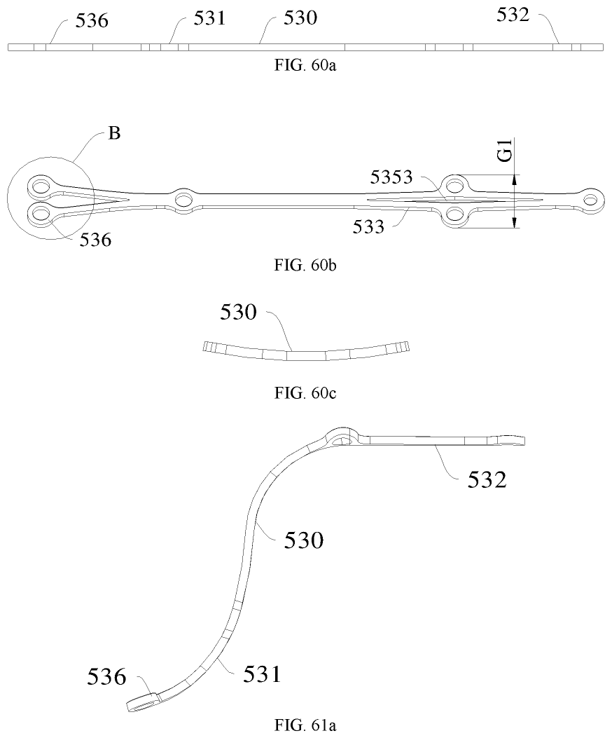

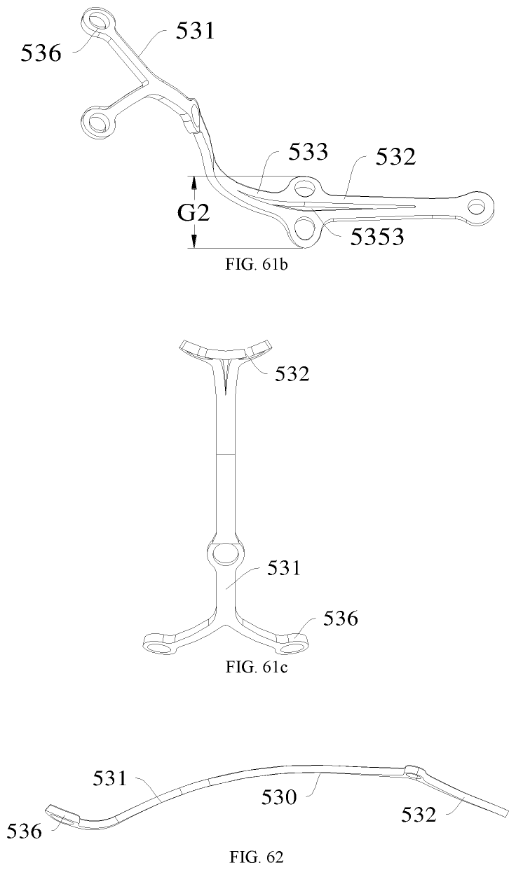

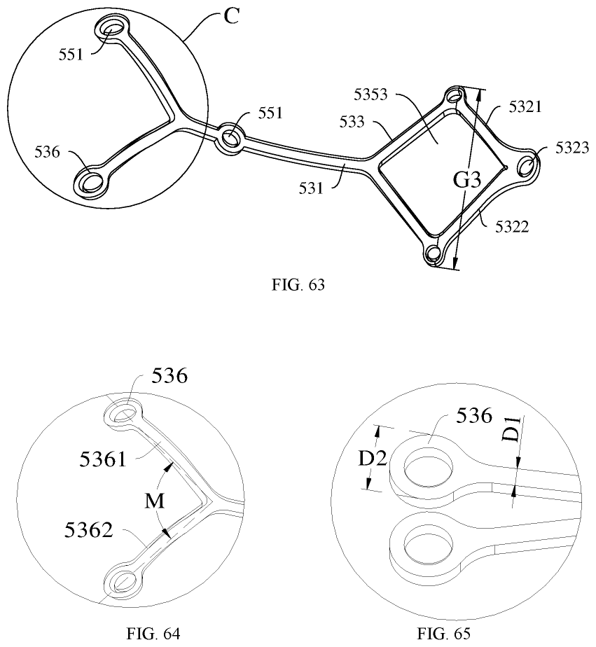

As shown in FIGS. 60a-61c, the two opposite parts of the branched structure 535 are constrained by the root 532 to move toward each other in the loaded configuration and in the transition configuration. As shown in FIGS. 62a-62c, the two opposite parts of the branched structure 535 move away from each other with the deformation of the root 532 and the inner frame 103 in the released configuration. For example, in the loaded, transition, and released configurations, the circumferential spans of the two opposite parts of the branched structure 535 are G1, G2, and G3, respectively, satisfying G1=G2<G3.

The root 532 of the branched structure 535 forms as a triangle, a trapezoid, or a rectangle, or the like. The two opposite parts of the branched structure 535 converge and extend toward the free end 536, and then split circumferentially adjacent the free end 536, where the branched structure 535 is divided into at least two parts, i.e., the seventh bar 5361 and the eighth bar 5362, respectively, and the angle M between the connecting lines of the respective ends of the two bars is about 45 degrees or more, for example, 45 to 120 degrees as shown in FIG. 64. In the axial direction of the inner frame 103, the free end 536 is located adjacent to the inflow end 101 of the inner frame 103, and the root 532 is located adjacent to the outflow end 102 of the inner frame 103, so that the wing 531 has a sufficient extension to ensure positioning. In order to improve safety, the free end 536 has a rounded structure, and can be further surrounded with a protective layer. Alternatively, the free end 536 can be ring-shaped, and can be further covered with protective layer or can be suffered from a surface smoothness treatment.

As shown in FIG. 65, the wing 531 has opposite length and width directions, and the width of the ring-shaped free end is larger than the width of the wing bar. The width D2 of the ring-shaped free end is 2 to 6 times the width DI of the wing bar.

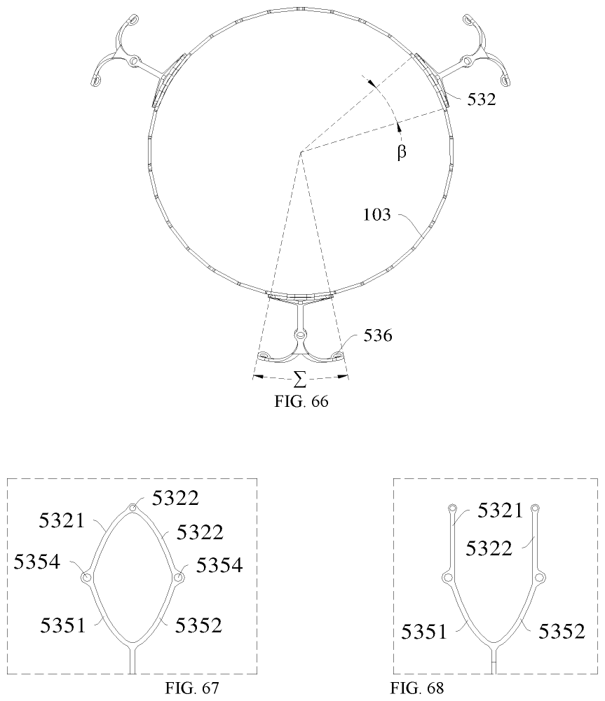

As shown in FIG. 66, in order to better position the inner frame 103 and reduce the offset after positioning, the guiding members 530 need to have a sufficient circumferential span. The circumferential span of the single guiding member 530 has a center angle X of 30 to 60 degrees, and the circumferential span P of the root 532 of the single guiding member with respect to the inner frame 103 is 15 to 45 degrees.

The wing 531 expands radially outward and then bends inward during the extension to the inflow end, providing greater clipping force and allowing greater radial deformation.

The root 532 is fixed to the radially inner, or outer side of the inner frame 103 or radially aligned with the inner frame 103 by means of welding, riveting or binding, so that the root 532 is always attached to the inner frame 103 in any configuration, and deforms in the circumferential direction as the inner frame 103 deforms in the circumferential direction in the transition configuration and the released configuration, wherein the deformation amount of the root 532 is the same as the corresponding portion of the inner frame.

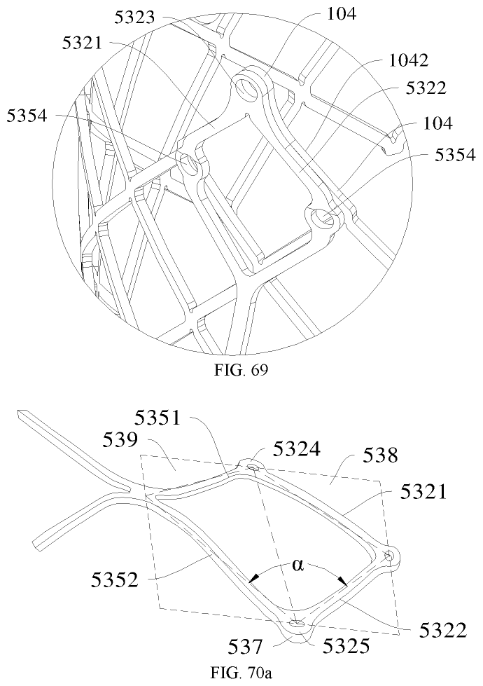

The root 532 can be secured to the outside of the inner frame 103 by binding to facilitate assembly and allow the bars of the root 532 to twist about its own longitudinal axis. The root 532 includes a first bar 5321 and a second bar 5322 connected to the wing 531 (i.e., a branched structure). The root 532 further extends toward the outflow end 102 relative to inner frame 103 to provide sufficient space to allow the inner frame 103 to be lowered further in position, ensuring that the free end of the wing extends into the bottom of the valvular sinus. As shown in FIGS. 67 to 69, the ends of the first bar 5321 and the second bar 5322 away from the wing 531 are connected with, parallel to or away from each other.

For example, the ends of the first bar 5321 and the second bar 5322 away from the wing 531 are connected with each other, and are fixed to the inner frame 103 through a binding line (not shown) passing through the first binding eyelet 5323. Similarly, the corresponding portion of the inner frame can also be provided with a similar eyelet as required.

The other ends of the first bar 5321 and the second bar 5322 are spaced apart from each other and are connected to the wing 531 (i.e., the branched structure) to form a closed quadrangle. In order to facilitate the positioning and threading, one end of the first and second bars connecting with the wing 531 is respectively provided with a second binding eyelet 5354. Similarly, the corresponding portion of the inner frame can also be provided with a similar eyelet as required.

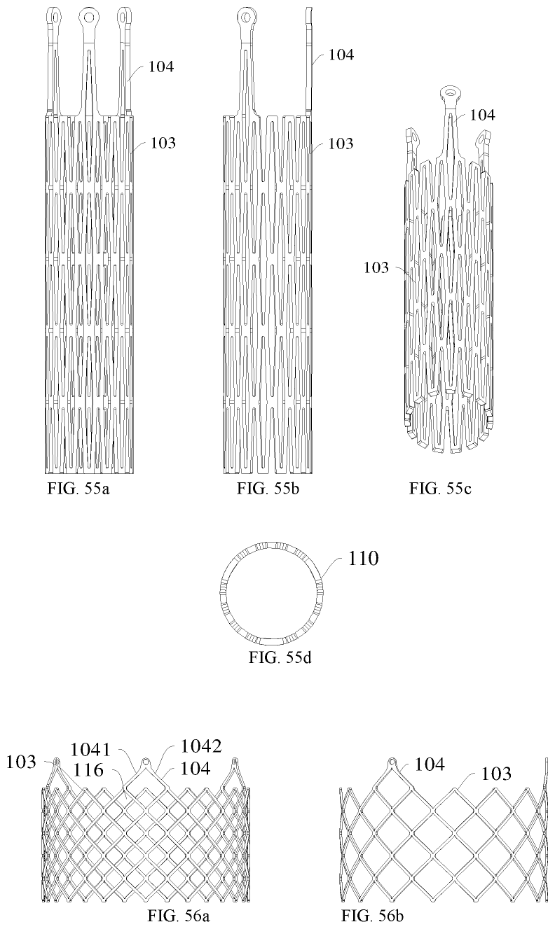

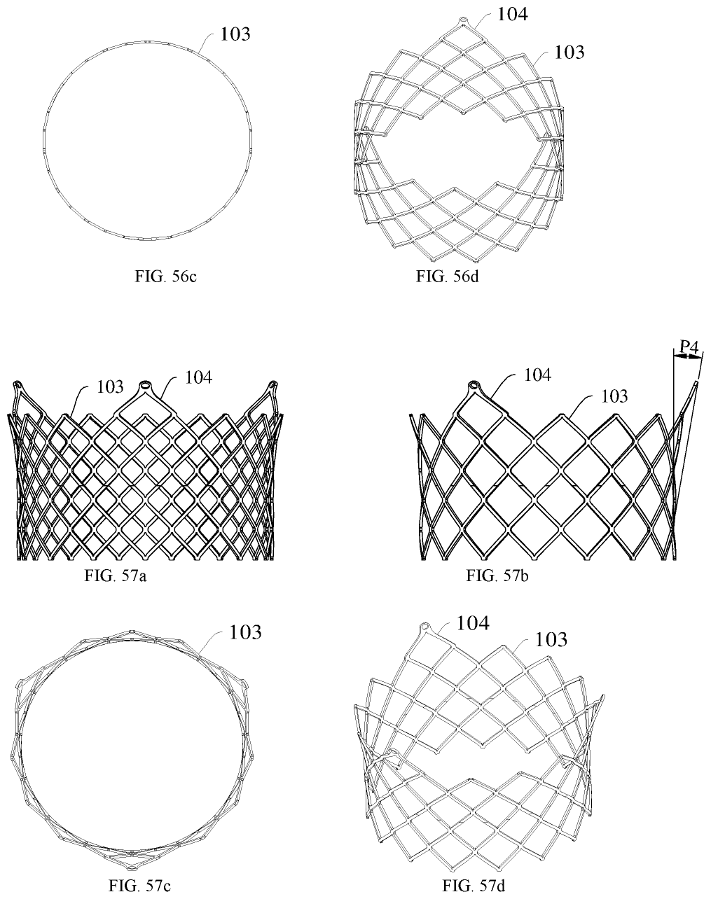

In some embodiment, the inner frame 103 has a connecting post 104 extending axially and outwardly toward the outflow end 102. The connecting post 104 can use the same shape as the root 532 and radially overlap on the inner frame. The same shape means that the connecting post 104 also includes fifth bar 1041 and sixth bar 1042 similar to the first bar 5321 and the second bar 5322 (in combination with FIG. 56a). The fifth and sixth bars 1041, 1042 conform to the shape of the root 532, for example, the ends thereof adjacent the outflow end 102 meet each other such that the tip of the connecting post 104 is V-shaped toward the outflow end 102, or parallel, or are parallel to or away from each other. The first bar 5321 and the fifth bar 1041 can be overlap with each other, and the second bar 5322 and the sixth bar 1042 can be overlap with each other.

The inner frame 11 has a plurality of diamond shaped cells 116 distributed in the axial direction, the root 532 of the single guiding member 530 corresponds to one or two cells with respect to the circumferential span of the inner frame 103. As shown in FIG. 56a, the fifth bar 1041 and the sixth bar 1042 extend from the end nodes of the inner frame 103. The cells of the inner frame at the outflow end 102 are cut in half and thus opened, and the ends of the fifth bar 1041 and the sixth bar 1042 are connected with two adjacent cells 116.

The wing 531 includes a third bar 5351 and a fourth bar 5352 adjacent the root and thus forms a branched structure, wherein one end of the third bar 5351 is connected with the first bar 5321, one end of the fourth bar 5352 is connected with the second bar 5322, and the other ends of the third bar 5351 and the fourth bar 5352 extend toward the inflow end 101 and intersects with the third bar 5351.

The first bar 5321, the second bar 5322, the third bar 5351, and the fourth bar 5352 form a closed region, and the radially projected shape of which is quadrangular. For example, the four bars form a parallelogram.

In the case where the ends of the first bar 5321 and the second bar 5322 away from the wing 531 are parallel to or away from each other, the first bar 5321 and the second bar 5322 as well as the wing form a semi-closed area opened toward the outflow end 102.

The above bars are not strictly limited to be straight bars, but can be slightly curved or bent. The fourth bar 5352 and the third bar 5351 can be directly connected with each other or indirectly connected by other bar(s). As shown in the figure, the fourth bar 5352 and the third bar 5351 are directly connected with each other. After the bars are connected with each other, the bar can extend a certain distance and then be branched to the free ends, or be directly branched to the free ends, or can be branched and then meet again to form a ring structure, which can reduce the interference on the coronary orifice and the risk of puncturing the tissue.

The junction between two adjacent bars, for example, between the fourth bar 5352 and the second bar 5322, does not need a sharp turning, but can be shaped smoothly. For example, the third bar 5351 and the fourth bar 5352 can be formed in one piece having an arc structure, wherein the third bar 5351 and the fourth bar 5352 represent different portions of the arc structure. Therefore, it can be conceived that the first to fourth bars above can not only form as a parallelogram, but also can be an enclosed circle, ellipse, or even hexagon or the like.

At least the third bar 5351 is not collinear with the first bar 5321, and the fourth bar 5352 is not collinear with the second bar 5322, otherwise, the expected deformation of the guiding member would be affected or weakened.

In the following, the deformation of the guiding member when it is switched between the transition configuration and the released configuration will be explained, wherein the first bar 5321 and the second bar 5322 define a first portion 538, and the third bar 5351 and the fourth bar 5352 define a second portion 539.

The third bar 5351 and the first bar 5321 meet at a first connection point 5324; the fourth bar 5352 and the second bar 5322 meet at a second connection point 5325; the first bar 5321 and the second bar 5322 meet at a third connection point 5326; and the third bar 5351 and the fourth bar 5352 meet at a fourth connection point 5355, wherein the first connection point 5324, the second connection point 5325, and the third connection point 5326 form a first plane 5327 in which the first portion is located, and the first connection point 5324, the second connection point 5325 and the fourth connection point 5355 form a second plane 5356 in which the second portion is located. It should be noted that the first plan and the second plane are for illustration, and they may be slighted curved or approximate planes.

The structure enclosed by the first to fourth bars above does not need to correspond to the cell of the inner frame. For example, the first connection point 5324 and the second connection point 5325 can be respectively aligned with the nodes of the inner frame, or can be offset from the nodes of the inner frame to reduce the interference with the inner frame during deformation.

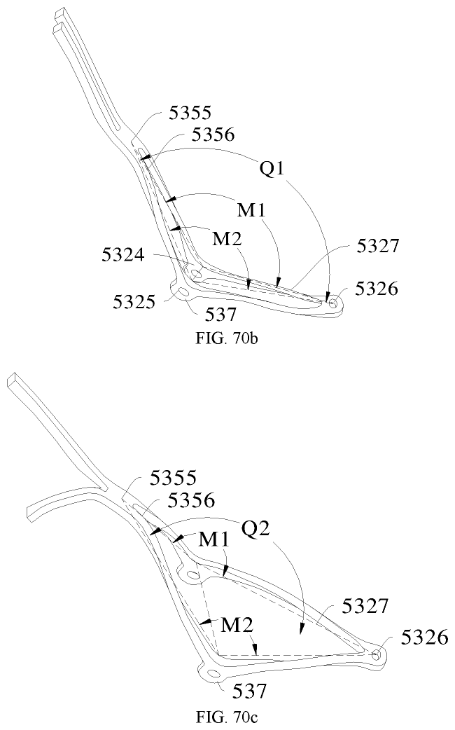

In FIG. 70a, when the bars are fully extended, they lie in the same plane (Q=180 degrees), and the distance between the first connection point 5324 and the second connection point 5325 is at the largest.

In FIG. 70b, in the transition configuration, the inner frame 103 is in the compressed configuration, so that the first connection point 5324 and the second connection point 5325 are close to each other, the wing 531 is warped with respect to the root 532, and the first plane 5327 and the second plane 5356 form an angle QI therebetween. It should be noted that, when the first connection point 5324 and the second connection point 5325 moves toward each other, each bar may twist about its own axis in order to adapt the warpage of the wing 531, otherwise, the deformation only occurs in a plane, i.e., only the length of the guiding member is stretched, which is in cooperation with the binding of the root to the inner frame 103. It can be seen from the figures that, in different configurations, the first to fourth bars have already twisted, and the first connection point 5324 and the second connection point 5325 are no longer coplanar with the third connection point 5326 and the fourth connection point 5355.

In FIG. 70c, when transforming from the transition configuration to the released configuration, the first connection point 5324 and the second connection point 5325 move away from each other, and the warpage degree of the wing 531 is reduced, in which case, the angle between the first portion 538 and the second portion 539 is Q2, and QI is less than Q2, which means that the free end of the wing is closer to the inner frame, facilitating clipping the native leaflets.

It can be seen from the figures that the angle M1 between the axis of the third bar 5351 and the axis of the first bar 5321 and the angle M2 between the second bar 5322 and the fourth bar 5352 are substantially unchanged when switching between the transition configuration and the released configuration. For example, M1=M2=120 degrees. In other words, the guiding member is not deformed in a plane, but in three dimensions.

As described above, it can be seen that the root 532 and the portion of the wing 531 connected with the root 532 constitute a frame structure, for example, including the first to fourth bars. Two ends of the frame structure in the circumferential direction, for example, the first connection point 5324 and the second connection point 5325, are relatively turned over as the inner frame is compressed and expanded, thereby driving the two ends of the frame structure in the axial direction of the inner frame, such as the third connection point 5326 and the fourth connection point 5355, to be relatively turned over.

In the frame structure, when the two ends in the axial direction of the inner frame are turned over relative to each other, one end such as the third connection point 5326 is fixed relative to the inner frame, and the other end such as the fourth connection point 5355 is turned over relative to the outer wall of the inner frame.

In the frame structure, when the two ends in the circumferential direction of the inner frame are turned over relative to each other, both ends such as the first connection point 5324 and the second connection point 5325 are turned over relative to the outer wall of the inner frame.

In order to facilitate the deformation, during processing the guiding member, the wing 531 can be slightly warped with respect to the root 532 in the shaping configuration after the heat treatment.

The guiding member 530 has restricting structures 537 opened at the first connection point 5324 and the second connection point 5325, and the first connection point 5324 and the second connection point 5325 are bound to the inner frame 103 through the restricting structures 537. The restricting structure 537 can be configured as an eyelet (i.e., the second binding eyelet 5354) or other protrusions extending circumferentially outwardly with an eyelet. The restricting structure 537, as a force point, rotates relative to the axis of the bar, thereby driving the portions of the bars adjacent to the restricting structure to twist.

In order to reduce the restraint on the twist of the bars and obtain a larger turning angle of the wing, when binding, only one side with the eyelet in the axial direction of the inner frame is bound, as the bars may be restrained to twist if two sides in the axial direction of the inner frame are bound.

In cooperation with an imaging equipment, the prosthetic aortic valve device 1000 can be provide with a developing marker 550, which can be embedded or include a precious metal that can be displayed differentiating from other portions under X-ray or other means of detection.

The developing marker 550 can be in the form of a dot or a strip or a ring (closed or non-closed, but at least in half ring), and the developing marker 550 can be disposed on at least one of the inner frame 103 and the guiding members 530. Accordingly, the inner frame 103 or the guiding members 530 are provided with eyelets for receiving the developing marker 550.

Optionally, each of the above binding eyelets can be provided with a developing marker, or the developing marker can be provided at the middle portion or the free end of the wing.

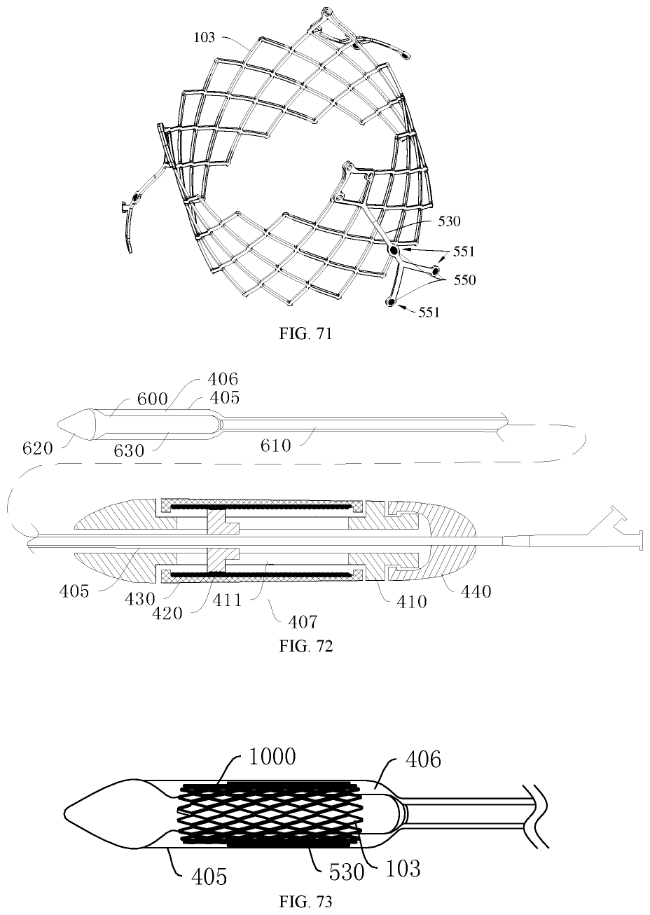

For example, as shown in FIG. 71, the free end 536 carries developing markers 550. The free end 536 has eyelets 551 at which the developing markers are located. As another example, the wing 531 is provided with an eyelet 551 at a position before being branched and can be provided with a developing marker at the eyelet 551.

Referring to FIGS. 72 and 73, in one embodiment, a delivery system for a prosthetic aortic valve device 1000 is provided that can be used to load and deliver the prosthetic aortic valve devices 1000 of the above embodiments. The delivery system has opposite distal and proximal ends, the delivery system including:
a balloon device 600 switchable between an inflated configuration and a deflated state under the action of a fluid;
an outer sheath 405 which is slidably fitted on the outer periphery of the balloon device 600, and a radial gap between the outer sheath 405 and the balloon device 600 is a loading zone 406 for placing the prosthetic aortic valve device 1000; and
a control handle 407, wherein both the proximal ends of the balloon device 600 and the outer sheath 405 extend to the control handle 407 with the outer sheath 405 slidably fit with the control handle 407.

The outer sheath 405 can be moved to cover or expose the prosthetic aortic valve device 1000 to effect switching between the loading and delivery configuration and release configuration. In the delivery system, the outer sheath 405 and the balloon device 600 are rotatably fitted with each, that is, the circumferential position of the prosthetic aortic valve device 1000 can be adjusted by rotating the balloon device 600 so that the valve leaflets 200 can be aligned with the valvular sinuses. In addition, the prosthetic aortic valve device 1000 of the present embodiment is provide with guiding members 530, a developing marker(s) 550 is provided on one of the inner frame 103 and the guiding members 530, so that the prosthetic aortic valve device 1000 can be monitored in real time by mean of an imaging equipment when the position thereof is adjusted, so as to guide the surgery. In this embodiment, the arrangement of the guiding member 530 and the developing marker 550 in cooperation with the rotation of the outer sheath 405 and the balloon device 600 ensures accurate positioning of the prosthetic aortic valve device 1000.

In some case, for example, where the balloon device 600 cannot be rotated relative to the outer sheath 405, although the balloon device 600 and the outer sheath 405 can be rotated together for circumferential alignment, the outer sheath 405 will twist itself due to a relatively long intervention length, and it is difficult for the outer sheath 405 to recover to its untwist configuration around its own axis, so that a large force would inevitably occur between the outer sheath 405 and the surrounding tissues. However, in this embodiment, the outer sheath 405 is used for provide a stable passage, the rotatable balloon device 600 (the tube inside the outer sheath 405) can be twisted around its own axis in the passage so as to reduce the risk to the maximum extent.

When the balloon device 600 is rotated, the outer sheath 405 can be kept at least from being excessively twisted in the circumferential direction, and can be reinforced as needed, for example, by means of an inner rib, a reinforcing mesh, a hypotube, or the like.

The aortic prosthetic valve device 1000 as a whole is radially compressed and placed in the loading zone 406 and surrounded within the distal section of the outer sheath 405. In the release process, the guiding members 530 are progressively exposed by sliding the outer sheath 405 proximally. At this time, although the inner frame 103 is exposed, the inner frame 103 cannot automatically transform into the expanded configuration due to its material thereof, and the circumferential position of the inner frame 103 can be aligned by rotating the balloon device 600. After alignment, the inner frame 103 is driven to expand using the balloon device 600. During the alignment, the outer sheath 405 can be kept relatively stationary, reducing safety hazards and improving the alignment.

Referring to FIG. 72 and FIGS. 73 to 76, the balloon device 600 includes:
a tube 610 having at least a guide wire passage and an injection channel provided therein, the proximal end of the tube 610 being rotatably mounted to the control handle 407;
a guide head 620 which is fixed to the distal end of the tube 610, the distal end of the guide wire passage is opened into the guide head 620, and wherein during the delivery of the delivery system in vivo, the guide wire can be first intervened into the human body, and then the entire delivery system can be surrounded around the guide wire through the guide wire passage and is advanced along the guide wire; and
a balloon 630 fixed to the tube 610 at the proximal side of the guide head 620, the interior of the balloon 630 communicating with the injection channel.

The guide wire passage and the injection channel can be provided with additional tubes, or by a multi-lumen tube, and the guide wire passage and the injection channel can be respectively provided with a tube connector (for example, a three-way structure on the right side shown in FIG. 72, such as a luer connector or the like) at the proximal ends thereof. In practice, the injection channel can be used to deliver fluid to inflate the balloon 630.

In order to allow rotation of that balloon device 600, the tube 610 should be capable of ensuring circumferential torque transmission and minimizing angular deviation between the distal and proximal end. For example, the tube 610 can include a multi-layer structure from the inside to the outside, and at least one layer in the middle is provided with embedded ribs, reinforcing mesh, hypotubes, steel cables and the like to ensure the synchronization of the proximal and distal ends. Of course, when there is a deviation, correction and real-time adjustment can be further carried out by means of the developing marker.

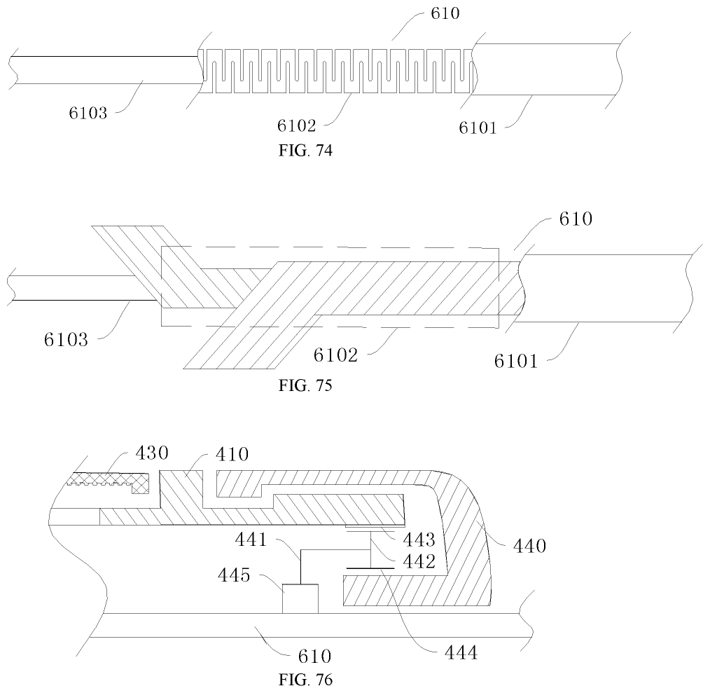

For example, as shown in FIG. 74, in one embodiment, the tube 610 has a three-layer structure, the middle layer 6102 is a hypotube and is between the outermost layer 6101 and the innermost layer 6103, and the outermost layer 6101 and the innermost layer 6103 can be made of conventional materials such as Pebax and TUP, which are respectively fixed to the hypotube by means of thermal fusion or the like. The cutting method of the hypotube is not strictly limited, for example, alternate slits at different circumferential positions can be provided to provide the compliance for passing through a curved intervention path.

For example, as shown in FIG. 75, in another embodiment, the middle layer 6102 is made of two layers of steel cable tubes wound in opposite directions, which have compliance and can ensure the transmission of torque in the circumferential direction.

The control handle 407 includes:
a support 410;
a movable base 420 movably mounted on the support 410, to which the proximal end of the outer sheath 405 is fixed;
a driving sleeve 430 rotatably mounted on the outer periphery of the support 410 and engaged with the movable base 420 to drive the outer sheath 405 to slide relative to the balloon device 600; and
a rotatable seat 440 rotatably mounted on the outer periphery of the support 410 and engaged with the tube 610 of the balloon device 600 to drive the balloon device 600 to rotate relative to the outer sheath 405.

The driving sleeve 430 and the movable base 420 are threadably engaged with each other. The rotation of the driving sleeve 430 can drive the movable base 420 to slide. In order to prevent free rotation of the movable base 420, the support 410 is provided with a guiding structure, such as a sliding groove 411 or a guiding rod, for restricting the movement of the movable 420. The outer periphery of the support 410 can be fixedly covered with a shell, so as to play a protective and aesthetic role.

The rotatable seat 440 can be directly fixed to the tube 610 of the balloon device 600 as shown in FIG. 72. In operation, the rotatable seat 440 is directly operated, and a marker can be arranged on the rotatable seat 440 and the support 410 to show the direction and magnitude of rotation of the rotatable seat 440.

The rotatable seat 440 and the balloon device 600 can be indirectly connected by a transmission mechanism. A speed reduction mechanism can be used to improve the accuracy of adjustment and improve the feel.

Referring to FIG. 76, this embodiment employs a planetary reduction mechanism, specifically including a planetary carrier 441, planetary gears 442, a ring gear 443, a planetary input shaft 444, and a planetary output shaft 445. The planetary input shaft 444 has external teeth and is fixed to the rotatable seat 440 and configured to be driven by the rotatable seat 440. The planetary input shaft 444 and the rotatable seat 440 can be formed in one or separate pieces.

The ring gear 443 has internal teeth and is fixed to the support 410. The ring gear 443 and the support 410 can be formed in one or separate pieces. The planetary gears 442 generally include three planetary gears 442, meshing between the planetary input shaft 444 and the ring gear 443 and configured for driving the planetary carrier 441. The planetary carrier 441 is fixed to the planetary output shaft 445. When the planetary gears 442 revolve, the planetary carrier 441 rotates, and then the planetary output shaft 445 drives the fixed tube 610 to rotate, thereby driving the balloon device 600 to rotate the inner frame.

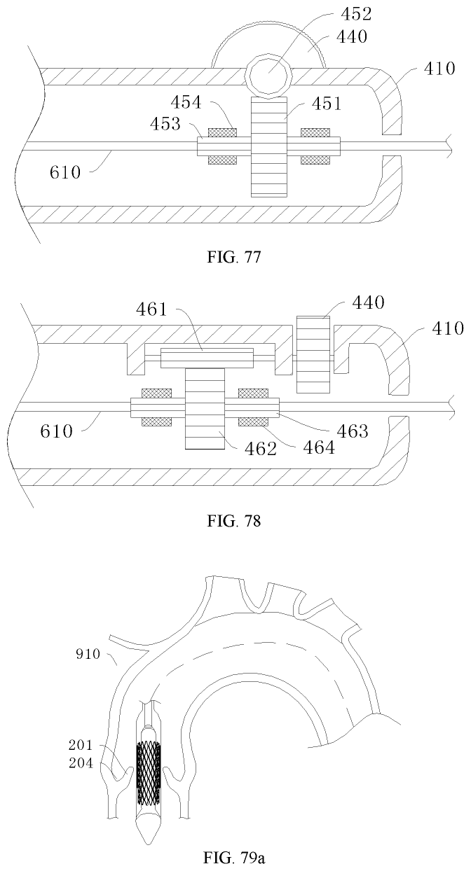

Referring to FIG. 77, in another embodiment, the rotatable seat 440 and the tube 610 are driven by a worm wheel 451 and a worm 452 engaging with each other. The rotatable seat 440 is configured as a wheel and rotatably mounted on the support 410, and the rotating axis of the rotatable seat 440 is perpendicular to the longitudinal direction of the support 410 (i.e., the extension direction of the tube 610). The rotatable seat 440 is coaxially fixed to the worm 452, and the worm wheel 451 is fixed to the tube 610 and engaged with the worm 452. A transmission sleeve 453 for reinforcing the tube 610 can be fixed to the outside of the tube 610, which is rotatably engaged with a support base 454 fixed on the support 410. The transmission sleeve 453 and the worm wheel 451 can be formed in one or separate pieces for transmission. When the rotatable seat 440 rotates, the torque is transmitted to the tube 610 through the worm gear mechanism for rotation.

Referring to FIG. 78, in another embodiment, the rotatable seat 440 and the tube 610 can be driven through a gear set. The gear set includes a first gear 461 and a second gear 462 that mesh with each other. For example, the rotatable seat 440 can be a wheel rotatably mounted on the support 410, and the rotating axis of the rotatable seat 440 is parallel to the extension direction of the tube 610. The rotatable seat 440 is coaxially fixed with the first gear 461 for transmission, and a transmission sleeve 463 is fixed to the outside of the tube 610 to reinforce its structure, and the transmission sleeve 463 is rotatably engaged with the support base 464 fixed on the support 410. The transmission sleeve 463 is coaxially fixed with the second gear 462 for transmission.

In the above embodiments that the tube 610 is driven by the rotatable base 440, a locking mechanism for limit the rotation of the rotatable seat 440 can be provided as required, for example, a pin slidably mounted on the support 410. The rotatable seat 440 is provided with an engagement slot or an insertion hole engaged with the pin to realize the position locking of the rotatable seat 440. In addition, a scale mark indicating a rotation angle can be provided on the rotatable base 440 to adjust the rotation angle of the tube 610.

An embodiment of the present application further provides an interventional system including the delivery system of the embodiments above, and the aortic prosthetic valve device 1000, wherein the aortic prosthetic valve device 1000 is disposed within the loading zone 406 of the delivery system.

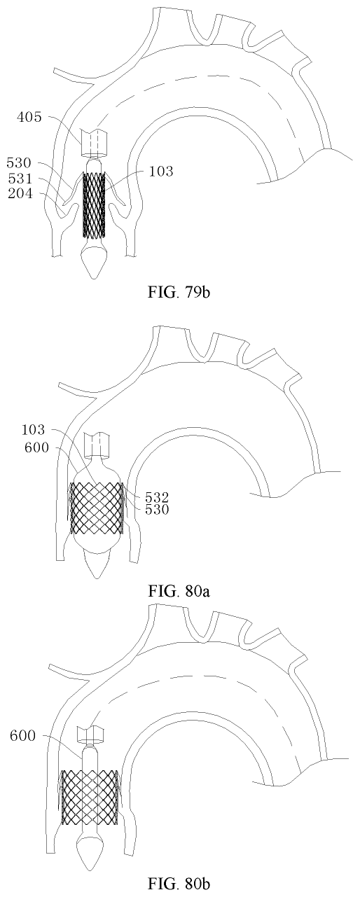

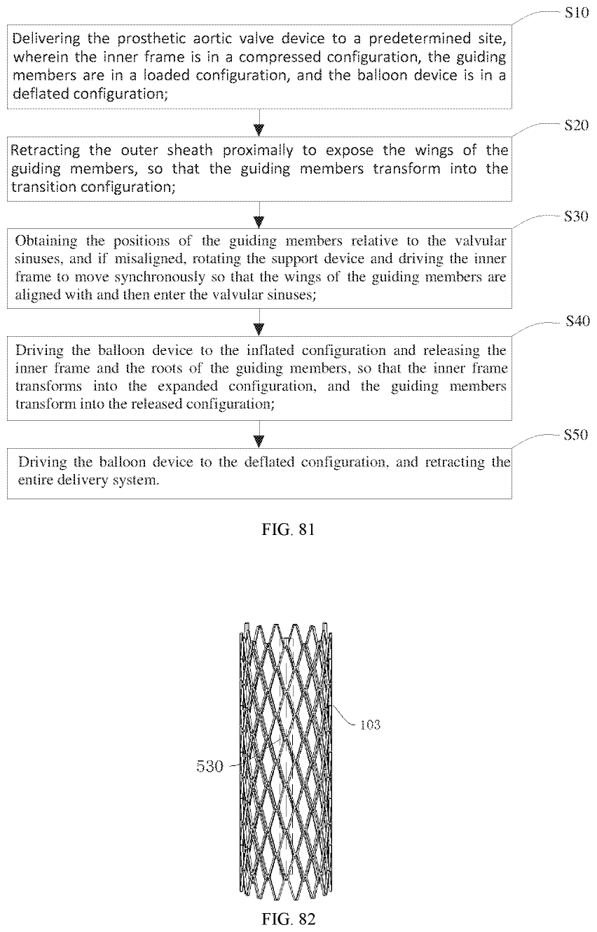

Referring to FIGS. 79a-81, an embodiment of the present application provides a method for using the above interventional system, which is also a method for securing the prosthetic heart valve device at an aortic annulus including a plurality of native valve leaflets, which can be implemented using the interventional system described above.

The method includes the following steps.

In step S10, as shown in FIG. 79a, the prosthetic aortic valve device 1000 is delivered to a predetermined site, wherein the inner frame 103 is in a compressed configuration, the guiding members 530 are in a loaded configuration, and the balloon device 600 is in a deflated configuration. In the delivery process, an imaging equipment can be used to detect and display the developing markers, and the spatial position of the prosthetic aortic valve device 1000 relative to the aortic valve can be determined by means of the contrast medium.

In step S20, as shown in FIG. 79b, after the prosthetic aortic valve device 1000 enters the native annulus (aortic annulus), the outer sheath 405 is retracted proximally to expose the wings 531 of the guiding members 530, so that the guiding members 530 made of the memory material tends to the preset configuration in the in vivo environment, with the wings expanding outward into the transition configuration, while the inner frame made of non-memory material is still in the compressed configuration, so that the roots of the guiding members do not obviously extend outward.

In step 530, the positions of the guiding members 530 in vivo, especially relative to the native annulus and valvular sinuses 204 can be obtained by using the imaging equipment in combination with the developing marker. Now, whether the circumferential positions of the guiding members 530 are aligned with the respective valvular sinuses can be initially determined. In some cases, for example, if the guiding members 530 are exactly aligned with the respective valvular sinuses, the prosthetic aortic valve device 1000 can be pushed further distally so that the free ends of the wings of the guiding members 530 generally abut the bottom of the valvular sinuses. If misaligned, the balloon device is rotated and the inner frame 103 is moved synchronously so that the wings 531 of the guiding members 530 are approximately aligned in the circumferential direction and then enter the valvular sinuses 204, and then the prosthetic aortic valve device 1000 is pushed distally, so that the free ends of the wings of the guiding members 530 further abut against the bottom of the valvular sinuses.

Since the guiding members are in the transition configuration, the wings thereof extend outward relative to the inner frame, so that at least one native valve leaflet enters the radial gap between the inner frame and the guiding member. At this time, it can be considered that the axial position of the prosthetic aortic valve device is desired. Preferably, all three native leaflets enter the respective radial gaps.

Otherwise, the whole device needs to be withdrawn proximally to readjust the position for ensuring clipping the native valve leaflets and the stability of the axial position after release.

In step S40, as shown in FIG. 80a, the balloon device 600 is driven to the inflated configuration by injecting fluid, that is, the inner frame 103 and the roots 532 of the guiding members 530 are released, so that the inner frame 103 transforms into the expanded configuration, and the guiding members 530 transform into the released configuration. Now, the prosthetic aortic valve device 1000 is released into position.

In the process of inflation and deformation of the balloon device, the two ends of the balloon in the axial direction suffer from relatively small radial restraint force, and thus will be first deformed, especially at the outflow end of the inner frame, which can drive the end of the inner frame together with the roots to turn over, so that the free ends of the wings tend to move closer to the inner frame to clip the native leaflets.

After the inner frame 103 transforms into the expanded configuration, the outflow section of the inner frame 103 can be substantially in a straight cylindrical configuration or turned over, depending on the pressure of the balloon or the shape of the balloon, and the roots radially move away from each other. Referring to the above deformation mechanism of the guiding members 530, the roots and the junctions with the wings deform so that the free ends of the wings of the guiding member 530 will further move closer to the outer wall of the inner frame 103 relative to the transition configuration to clip the native leaflets to ensure the positioning effect.

In step S50, as shown in FIG. 80b, after release, the balloon device 600 is switched to the deflated configuration, and the entire delivery system is retracted, while the prosthetic heart valve device is positioned and remained at the aortic annulus to replace the diseased native tissue.

In the present application, the prosthetic aortic valve device 1000 is improved in structure to facilitate circumferential position adjustment, aligning the valve leaflets 200 with the coronary orifice to reduce blood flow interference, and further avoiding positional deviation during long-term use.

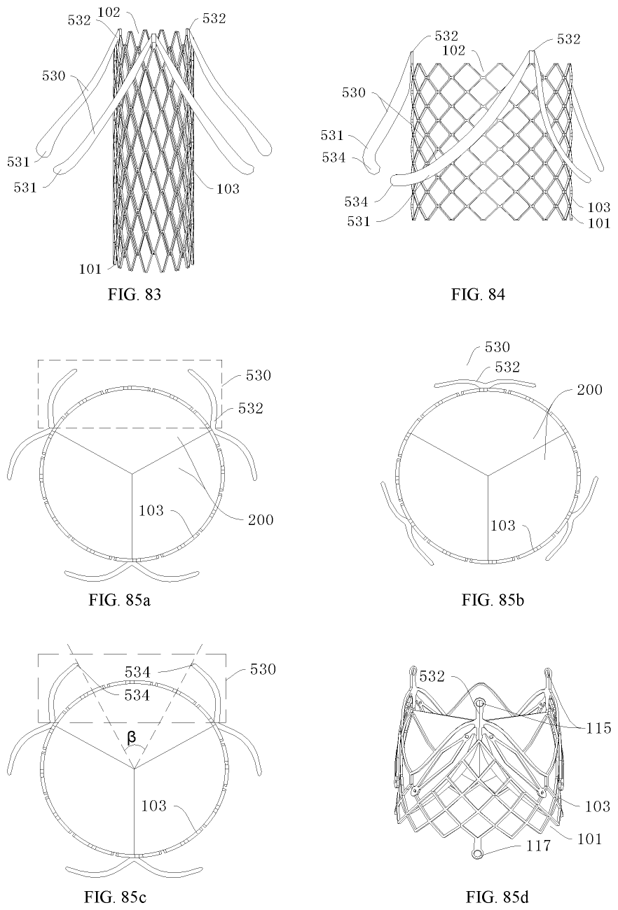

Referring to FIGS. 82-84, in a prosthetic aortic valve device according to another embodiment, two separate wings 531 are provided, that is, the individual guiding member 530 has two wings.

In the loaded configuration, the guiding members 530 (shown in dashed lines) are radially pressed against the inner frame 103 in the compressed configuration, so that the inner frame 103 and the guiding members can be easily surrounded by the sheath and delivered in vivo.

In the transition configuration, the roots 532 of the guiding members 530 remain gathered to adapt the compressed configuration of the inner frame 103. The wings 531 are self-deformed and thus extend outside of the inner frame 103, with an accommodation space formed between the outer wall of the inner frame 103 and the wings 531 for receiving the native leaflets 201. In order to circumferentially position the inner frame 103, the extended wings 531 can be adjusted in position to enter the corresponding valvular sinuses, with the inner frame 103 inside the native leaflets and the wings 531 outside the native leaflets.

In the released configuration, the roots 532 of the guiding members 530 move away to adapt the expanded configuration of the inner frame 103, at which time both the inner frame 103 and the guiding members 530 are fully released from the delivery system into the work state.

FIGS. 82-84 are only for illustration of the spatial posture and relative relationship or characteristics in different configurations. Unless otherwise specified, the shape and position of the guiding member 530 are described referring to the released configuration, and the shape and position of the inner frame 103 are described referring to its expanded configuration.

The meshed cylindrical structure can be radially deformed to facilitate the intervention after compression and the subsequent expansion and release. The axial length of the meshed cylindrical structure may change when the meshed cylindrical structure is radially deformed. The meshed cylindrical structure is configured to be expanded by external force, i.e., the meshed cylindrical structure is not made of self-expandable material. In general, the meshed cylindrical structure can be expanded by a balloon. However, the guiding member 530 is made of a memory material (e.g., pre-heat-set nickel-titanium alloy), which can be released in the human body first, the root 532 of which can be considered as a portion where the guiding member 530 and the inner frame 103 are adjacent and connected to each other. The specific shape is not strictly limited. The root 532 and the wing 531 can be formed in one piece to facilitate processing. The wing 531 extends outward relative to the inner frame 103 after release, and by adjusting the posture of the inner frame 103, the wing 531 can enter into the valvular sinus 204 to pre-position the inner frame 103 in the circumferential direction, and then the inner frame 103 can be released and expanded by a balloon. Because the guiding members 530 are aligned with the valve leaflets 200, the junction of adjacent valve leaflets 200 avoids the coronary artery orifice and prevents the blood flow from being obstructed. In addition, the wings 531 abut against the bottom of the valvular sinuses 204, which positions the inner frame 103 in the axial direction to avoid slipping to the left ventricle side under the action of the reverse flow of blood.

In order to fix the guiding member 530, as shown in FIGS. 85a and 85d, the junction of two adjacent leaflets 200 on the inner frame 103 is the commissure region of the inner frame 103, and the root 532 of the guiding member 530 is fixed to a corresponding junction. Alternatively, the root 532 of the guiding member 530 can be located between two adjacent junctions in the circumferential direction of the inner frame 103.

Referring to FIG. 85d, in the released configuration, the free ends 534 of the two wings 531 of the individual guiding member 530 are spaced apart from each other, and the spacing region has a central angle P, in the circumferential direction of the inner frame 103, greater than 30 degrees.

The commissure region can be a strip-shaped, i.e., commissure post 132, and each commissure post 132 can be provided as follows.

The commissure post 132 can extend from the end node of the outflow end 102 of the inner frame 103 or be located within the inner frame 103. The commissure post 132 extends along the axis of the inner frame 103 or is inclined radially inward. For example, the outflow end 102 of the inner frame 103 can have a structure with peaks and valleys, and the commissure region is located at the peak, that is, at the most-distal end of the outflow end of the inner frame 103.

Referring to FIG. 85d, the end of the commissure post 132 is provided with a first collar 115, and the inner frame 103 is provided at the inflow end 101 with a second collar 117 in alignment with the first collar 115. The first collar 115 and the second collar 117 can be used for providing developing marker or can be used to connect with the delivery system as required.

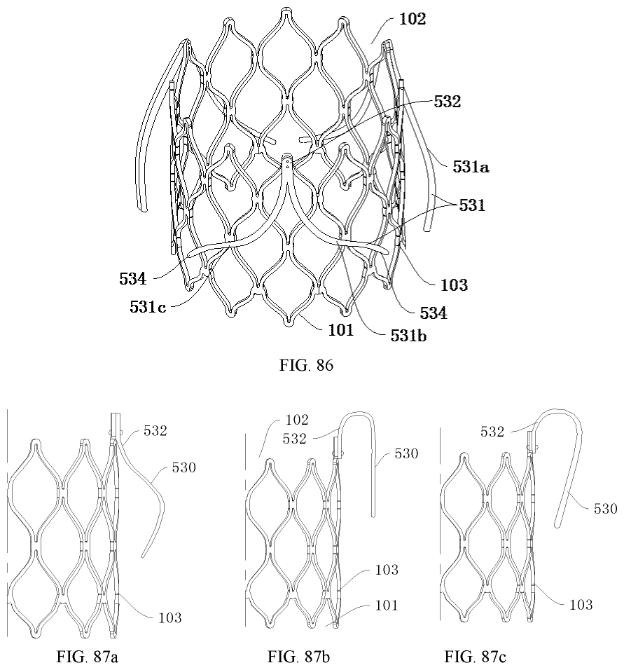

Referring to FIG. 86, each guiding member 530 can include two wings 531a and 531b, the ends of which facing away from the inner frame 103 are separate free ends 534. In one guiding member, failure of one of the free ends 534 to enter the valvular sinus does not necessarily affect the other free end, thus avoiding to some extent the risk of failure of the guiding member as a whole.

In the axial direction of the inner frame 103, the free end 534 is located adjacent to the inflow end 101 of the inner frame 103, and the root 532 is located adjacent to the outflow end 102 of the inner frame 103, so that the wing 531 has a sufficient extension to ensure positioning. In order to improve safety, the free end 536 has a rounded structure, and can be further covered with a protective layer.

Taking FIG. 86 as an example, in two adjacent guiding members, the wing 531b and the wing 531c, which are close to each other, are formed in one piece by a common root 532, and the common root 532, the wing 531b and the wing 531c form a branched structure, the opening of which faces towards the inflow end 101. This branched structure facilitates crossing the junction of the two native leaflets by virtue of its opening such that the guiding members can be respectively positioned in the respective sinuses.

FIGS. 87a-c show three alternative configurations of the guiding member 530 in the released configuration, including: a first example, in which the guiding member 530 extends outward from the root 532 in the radial direction of the inner frame 103, and then is bent inward, as shown in FIG. 87a; a second example, in which the guiding member 530 extends from the root 532 in the axial direction of the inner frame 103 toward the outflow end 102 and is bent toward the inflow end 101, as shown in FIG. 87b; and a third example incorporating the first and the second examples, as shown in FIG. 87c.

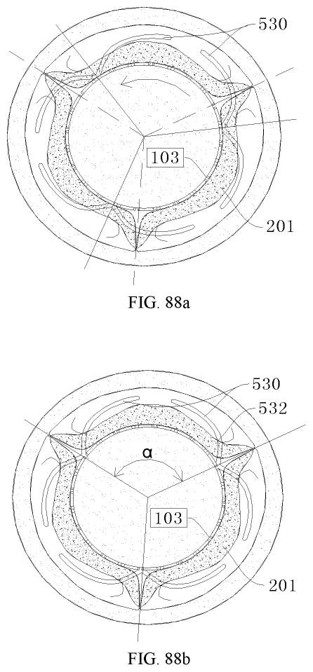

Referring to FIGS. 88a and 88b, in order to exactly guide the inner frame 103 to position and reduce the offset once in place, the guiding member 530 needs to have a sufficient circumferential span, which can be a span of different portions, for example, a portion of the root 532 or the wing 531, wherein the root 532 having the largest circumferential span is more advantageous for stabilizing the position of the inner frame 103. For example, take the root 532 as an example: in the circumferential direction of the inner frame 103, each guiding member 530 spans at least ⅙ circumference, i.e., the center angle a in FIG. 92b is greater than or equal to 60 degrees. Further, for example, each guiding member 530 spans ⅓ circumference in the circumferential direction of the inner frame 103, that is, the central angle a is equal to 120 degrees.

In order to facilitate smooth entry of the guiding member into the valvular sinus in the case where the root 532 has a large span, the guiding member has opposite outer and inner sides in the circumferential direction of the inner frame, and the edge of the wing on the outer side of the guiding member has a smooth contour. In addition, the curve of the contour extends from the root to the inflow end and is offset toward the inner side. The smooth contour and the extension of the curve facilitate the positioning of the guiding member itself in the valvular sinus, reducing the difficulty of adjusting and positioning the inner frame, and additionally reducing the potential safety hazard and avoiding puncturing the surrounding tissue.

After release of the guiding members 530, there may be deviations in the circumferential positions of the guiding members 530 from the positions of the valvular sinuses 204. For example, the areas represented by the three radially extending solid lines can be regarded as the approximate distribution areas of the three guiding members, while the areas represented by the three radially extending dotted lines can be regarded as the approximate distribution areas of the three valvular sinuses, which are not aligned with each other as shown in the figure, in which case, the inner frame 103 can be rotated in the direction of the solid arrow shown in the figure to drive the guiding members 530 until the three radially extending solid lines coincide with the dashed lines, so as to achieve circumferential alignment as shown in FIG. 92b.

After circumferential alignment, the inner frame 103 is moved toward the inflow end until the guiding members 530 abut against the bottom of the valvular sinuses 204 to achieve positioning. In the radial position, the native leaflet 201 is located between the guiding member 530 and the inner frame 103. The inner frame 103 can be then released and expanded by a balloon, thereby avoiding the coronary artery.

In the released configuration, the ratio of the axial length of the guiding members 530 to the entire length of the inner frame 103 is 40% to 80%, for example, 50%.

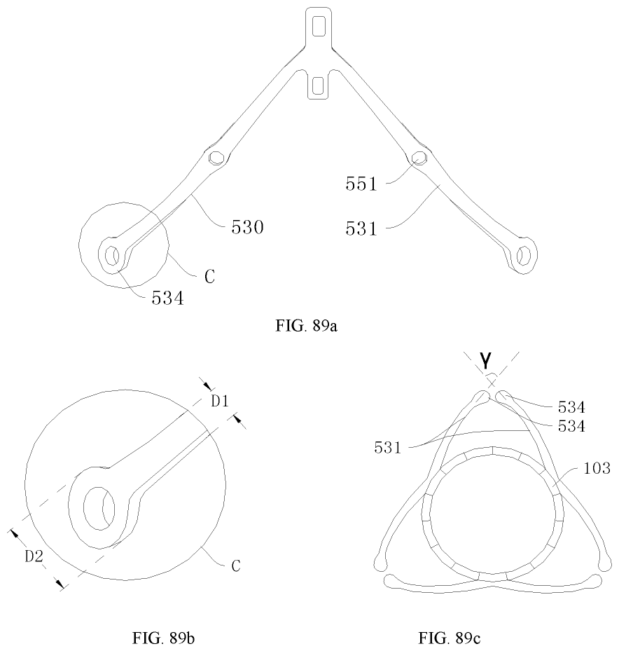

Referring to FIGS. 89a and 89b, in one embodiment, the free end 534 of the guiding member is configured as a ring structure with a smoothed outer periphery, the wing 531 is generally strip-shaped and has opposite length and width directions, and the width of the ring structure is larger than that of the wing 531. The width D2 of the ring structure is 2 to 6 times the width DI of the wing 531.

Referring to FIG. 89c, the free end 534 defines a reference plane, and in the transition configuration, the free ends 534 of the two wings 531 of the individual guiding member define a first reference plane and a second reference plane, respectively, and the angle y between the first reference plane and the second reference plane is less than or equal to 90 degrees, preferably less than 45 degrees, for example 45 degrees.

Referring to FIG. 89d, each guiding member 530 includes two wings 531. In two adjacent guiding members 530, a first wing 5311 of one of the guiding members 530 and a second wing 5312 of the other guiding member 530 are adjacent to each other in the circumferential direction of the inner frame 103. The outflow end 102 of the inner frame is provided with commissure posts 132, and the roots of the first wing 5311 and the second wing 5312 are connected to each other to form one piece which is overlapped and fixed to the outer side of the respective commissure post 132.

In connection with the above embodiments, the first wing 5311 and the second we 5312 are connected to a common root 532, which three can be considered to constitute one group of clipping arms. The prosthetic aortic valve device as a whole has three groups of clipping arms, and each group of clipping arms is separately connected to the inner frame.

In the released configuration, the first wing 5311 and the second wing 5312 are almost coplanar.

The roots 532 corresponding to the two wings 531 of the individual guiding member 530 are formed in one or separate pieces. In the case where the free ends 534 are separate from each other, if one of the wings were worked out, it would not pull the other, and since the roots 532 are close to the inner frame 103, the two wings 531 would not be pulled by each other. Taking the separate roots 532 as an example, the span of the guiding member 530 in the circumferential direction of the inner frame 103 can be understood as the central angle between the lines connecting the two roots 532 and the center of the inner frame 103, i.e., the central angle a shown in FIG. 92b.

Each wing 531 has a flat strip structure as a whole. The flat strip structure can be solid or totally hollowed out (leaving only the edge bars) or partially hollowed out (e.g. a meshed structure), for example by weaving or cutting. The flat strip structure can have a certain width, but does not necessarily extend with an equal width. The “flat” shape is more favorable for reducing the overall radial dimension during loading and ensuring compliance during intervention, while the “strip” shape is more favorable for space shaping.

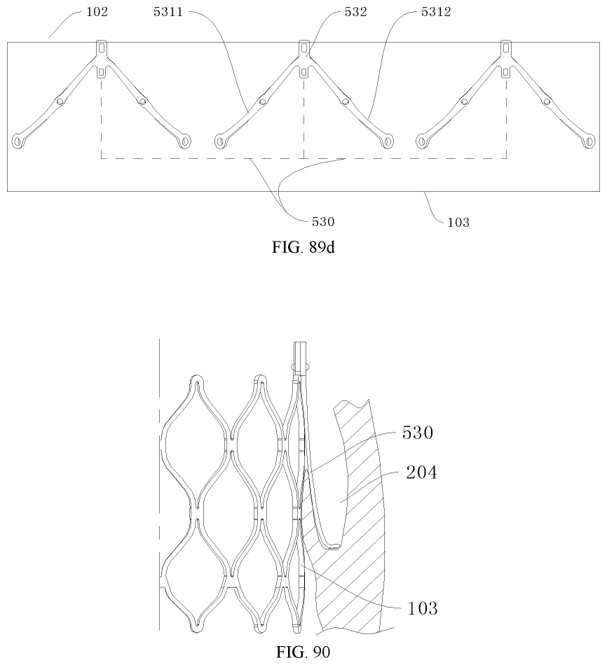

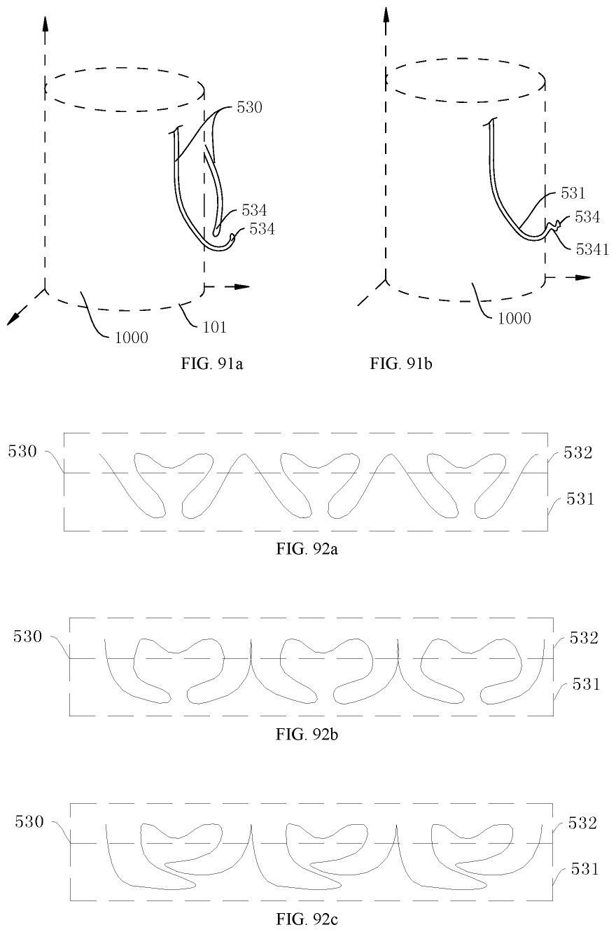

Referring to FIG. 90 to FIG. 91b, the two wings 531 extend toward the inflow end 101 respectively from two outer sides of the respective guiding member 530, and approach each other.

The wing 531 generally has an arc configuration, and a wave structure 5341 can be provided adjacent to the free end 534 thereof, which can undulate in the radial and/or axial direction of the inner frame 103, or extend in the circumferential direction of the inner frame 103 at a section adjacent the free end 534. In the figure, the wave structure 5341 mainly undulates in the axial direction of the prosthetic aortic valve device. It will be conceived that the guiding member 530 can have undulations in multiple dimensions in three dimensions. Referring to the drawings, the guiding member 530 has a radially undulating structure in an axial view of the inner frame 103. The undulations in multiple directions can be provided separately or overlapped with each other to form a complex three-dimensional configuration.

In the transition configuration, the two wings 531 of the individual guiding member 530 have expanded outward, but the roots 532 of the two wings are restrained by the configuration of the inner frame 103 and are still adjacent to each other, in which case, the free ends 534 of the two wings 531 should not interfere with each other. Therefore, in the circumferential direction of the inner frame 103, the free ends 534 of the two wings 531 of the individual guiding member 530 are staggered with each other in the transition configuration of the guiding member 530, and in the released configuration, the free ends 534 of the two wings 531 in the individual guiding member 530 are spaced from each other.

The free ends 534 of the two wings 531 in the individual guiding member 530 are staggered with each other in the transition configuration of the guiding member 530 in such a way that the free ends 534 of the two wings 531 in the individual guiding member 530 are spaced in the radial or axial direction of the inner frame 103.

For ease of processing, referring to FIGS. 92a-92c, all of the guiding members 530 are formed in one piece, for example, by bending a strip of metal. Each of the wings 531 extends from opposite outer sides of the guiding member 530, which ensures the overall circumferential span of the guiding member 530. In the figure, the guiding members 530 have different shapes. In FIG. 92b, the guiding members 530 extend approximately in the axial direction of the inner frame 103, and then turn to extend approximately in the circumferential direction of the inner frame 103. As shown in FIG. 92c, the guiding members 530 in the same group can be provided asymmetrically.

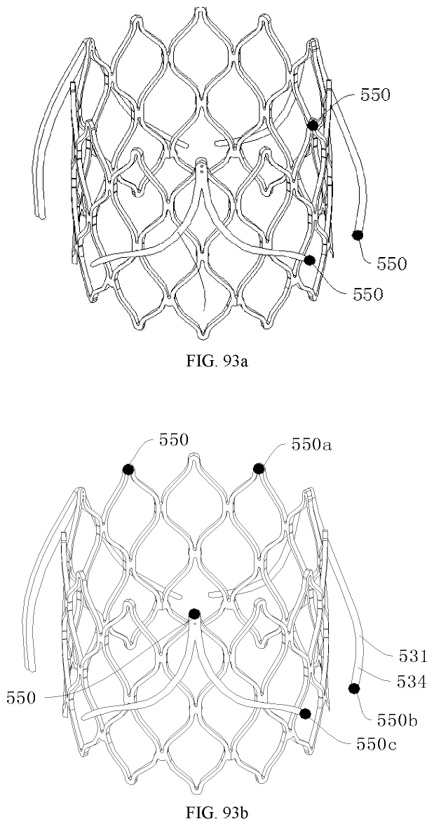

Referring to FIGS. 93a-93b, in cooperation with an imaging equipment, the prosthetic aortic valve device 1000 can be provide with a developing marker 550, which can be embedded or include a precious metal that can be displayed differentiating from other portions under X-ray or other means of detection.

The developing marker 550 can be in the form of a dot or a strip or a ring (closed or non-closed, but at least in half ring), and the developing marker 550 can be disposed in at least one of the inner frame 103 and the guiding members 530. For example, the inner frame 103 or the guiding members 530 are provided with eyelets for receiving the developing marker 550.

The developing marker 550 is installed, but not limited to one or more of the following methods: the wings 531 of the at least two guiding members 530 are provided with the developing markers 550, and preferably, the wings 531 of all the guiding members 530 are provided with the developing markers; the roots 532 of at least two of the guiding members 530 are provided with the developing markers 550, and preferably the roots 532 of all the guiding members 530 are provided with the developing markers.

In the axial view of the inner frame 103, at least three developing markers 550 are visible and are distributed in different regions in the circumference of the inner frame 103. The position of the axis of the inner frame 103 can be determined according to the shape formed by the developing markers 550 (displayed in the imaging equipment), so as to determine whether there is excessive tilt or the like.

In that axial view of the inner frame 103, at least three developing markers 550 are visible and at least two are distributed in different regions in the radial direction of the inner frame 103. The developing markers 550 in different regions in the radial direction can assist in determining the posture of the inner frame 103 in the circumferential direction.

In a radial view of that inner frame 103, at least three developing markers 550 are visible and are distributed in different regions in the axial direction of the inner frame 103, in order to determine the position of the axis of the inner frame 103 from the radial view.

At least one developing marker is disposed in the inner frame 103 or the root 532 of the guiding member 530, and at least one developing marker is disposed in the wing 531 of the guiding member 530 and adjacent to the free end 534 of the wing 531, in order to determine the extension of the respective wing 531.

In combination of the above methods, as shown in FIG. 93b, for example, a first developing marker 550a is arranged in the inner frame 103, a second developing marker 550b and a third developing marker 550c are arranged in the free ends 534 of the two wings 531. All these developing markers 550 are distributed in different regions in the circumferential direction of the inner frame 103 from the axial view of the inner frame 103, and the first developing markers 550a and the other two developing markers are distributed in different regions in the radial direction of the inner frame 103, and the first developing marker 550a and the other two developing markers are also distributed in different regions in the axial direction of the inner frame 103. By means of the contrast medium, the posture of the prosthetic aortic valve device 1000 in the aorta and the alignment of the guiding members 530 with the valvular sinuses 204 can be easily determined.

The technical features of the above embodiments can be arbitrarily combined, and not all possible combinations of the technical features of the above embodiments have been described for the sake of brevity of description. However, as long as there is no contradiction in the combination of these technical features, it should be regarded as falling in the scope of this specification. When the technical features in different embodiments are shown in the same figure, it can be considered that the drawing also discloses a combination example of various embodiments involved.

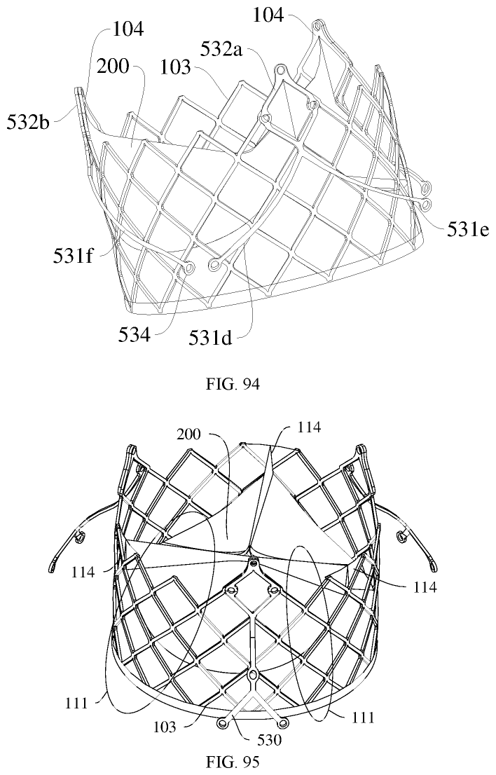

For example, FIG. 94 can be considered as showing another embodiment which combines FIGS. 70a to 70c which shows the shape characteristics and the spatial deformations of the joint between the root and the wing with FIGS. 86, 89a, 19, or 37a or the like which shows the specific configurations of the wing.

Connecting posts 104 extend from the outflow end of the inner frame 103. The connecting post 104 is V-shaped and the sharp corner of the V-shape is axially convex, and the junction of the two adjacent leaflets 200 on the inner frame 103 is the commissure region of the inner frame 103. The connecting posts 104 are located at the respective commissure region in the circumferential direction, which is different from what shown in FIG. 46a, where the connecting post is located between adjacent two commissure regions.

In the following, the positioning structure for the prosthetic heart valve (which can also be regarded as the prosthetic aortic valve when applied to the aorta) in this embodiment will be described from different perspectives.

For the guiding member, there are three circumferentially arranged guiding members respectively corresponding to three valvular sinuses in the human body, and the guiding member includes two separate roots, for example a root 532a and a root 532b. The roots 532a and 532b are respectively connected to different connecting posts 104, the roots 532a further extends to form a wing 53 Id, the roots 532b further extends to form a wing 53 If, and the free ends 534 of the wings 53 Id and 53 If are separate of each other.

For the clipping arm, there are three groups of clipping arms arranged in circumferential direction, each group including two clipping arms. For example, one of the clipping arms has a root 532a which further extends to form two branched wings, wing 53 Id and wing 53 le, respectively, wherein the free ends 534 of the wing 53 Id and the wing 53 If in the other group of clipping arms are adjacent to each other and correspond to the same valvular sinus in vivo, which is more advantageous for avoiding coronary arteries.

The above different perspectives refer to the same structure. In this embodiment, the connection of the root and the wing refers to FIGS. 70a to 70c (the reference numerals in which are applied in the following). From the clipping arm, the root is fixed to the outer side of the inner frame by binding, including a first bar and a second bar, the wing includes, adjacent to the root, a third bar and a fourth bar, wherein one end of the third bar is connected to the first bar, and the other end of the third bar extends toward the inflow end; one end of the fourth bar is connected to the second bar, and the other end of the fourth bar extends toward the inflow end and intersects with the third bar. The third and fourth bars meet and then diverge away from each other until they extend to the free ends, with the different branches (such as the wing 53 Id and the wing 53 le respectively in FIG. 94) corresponding to different valvular sinuses. The first bar 5321, the second bar 5322, the third bar 5351, and the fourth bar 5352 form a quadrangle, and the principle and deformation of the quadrangle refer to the above, and would not be repeated herein.

In accordance with the above embodiments, the clipping arm or the guiding member at the periphery of the inner frame can be regarded as a positioning member, which play in important role in circumferential alignment with the valvular sinus and in shifting restriction of the frame in the axial direction. The positioning member being not directly connected with the two commissure regions in the circumferential direction also ensures the positioning effect.

In summary, the present application provides a prosthetic aortic valve device having opposite inflow and outflow ends, the prosthetic aortic valve device including:
an inner frame 103 having a meshed cylindrical structure, which is radially deformable and has relative compressed configuration and expanded configuration after being subjected to an external force, wherein the interior of the inner frame 103 is configured as an axially through blood flow channel 301;
valve leaflets 200 connected to the inner frame 103 and cooperating with each other to control the blood flow channel, with the junctions of the two adjacent valve leaflets 200 on the inner frame 103 being the commissure region 114; and
positioning members (the above-mentioned clipping arm 120 or the guiding member 530) arranged in sequence in the circumferential direction of the inner frame 103, one end of each of which is connected to the inner frame 103 and the other end extends toward the inflow end, wherein a spacing region 111 is formed at the outer peripheral region of the inner frame between two adjacent commissure regions 114, and the positioning members avoid the spacing region 111.

Due to the spacing region 111, any positioning member would not directly connect the two commissure regions 114. For example, in FIG. 95, the positioning member can be considered as a guiding member 530 located between adjacent two commissure regions 114, without being connected to the commissure regions 114 in the circumferential direction. Therefore, there are two spacing regions 111 between two adjacent commissure regions 114 in addition to the guiding member 530. The circumferential span of the guiding member 530 is limited, in order to realize circumferential alignment with valvular sinus.

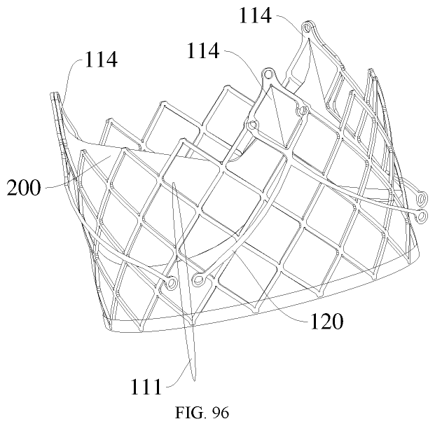

As another example, in FIG. 96, the free ends of the wings (from different clipping arms 120) on either side of the spacing region 111 are separate of each other and are not directly connected, providing more anchor points with the valvular sinus, reducing the risk of anchor failure.

In connection with the foregoing, the positioning member is made of a memory material and is configured to be switchable in the following configuration:
in the loaded configuration, the positioning members are radially pressed to contact the inner frame 103 in the compressed configuration;
in the transition configuration, the ends of the positioning members connected to the inner frame 103 are gathered together to adapt the inner frame 103 in the compressed configuration, the ends of the positioning members extending toward the inflow end self-extend at the outer peripheral region of the inner frame 103, with an accommodation space formed between the positioning members and the outer wall of the inner frame for receiving the native leaflets; and
in a released configuration, the ends of the positioning members connected to the inner frame 103 move away from each other to adapt the expanded configuration of the inner frame 103.

The positioning member connected to the individual commissure region 114 is not directly connected to the other commissure regions, i.e., the positioning members are a plurality of separately configured members. The positioning member is at most directly connected to one commissure region 114 (in the case where the guiding member 530 is connected between the two commissure regions 114, it can be considered not to be directly connected to either commissure region).

The inner frame 103 is released in a balloon expansion manner to allow the transition configuration of the positioning members. For other structural details of the prosthetic aortic valve device and the method for use in vivo, reference is made to the foregoing embodiments.

The above-described embodiments only represent several embodiments of the present application, and the description therefor is specific and detailed, but should not be construed as limiting the scope of the patent application. It should be noted that a number of modifications and developments can be made to those of ordinary skill in the art without departing from the spirit of the present application, all of which are within the scope of protection of the present application.

1-22. (canceled)

23. A frame for a prosthetic heart valve device, comprising:
an inner frame having a meshed cylindrical structure, the inner frame, depending on a radial deformation, having relative compressed and expanded configurations, and an interior of the inner frame being capable of receiving a support device for driving the inner frame to transform into the expanded configuration; and
a plurality of groups of clipping arms located around an outer periphery of the inner frame and spaced apart from each other in a circumferential direction of the frame, each clipping arm having opposite fixed and free ends, the fixed end being directly or indirectly connected with the inner frame, and the fixed ends of the clipping arms in the same group are adjacent to each other, and wherein the clipping arm is made of a memory material and has configurations of:
a loaded configuration, in which the inner frame assumes the compressed configuration, and the clipping arms contact the inner frame; and
a released configuration, in which the inner frame assumes the expanded configuration, and the free ends of the clipping arms expand radially outward, with a space defined between the free ends of the clipping arms and the inner frame for allowing entry of native leaflets, and wherein the free ends of at least two clipping arms in the same group tend to extend away from each other, and the free ends of at least two clipping arms in adjacent groups tend to extend close to each other.

24. The frame for a prosthetic heart valve device of claim 23, wherein the inner frame is provided with at least two commissure regions spaced apart from each other in the circumferential direction, and the fixed ends of the clipping arms in the same group are connected with a respective commissure region; and in the loaded configuration, the inner frame and all the clipping arms do not radially overlap with each other.

25. The frame for a prosthetic heart valve device of claim 24, wherein the commissure region comprises a commissure post, a frame arm is connected between adjacent commissure posts, with an angled space defined between the frame arm and an outflow end of the inner frame, and the clipping arms are located within the respective angled spaces in the loaded configuration; and
the frame arm is configured as a single rod or a deformable mesh band.

26. The frame for a prosthetic heart valve device of claim 23, wherein the fixed end is configured as a root and is fixed with an outer side of the inner frame by banding, and the clipping arm has a wing extending from the fixed end toward the free end;
the root includes a first bar and a second bar, and the wing includes a third bar and a fourth bar adjacent to the root, wherein the first bar, the second bar, the third bar, and the fourth bar form a quadrilateral; and
ends of the third and the fourth bars away from the root meet and then extend away from each other in a branched manner until the respective free ends, wherein the branches are configured to correspond to different valvular sinuses.

27. The frame for a prosthetic heart valve device of claim 23, wherein a single or a plurality of clipping arms are connected with the same side of the commissure region in the circumferential direction of the inner frame, and wherein,
the free end of the single clipping arm has a branched structure, or a middle portion of the single clipping arm has a branched structure with a one-piece free end; and
the plurality of clipping arms are separate from each other or the free ends thereof are formed in one piece.

28. The frame for a prosthetic heart valve device of claim 23, wherein the clipping arm is configured as a single rod or a deformable mesh band, and the mesh band is deformable in a direction in which the clipping arm extends.

29. The frame for a prosthetic heart valve device of claim 23, wherein the clipping arm extends from the fixed end toward an inflow end of the inner frame; and the free ends of at least two clipping arms in the same group tend to extend away from each other, and the free ends of at least two clipping arms in two adjacent groups tend to extend close to each other.

30. The frame for a prosthetic heart valve device of claim 23, wherein the clipping arm has a wavy structure adjacent the free end thereof.

31. The frame for a prosthetic heart valve device of claim 23, wherein the fixed ends of the clipping arms in the same group converge to a connecting portion and are fixed with the inner frame through the connecting portion;
the connecting portions corresponding to the clipping arms in the same group are formed in one piece or separate pieces adjacent each other; and
the clipping arms in the same group include multiple pairs of clipping arms, wherein the clipping arms in the same pair are respectively located on two sides of the connecting portion in the circumferential direction of the inner frame, and the released clipping arms in different pairs have different lengths.

32. The frame for a prosthetic heart valve device of claim 23, wherein the fixed ends of the clipping arms in the same group converge to a connecting portion and are fixed with the inner frame by the connecting portion; and
the clipping arms in the same group include multiple pairs of clipping arms, wherein in the circumferential direction of the inner frame, the clipping arms in the same pair are respectively located on two sides of the connecting portion, and the clipping arms in different pairs on the same side of the connecting portion tend to extend differently in the released configuration.

33. The frame for a prosthetic heart valve device of claim 23, wherein, in the released configuration, the free ends of the clipping arms in the same group are in the same position or offset from each other in a radial direction of the inner frame; and
in the released configuration, the free ends of all the clipping arms are located between two ends of the inner frame in an axial direction of the inner frame and adjacent to an inflow end of the inner frame.

34. The frame for a prosthetic heart valve device of claim 23, wherein the fixed ends of the clipping arms in the same group converge to a connecting portion and are fixed to the inner frame through the connecting portion; and the inner frame is provided with at least two commissure regions spaced from each other in the circumferential direction, and the connecting portion is fixed with the respective commissure region of the inner frame by welding or by connecting members; and
in a radial direction of the inner frame, the connecting portion is overlapped on an outer side of the commissure region; or in the circumferential direction of the frame, the connecting portion is located at a circumferential side of the commissure region.

35. A frame for a prosthetic heart valve device, comprising:
an inner frame having a meshed cylindrical structure, the inner frame, depending on a radial deformation, having relative compressed and expanded configurations, and an interior of the inner frame being capable of receiving a support device for driving the inner frame to transform into the expanded configuration;
a connecting ring fixed at an outflow end of the inner frame and provided with a plurality of connecting regions spaced apart from each other; and
a plurality of groups of clipping arms located around an outer periphery of the inner frame and spaced apart from each other in a circumferential direction of the frame, each clipping arm having opposite fixed and free ends, and the fixed ends of clipping arms in the same group are located at the same connecting region; wherein
the clipping arm is made of memory material and has configurations of
a loaded configuration, in which the inner frame assumes the compressed configuration, and the clipping arms contact the inner frame; and
a released configuration, in which the inner frame assumes the expanded configuration, and the free ends of the clipping arms expand radially outward, with a space defined between the free ends of the clipping arms and the inner frame for allowing entry of native leaflets.

36. The frame for a prosthetic heart valve device of claim 35, wherein the connecting ring and the clipping arms are formed by winding a wire.

37. A frame for a prosthetic heart valve device, comprising:
an inner frame having a meshed cylindrical structure, the inner frame, depending on a radial deformation, having relative compressed and expanded configurations, and an interior of the inner frame being capable of receiving a support device for driving the inner frame to transform into the expanded configuration; and
clipping arms, each clipping arm having opposite fixed and free ends, the fixed end being connected with the inner frame and extending in a circumferential direction of the frame, and the clipping arm satisfying at least one of the following conditions with respect to an axis of the inner frame:
a circumferential distribution region M1 of the fixed end with respect to the axis has a central angle greater than 15 degrees; and
an axial distribution region M3 of the clipping arm with respect to the axis has a length greater than 5 mm; wherein
the clipping arm is made of memory material and has configurations of:
a loaded configuration, in which the inner frame assumes the compressed configuration, and the clipping arms contact the inner frame; and
a released configuration, in which the inner frame assumes the expanded configuration, and the free ends of the clipping arms expand radially outward, with a space defined between the free ends of the clipping arms and the inner frame for allowing entry of native leaflets.

38. The frame for a prosthetic heart valve device of claim 37, wherein the clipping arms are provided in groups and the fixed ends of the clipping arms in the same group are adjacent to each other, and a circumferential distribution region M4 of the fixed ends of each group of clipping arms with respect to the axis has a central angle of 360/n or less, where n is the number of the leaflets configured to be loaded in the frame.

39. The frame for a prosthetic heart valve device of claim 37, wherein the circumferential distribution region M1 of the fixed end of the individual clipping arm with respect to the axis has a central angle of less than or equal to 360/2n, where n is the number of the leaflets configured to be loaded in the frame.

40. The frame for a prosthetic heart valve device of claim 37, wherein each clipping arm comprises an enlarged positioning structure located at the free end thereof and formed by extension of the clipping arm's own material; or the positioning structure is located at a side edge of the clipping arm extending from the fixed end to the free end thereof.

41. The frame for a prosthetic heart valve device of claim 37, wherein each clipping arm is covered with a sleeve being a woven structure or formed in one piece.

42. The frame for a prosthetic heart valve device of claim 37, wherein, in the loaded configuration, the clipping arms in the same group are close to each other and around an outer periphery of the inner frame; and
the clipping arms do not overlap each other in a radial direction of the inner frame.

43-76. (canceled)
